(19) United States
(12) Patent Application Publication
(10) Pub. No.: US 2026/0007763 A1
(43) Pub. Date: Jan. 8, 2026

(54) PEGYLATED ANTIBODY HYDROXYL-BEARING DRUG CONJUGATE

(71) Applicant: Shenzhen Enduring Biotech, Ltd., Shenzhen, Guangdong (CN)

(72) Inventors: Dechun Wu, Shenzhen, Guangdong (CN); Shumin Liu, Shenzhen, Guangdong (CN); Shuqiang Yin, Shenzhen, Guangdong (CN); Yu Wen, Shenzhen, Guangdong (CN)

(73) Assignee: Shenzhen Enduring Biotech, Ltd., Shenzhen, Guangdong (CN)

(21) Appl. No.: 18/836,485

(22) PCT Filed: Feb. 13, 2023
(86) PCT No.: PCT/CN2023/075676

§ 371 Date: Aug. 7, 2024

(30) Foreign Application Priority Data
Feb. 11, 2022 (WO) PCT/CN2022/076012

(51) Int. Cl. A61K 47/68; A61P 35/00

(52) U.S. Cl. CPC A61K 47/68037; A61K 47/6803; A61K 47/6879; A61P 35/00

(57) ABSTRACT

Provided herein is an antibody-drug conjugate (ADC) especially a PEGylated mono- or bispecific antibody hydroxyl-bearing drug conjugate prepared with site-specific conjugation to provide homogeneous conjugate with high potency and low toxicity. The disclosure also relates to a method for the preparation of the antibody hydroxyl-bearing drug conjugate, a composition comprising the antibody hydroxyl-bearing drug conjugate, and the use thereof in treating diseases.

### CROSS REFERENCE TO RELATED APPLICATIONS

This application is a National Stage application under 35 U.S.C. 371 of International PCT Application No. PCT/CN2023/075676, filed on Feb. 13, 2023, which claims priority to International PCT Application No. PCT/CN2022/076012, filed Feb. 11, 2022, the entire contents of which are incorporated herein by reference.

### FIELD OF INVENTION

The present invention relates to an antibody hydroxyl-bearing drug conjugate, especially a multi-specific antibody hydroxyl-bearing drug conjugate for which the hydroxyl group of the payload is used to link a drug to an antibody. In particular, this invention relates to a long acting PEGylated mono- or bispecific single chain antibody drug conjugate prepared by site-specific conjugation of PEGylated drug conjugate, for which the hydroxyl group of the payload is coupled with a polyethylene glycol (PEG) moiety, to a mono- or bispecific antibody.

### BACKGROUND OF INVENTION

Chemotherapy is one of the major treatment options for cancer therapy and is widely used in the clinic, but serious hurdles still remain such as drug resistance, systemic toxicity and narrow therapeutic window.

Antibody-drug conjugates (ADC) are anew class of targeted drugs. Up to 2021, there are twelve ADCs (exclusion of one fusion ADC) approved by FDA with more than 100 candidates of ADCs active in close to 300 clinical trials (research report by Beacon Targeted Therapies, Hanson Wade, UK). The biggest challenge today for ADC development is the requirement of dosing at very close to maximum tolerated dose (MTD) to show the benefit of the treatment, which results in a very narrow therapeutic window (Beck, A. et al., Nat. Rev. Drug Discov., 2017, 16, 315-337; Vankemmelbeke, M. et al., Ther. Deliv., 2016, 7, 141-144; Tolcher, A. W. et al., Ann. Oncol., 2016, 27, 2168-2172). The toxicity profiles of ADC found are often comparable with those of standard-of-care chemotherapeutics, with dose-limiting toxicities associated with cytotoxic warheads (Coats, S. et al. Clin. Cancer Res., 2019, 25, 5441-5448). As a result, 9 out of 12 FDA approved ADCs are required to carry black box warning labels due to severe adverse effect, which limits their applications in a variety of cancer indications, and over 80 ADCs discontinued in the course of clinical trials.

Furthermore, there are some inherited toxicities directly associated with the design and the structure of ADC. For instance, ADC toxicity could result from the off-target/off-tumor binding to Fc receptors (FcγRs) or lectin receptors (such as the mannose receptor) on normal cells (Donaghy, H. et al. MAbs, 2016, 8, 659-671), resulting in killing the FcγRs or mannose expressing cells due to the release of cytotoxic payload inside of the cells (Gorovits, B. et al. Cancer Immunol Immunother, 2013, 62, 217-223). Another Fc dependent toxicity results from the ADC aggregates, which can activate Fcγ receptors on immune cells, internalize via FcγRs, ultimately kill such target-negative cells (Aoyama, M. et al. Pharmaceutical Research, 2022, 39, 89-103).

Many natural or synthetic cytotoxic compounds could be used as payloads for ADC. The majority of ADCs in development or approved take advantage of an amino-bearing payload to form a stable carbamate bond linkage with a self-immolating spacer, which in turn links to a trigger molecule. The carbamate bond formed could ensure that payloads remain connected to the antibody during blood circulation. However, there is another large class of cytotoxic compounds, of which only available functional group for linking to an antibody is hydroxyl group. For this class of hydroxyl-bearing compounds, there are not as much research and development done as for cytotoxic amino-bearing compounds in the development of ADCs. The same strategy for attaching amino-bearing compound to the antibody via a self-immolating moiety, such as 4-aminophenyl alcohol (PAB), to form a stable carbamate linkage cannot be achieved for compounds with hydroxyl group as only available functional group for coupling, and the unstable carbonate linkage instead of carbamate bond will form, which could result in premature release of payload.

Antibody drugs including ADC are faced with several barriers that impact intratumoral distribution. The primary means of antibody transport inside tumors is based on diffusion, which is influenced by antibody size, binding affinity, tumor microenvironment, vascularization, and availability of targeted antigen (Xenaki, K. T. et al. Front Immunol, 2017, 8, 1287). The large size of antibody or ADC with molecule weight around 150 kd makes it difficult to extravasate the blood vessels to deep penetrate tumor tissue, while small size antibody fragments showed significantly increased tumor biodistribution (Li, Z. et al. MAbs, 2016, 8, 113-119). Binding site barrier (BSB) is another obstacle for antibody to penetrate tumor (Miao, L. et al. ACS Nano, 2016, 10, 9243-9258). Because the high affinity of the antibody to cellular target is the main reason for the binding site barrier, the strategy of co-administering a non-conjugated competitive antibody with ADC was adopted in a study. With this strategy, it was found that the effect of binding site barrier for ADC decreased and the ADC is more homogenously distributed in solid tumor (Evans, R. et al. Sci Rep., 2022, 12, 7677). However, this approach requires two antibody related products, which will increase the cost of treatment dramatically.

Therefore, there is a need for a novel ADC technology to address aforementioned issues.

### SUMMARY OF THE INVENTION

This invention addresses the unmet needs by providing non-immunogenic polymer modified antibody hydroxyl-bearing drug conjugates, prepared by site-specific conjugation of polymer modified (e.g. PEGylated) hydroxyl-bearing drug conjugate either to a mono-specific or multi-specific antibody fragment. The antibody fragment can be monovalent or multivalent for the antigens.

In one aspect, the invention provides a polymer antibody drug conjugate molecule of the Formula Ia

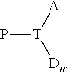

P can be a non-immunogenic polymer. T can be a multifunctional (e.g. trifunctional) small molecule linker moiety and have at least one functional group that is capable of site-specific conjugation to a mono-specific or multi-specific antibody or protein. A can be any mono-specific or multi-specific antibody or protein. D can be any hydroxyl-bearing cytotoxic molecule (n≥1), and each D can be the same or different.

In particular, an aspect of the invention provides a conjugate of Formula Ib:

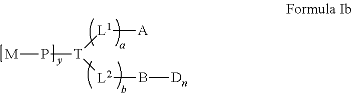

wherein
P can be a non-immunogenic polymer;
M can be H or a terminal capping group selected from C₁₋₅₀ alkyl and aryl, wherein one or more carbons of said alkyl are optionally replaced with a heteroatom;
y can be an integer selected from 1, 2, 3, 4, 5, 6, 7, 8, 9 and 10;
T can be a multi-functional linker having two or more functional groups, including but not limited to a trifunctional or tetrafunctional or any other cyclic or noncyclic multifunctional moiety (e.g. a lysine), wherein the linkage between T and (L¹)ₐ and the linkage between T and (L²)_{b} can be the same or different;
Each of L¹ and L² can be independently a bifunctional linker;
Each of a and b can be an integer selected from 0, 1, 2, 3, 4, 5, 6, 7, 8, 9 and 10;
B can be a branched linker, wherein each branch can comprise an extension spacer (optional), a trigger unit, one or more self-immolating spacer or any combination of such, wherein a trigger unit can be an amino acid sequence, a disulfide bond cleavable by an enzyme or a pH liable linker, or any cleavable bond that can release the hydroxyl-bearing drug D by certain cleavage mechanism;
A can be any mono-specific or multi-specific antibody or antigen binding protein, including an antibody fragment, a single chain antibody, a nanobody (single-domain antibody) or any antigen binding fragment, which can be monovalent or multivalent for the antigens;
D can be any cytotoxic hydroxyl-bearing small molecule or derivative thereof;
n can be an integer selected from 1, 2, 3, 4, 5, 6, 7, 8, 9, 10, 11, 12, 13, 14, 15, 16, 17, 18, 19 and 20.

Another aspect of the invention provides a conjugate of Formula Ic:

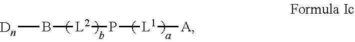

wherein each of the variables are as defined for Formula Ib.

In some embodiments, each branch of B comprises an extension spacer (optional), a trigger moiety, e.g. an amino acid sequence or a disulfide moiety or a carbohydrate moiety such as β-glucoronide or β-galactoside, connected to a hydroxyl-bearing drug D via one or more self-immolating spacer, cleavable by e.g. cathepsins B, plasmin, matrix metalloproteinases (MMPs), glutathione, thioredoxin family members (WCGH/PCK), thio reductase (Arunachalam, B. et. al. Proc. Nat. Acad. Sci. USA, 2000, 97, 745-750). Examples of one or two self-immolating spacers include but not limit to the following:

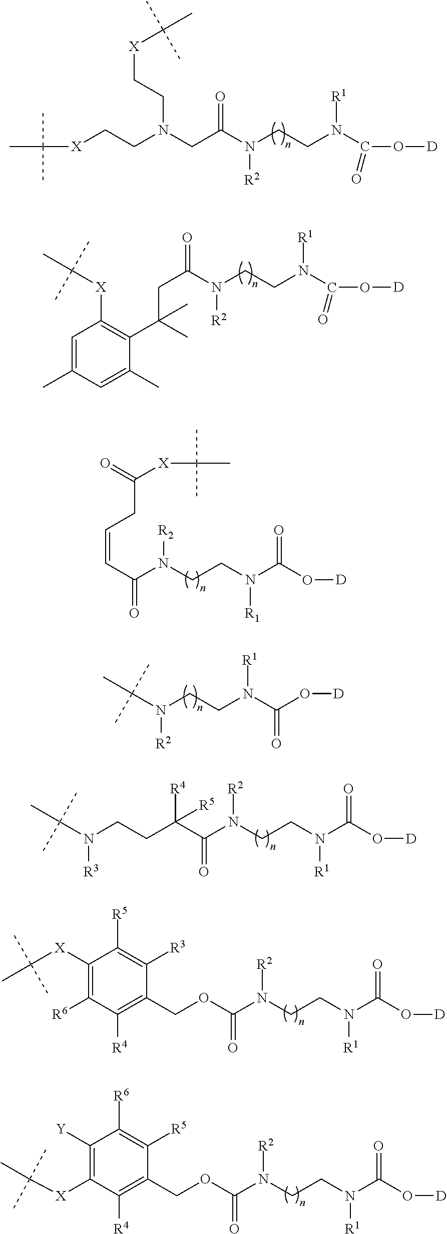

wherein n is 1 or 2; Y is a carbohydrate moiety; R¹, R², R³, R⁴, R⁵, R⁶ can be H, or C₁₋₁₀ alkyl or —(CH₂CH₂—O)₁₋₁₀—CH₃ or any combination thereof and X=O, S or N. In such embodiments, D can be any small molecule or peptide or derivative thereof containing active —OH functional group.

In some embodiments, A is a mono-specific antibody that is monovalent or bivalent for the antigens, e.g. a mono-specific single chain antibody that is monovalent or bivalent for the antigens.

In some embodiments, A is a multi-specific antibody, e.g. a bispecific single chain antibody.

In some embodiments, the two binding domains of the bispecific antibody bind to two of the same tumor associated antigen (TAA) molecules, but at two different epitopes, or bind to two different TAA molecule.

In a further embodiment, A is a single chain anti-PDL1 x anti-CD47 antibody that binds to PDL1 and CD47 expressed on cancer cells.

In another embodiment, A is a single chain anti-HER2(1) x anti-HER2(2) antibody that binds to HER2 expressed on cancer cells.

In still another embodiment, A is a single chain anti-cMet(1) x anti-cMet(2) antibody that binds to cMet expressed on cancer cells.

In some embodiments, the antibody has an amino acid sequence as shown in SEQ ID No. 1, SEQ ID No. 2, SEQ ID No. 3, SEQ ID No. 4, SEQ ID No. 5 or SEQ ID No. 6.

In some embodiments, the two binding domains of the single chain antibodies are linked via a linker, and wherein the linker can comprise a moiety such as cysteine or an unnatural amino acid residue for site-specific conjugation of the antibody to L¹.

In some embodiments, D can be selected from any hydroxyl-bearing DNA crosslinker agent, microtubule inhibitor, DNA alkylator, topoisomerase inhibitor, protein degraders, STING agonists or a combination thereof.

In some embodiments, D can be selected from, Dxd, SN38, calicheamicins, pyrrolobenzodiazepines, sibiromycin, tomaymycin, duocarmycins, neothramycins, DC-81, psymberin, vinca alkaloid, laulimalide, taxane, tubulysins, rhizoxin, discodermolide, taccalonolide A or B or AF or AJ, taccalonolide AI-epoxide, epothilone A and B, paclitaxel, docetaxel, doxorubicin, camptothecin, tafuramycin A, PNU-159682, uncialamycin, 0-amanitin, amatoxins, thailanstatins or any hydroxyl-bearing cytotoxic compounds or their analogs/derivates thereof, or a combination thereof.

In some embodiments, D is SN38 or Dxd (a potent topoisomerase I inhibitor), or duocarmycin (a DNA alkylator) or their analogs/derivatives, or a combination thereof.

In some embodiments, D is SN38 and is connected to a self-immolating spacer such as 4-aminobenzyl alcohol (PAB) via —NR′—(CH₂)ₙCH₂—NR²— (EDA), wherein n=1 or 2, R¹ or R² can be H, low MW alkyl or —(CH₂CH₂O)₁₋₁₀—CH₃ (1 to 10 PEG units); PAB is connected to a trigger moiety such as valine-citrulline.

In other embodiments, D is Dxd and is connected to a self-immolating spacer such as 4-aminobenzyl alcohol (PAB) via —NR1-(CH2)ₙCH2-NR2- (EDA), wherein n=1 or 2, R¹ or R² can be H, low MW alkyl or —(CH2CH2O)1-10-CH3 (1 to 10 PEG units); PAB is connected to a trigger moiety such as valine-citrulline.

In a further embodiment, D is duocarmycin DM and is connected to a self-immolating spacer such as 4-aminobenzyl alcohol (PAB) via —NR¹—(CH₂)ₙCH₂—NR²— (EDA), wherein n=1 or 2, R¹ or R² can be H, low MW alkyl or —(CH₂CH₂O)₁₋₁₀—CH₃ (1 to 10 PEG units); PAB is connected to a trigger moiety such as valine-citrulline.

In any of the above aspects and embodiments, the non-immunogenic polymer can be selected from the group consisting of polyethylene glycol (PEG), dextrans, carbohydrate polymers, polyalkylene oxide, polyvinyl alcohols, hydroxypropyl-methacrylamide (HPMA), and a co-polymer thereof Preferably, the non-immunogenic polymer is PEG, such as a branched PEG or a linear PEG, wherein the PEG can be linked to the multifunctional moiety T either through a permanent bond or a cleavable bond. The total molecule weight of the PEG can be ranged from 3000 to 100,000 Daltons, e.g., 5000 to 80,000, 10,000 to 60,000, 10000 to 30000, or 20,000 to 40,000 Daltons, e.g. about 10000, 20000, 30000 or 40000 Daltons.

Functional group for site-specific conjugation that forms linkage between (L¹)ₐ and protein A can be selected from the group consisting of thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid, iodine and the like.

In some embodiments, one of (L¹)ₐ can comprise a linkage formed from azide and alkyne or from maleimide and thiol. In some embodiments, the alkyne can be dibenzocyclooctyl (DBCO).

In some embodiments, T can be lysine, aspartic acid, glutamic acid, serine, tyrosine, or any other molecules with trifunctional groups, P can be PEG, and y can be 1, while the alkyne can be dibenzocyclooctyl (DBCO).

In some embodiments, A can be derived from an azide tagged mono- or multi-specific antibody or antigen binding protein including antibody fragment, a single chain antibody, a nanobody or any antigen binding fragment thereof, or a combination thereof, wherein the azide can be conjugated to an alkyne in the respective (L¹)ₐ. In other embodiments, A can be derived from a thiol tagged mono- or multi-specific antibody or antigen binding protein including an antibody fragment, a single chain antibody, a nanobody or any antigen binding fragment thereof, or a combination thereof, wherein the thiol can be conjugated to a maleimide in the respective (L¹)ₐ.

The above-described antibody drug conjugate can be made according to a method comprising: (i) preparing a non-immunogenic polymer drug conjugate with a terminal functional group that is capable of site-specific conjugation to an antibody or a protein or its modified form; and (ii) site-specific conjugating the non-immunogenic polymer drug conjugate to an antibody or a protein or its modified structure to form a compound of Formula Ia, Ib or Ic. In some examples, the antibody or protein can be modified with a small molecule linker before the conjugation.

The invention also provides a pharmaceutical formulation comprising the above-described antibody hydroxyl-bearing drug conjugate e.g. PEGylated mono- or bispecific single chain antibody hydroxyl-bearing drug conjugate that is monovalent or multivalent for the antigens and a pharmaceutically acceptable carrier.

The invention further provides a method of treating a disease in a subject in need thereof comprising administering an effective amount of the above-described antibody hydroxyl-bearing drug conjugate e.g. PEGylated mono- or bispecific single chain antibody drug conjugate that is monovalent or multivalent for the antigens.

The details of one or more embodiments of the invention are set forth in the description below. Other features, objectives, and advantages of the invention will be apparent from the drawing, description and claims.

The present disclosure further provides following embodiments.

Embodiment 1. A compound of the Formula (Ib)

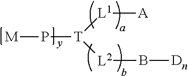

wherein
P is a non-immunogenic polymer;
M is H or a terminal capping group selected from C₁₋₅₀ alkyl and aryl, wherein one or more carbons of said alkyl are optionally replaced with a heteroatom;
y is an integer selected from 1 to 10, e.g. 1, 2, 3, 4, 5, 6, 7, 8, 9 or 10;
A is an antibody or an antigen binding fragment thereof;
T is a multifunctional small molecule linker moiety;
each of L¹ and L² is independently a hetero- or homobifunctional linker;
each of a and b is an integer selected from 0-10, e.g. 0, 1, 2, 3, 4, 5, 6, 7, 8, 9, 10;
B is a branched linker, wherein each branch has an amino acid sequence or a disulfide bond or carbohydrate moiety or a cleavable bond linked to one or more self-immolating spacer, wherein cleavage of the amino acid sequence or disulfide bond or carbohydrate moiety or cleavable bond by an enzyme triggers self-immolating mechanism to release hydroxyl-bearing D or its derivatives;
each of D is independently a cytotoxic hydroxyl-bearing small molecule or peptide; and n is an integer selected from 1-25, e.g. 1-20, 1-15, 1-10, 1-5, 5-25, 5-20, 5-15, 5-10, 10-25, 10-20, 10-15, 15-25, 15-20 or 20-25, e.g. 1, 2, 3, 4, 5, 6, 7, 8, 9, 10, 11, 12, 13, 14, 15, 16, 17, 18, 19, 20, 21, 22, 23, 24, 25.

Embodiment 2. The compound of embodiment 1, wherein T is a tri-functional linker derived from a molecule with three functional groups independently selected from hydroxyl, amino, hydrazinyl, azide, alkene, alkyne, carboxyl (aldehyde, ketone, ester, carboxylic acid, anhydride, acyl halide), thiol, disulfide, nitrile, epoxide, imine, nitro and halide, and wherein the linkage between T and (L¹)ₐ and the linkage between T and (L²)_{b} are the same or different.

Embodiment 3. The compound of embodiment 2, wherein T is lysine, aspartic acid, glutamic acid, serine, tyrosine, or any other cyclic or noncyclic molecules with trifunctional groups or its derivative.

Embodiment 4. The compound of any of embodiments 1-3, wherein one of the functional group at the linker terminal of (L¹)ₐ is capable of site-specific conjugation with A, and is selected from the group consisting of thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and iodine.

Embodiment 5. The compound of any of embodiments 1-4, wherein the antibody is a mono-specific or multi-specific full length antibody, a single chain antibody, a nanobody (single-domain antibody), or an antigen binding domain thereof.

Embodiment 6. The compound of any one of embodiments 1-5, wherein the antibody is a mono-specific single chain antibody.

Embodiment 7. The compound of embodiment 6, wherein the mono-specific single chain antibody binds to a tumor associated antigen (TAA) such as Her2, cMet, PDL1, or CD47.

Embodiment 8. The compound of embodiment 7, wherein the mono-specific single chain antibody has two binding domains binding to Her2.

Embodiment 9. The compound of embodiment 8, wherein the mono-specific single chain antibody has an amino acid sequence as shown in SEQ ID No. 3.

Embodiment 10. The compound of any one of embodiments 1-5, wherein the antibody is a bispecific antibody, e.g. a bispecific single chain antibody.

Embodiment 11. The compound of embodiment 10, wherein the two binding domains of the bispecific antibody bind to the same tumor associated antigen (TAA), bind to two different TAAs, or bind to a TAA and an antigen expressed on T cells (e.g. a component of T cell receptor) or NK cells.

Embodiment 12. The compound of embodiment 11, wherein the antibody is an anti-PDL1 x anti-CD47 single chain bispecific antibody or an anti-HER2(1) x anti-HER2(2) single chain bispecific antibody or an anti-cMet(1) x anti-cMet(2) single chain bispecific antibody.

Embodiment 13. The compound of embodiment 12, wherein the antibody has an amino acid sequence as shown in SEQ ID No. 1 or SEQ ID No. 2 or SEQ ID No. 6.

Embodiment 14. The compound of any of embodiments 6-9, wherein the two binding domains of the mono-specific single chain antibody are linked via a linker, and wherein the linker comprises a moiety such as cysteine or an unnatural amino acid residue for site-specific conjugation of the antibody to (L¹)ₐ.

Embodiment 15. The compound of any of embodiments 10-13, wherein the two binding domains of the bispecific single chain antibody are linked via a linker, and wherein the linker comprises a moiety such as cysteine or an unnatural amino acid residue for site-specific conjugation of the antibody to (L¹)ₐ.

Embodiment 16. The compound of any of embodiments 14-15, wherein the unnatural amino acid residue is selected from the group consisting of genetically-encoded alkene lysines (such as N6-(hex-5-enoyl)-L-ly sine), 2-Amino-8-oxononanoic acid, m or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino)hexanoic acid, azidohomoalanine, pyrrolysine analogue N6-((prop-2-yn-1-yloxy)carbonyl)-L-lysine, (S)-2-amino-6-pent-4-ynamidohexanoic acid, (S)-2-amino-6-((prop-2-ynyloxy)carbonylamino)hexanoic acid, (S)-2-amino-6-((2-azidoethoxy)carbonylamino)hexanoic acid, β-azidophenylalanine, para-azidophenylalanine, Nε-Acryloyl-l-lysine, Nε-5-norbornene-2-yloxycarbonyl-l-lysine, N-ε-(cyclooct-2-yn-1-yloxy)carbonyl)-L-lysine, N-ε-(2-(cyclooct-2-yn-1-yloxy)ethyl) carbonyl-L-lysine, and genetically encoded tetrazine amino acid (such as 4-(6-methyl-s-tetrazin-3-yl)aminophenylalanine).

Embodiment 17. The compound of any one of embodiments 1-16, wherein D is selected from a DNA crosslinker agent, a microtubule inhibitor, a DNA alkylator, a topoisomerase inhibitor, protein degrader, STING agonist or a combination thereof.

Embodiment 18. The compound of embodiments 17, wherein D is selected from Dxd, SN38, calicheamicins, pyrrolobenzodiazepines, sibiromycin, tomaymycin, duocarmycins, neothramycins, DC-81, psymberin, vinca alkaloid, laulimalide, taxane, tubulysins, rhizoxin, discodermolide, taccalonolide A or B or AF or AJ, taccalonolide AI-epoxide, epothilone A and B, paclitaxel, docetaxel, doxorubicin, camptothecin, tafuramycin A, PNU-159682, uncialamycin, β-amanitin, amatoxins, thailanstatins or any hydroxyl-bearing cytotoxic compounds or their analogs/derivates thereof, or a combination thereof.

Embodiment 19. The compound of any one of embodiments 1-18, wherein the non-immunogenic polymer is polyethylene glycol (PEG).

Embodiment 20. The compound of embodiment 19 wherein the PEG is a liner PEG or a branched PEG.

Embodiment 21. The compound of any one of embodiment 19-20, wherein at least one terminal of the polyethylene glycol is capped with methyl or a low molecule weight alkyl.

Embodiment 22. The compound of any of embodiment 19-21, wherein a total molecule weight of the PEG is from 3000 to 100000 Dalton.

Embodiment 23. The compound of any one of embodiments 19-22, wherein the PEG is linked to the trifunctional or tetrafunctional or any other cyclic or noncyclic multifunctional moiety T (e.g. a lysine) through a permanent bond or a cleavable bond.

Embodiment 24. A compound of the Formula (Ic)

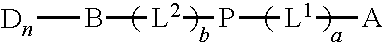

wherein
P is a liner PEG;
A is an antibody or an antigen binding fragment thereof;
each of L¹ and L² is independently a bifunctional linker;
each of a and b is an integer selected from 0-10, e.g. 0, 1, 2, 3, 4, 5, 6, 7, 8, 9, 10;
B is a branched linker, wherein each branch has an amino acid sequence or a disulfide bond or carbohydrate moiety or a cleavable bond linked to one or more self-immolating spacer, wherein cleavage of the amino acid sequence or disulfide bond or carbohydrate moiety by an enzyme, e.g. cathepsins B, plasmin, matrix metalloproteinases (MMPs), glutathione, thioredoxin family members (WCGH/PCK), thio reductase, triggers self-immolating mechanism to release D or its derivatives;
each of D is independently a cytotoxic hydroxyl-bearing small molecule or peptide;
n is an integer selected from 1-25, e.g. 1-20, 1-15, 1-10, 1-5, 5-25, 5-20, 5-15, 5-10, 10-25, 10-20, 10-15, 15-25, 15-20 or 20-25, e.g. 1, 2, 3, 4, 5, 6, 7, 8, 9, 10, 11, 12, 13, 14, 15, 16, 17, 18, 19, 20, 21, 22, 23, 24, 25.

Embodiment 25. The compound of embodiment 24, wherein one of the functional group at the linker terminal of (L¹)ₐ is capable of site-specific conjugation with A, and is selected from the group consisting of thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and iodine.

Embodiment 26. The compound of any of embodiments 24-25, wherein the antibody is a mono-specific or multi-specific full length antibody, a single chain antibody, a nanobody (single-domain antibody), or an antigen binding domain thereof.

Embodiment 27. The compound of embodiment 26, wherein the antibody is a mono-specific single chain antibody, optionally wherein the mono-specific single chain antibody binds to a tumor associated antigen (TAA) such as Her2, cMet, PDL1 or CD47.

Embodiment 28. The compound of embodiment 27, wherein the mono-specific single chain antibody has two binding domains binding to Her2.

Embodiment 29. The compound of embodiment 28, wherein the mono-specific single chain antibody has an amino acid sequence as shown in SEQ ID No. 3.

Embodiment 30. The compound of embodiment 26, wherein the antibody is a bispecific antibody, e.g. a bispecific single chain antibody.

Embodiment 31. The compound of embodiment 30, wherein the two binding domains of the bispecific antibody bind to the same tumor associated antigen (TAA), bind to two different TAAs, or bind to a TAA and an antigen expressed on T cells (e.g. a component of T cell receptor) or NK cells.

Embodiment 32. The compound of embodiment 30, wherein the antibody is an anti-PDL1 x anti-CD47 single chain bispecific antibody or an anti-HER2(1) x anti-HER2(2) single chain bispecific antibody or an anti-cMet(1) x anti-cMet(2) single chain bispecific antibody.

Embodiment 33. The compound of embodiment 32, wherein the antibody has an amino acid sequence as shown in SEQ ID No. 1 or SEQ ID No. 2 or SEQ ID No. 6.

Embodiment 34. The compound of any of embodiments 27-29, wherein the two binding domains of the mono-specific single chain antibody are linked via a linker, and wherein the linker comprises a moiety such as cysteine or an unnatural amino acid residue for site-specific conjugation of the antibody to (L¹)ₐ.

Embodiment 35. The compound of any of embodiments 30-33, wherein the two binding domains of the bispecific single chain antibody are linked via a linker, and wherein the linker comprises a moiety such as cysteine or an unnatural amino acid residue for site-specific conjugation of the antibody to (L¹)ₐ.

Embodiment 36. The compound of any of embodiments 34-35, wherein the unnatural amino acid residue is selected from the group consisting of nongenetically-encoded alkene lysines (such as N6-(hex-5-enoyl)-L-lysine), 2-amino-8-oxononanoic acid, m or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino)hexanoic acid, azidohomoalanine, pyrrolysine analogue N6-((prop-2-yn-1-yloxy)carbonyl)-L-lysine, (S)-2-amino-6-pent-4-ynamidohexanoic acid, (S)-2-amino-6-((prop-2-ynyloxy)carbonylamino)hexanoic acid, (S)-2-amino-6-((2-azidoethoxy)carbonylamino)hexanoic acid, p-azidophenylalanine, para-azidophenylalanine, Nε-Acryloyl-l-lysine, Nε-5-norbornene-2-yloxycarbonyl-l-lysine, N-ε-(cyclooct-2-yn-1-yloxy)carbonyl)-L-lysine, N-ε-(2-(cyclooct-2-yn-1-yloxy)ethyl) carbonyl-L-lysine, and genetically encoded tetrazine amino acid (such as 4-(6-methyl-s-tetrazin-3-yl)aminophenylalanine).

Embodiment 37. The compound of any one of embodiments 24-36, wherein D is selected from a DNA crosslinker agent, a microtubule inhibitor, a DNA alkylator, a topoisomerase inhibitor, STING agonist, protein degrader or a combination thereof.

Embodiment 38. The compound of any one of embodiments 37, wherein hydroxyl-bearing D is selected from Dxd, SN38, calicheamicins, pyrrolobenzodiazepines, sibiromycin, tomaymycin, duocarmycins, neothramycins, DC-81, psymberin, vinca alkaloid, laulimalide, taxane, tubulysins, rhizoxin, discodermolide, taccalonolide A or B or AF or AJ, taccalonolide AI-epoxide, epothilone A and B, paclitaxel, docetaxel, doxorubicin, camptothecin, tafuramycin A, PNU-159682, uncialamycin, 0-amanitin, amatoxins, thailanstatins or any hydroxyl-bearing cytotoxic compounds or their analogs/derivates thereof, or a combination thereof.

Embodiment 39. The compound of any of embodiment 24-38, wherein a total molecule weight of the PEG is from 3000 to 100000 Dalton.

Embodiment 40. The compound of any of embodiment 1-39, wherein each of L¹ and L² is independently selected from the group consisting of:
—(CH₂)ₐXY(CH₂)_{b}—,
—X(CH₂)ₐO(CH₂CH₂O)_{c}(CH₂)_{b}Y—,
—(CH₂)ₐheterocyclyl-,
—(CH₂)ₐX—,
—X(CH₂)ₐY—,
—W₁—(CH₂)ₐC(O)NR₁(CH₂)_{b}O(CH₂CH₂O)_{c}(CH₂)_{d}C(O)—,
—C(O)(CH₂)ₐO(CH₂CH₂O)_{b}(CH₂)_{c}W₂C(O)(CH₂)_{d}NR₁—, and
—W₃—(CH₂)ₐC(O)NR₁(CH₂)_{b}O(CH₂CH₂O)_{c}(CH₂)_{d}W₂C(O)(CH₂)ₑC(O)—,
wherein each of a, b, c, d and e is independently an integer selected from 0 to 25, e.g. 0-20, 0-15, 0-10, 0-5, 5-25, 5-20, 5-15, 5-10, 10-25, 10-20, 10-15, 15-25, 15-20 or 20-25, e.g. 0, 1, 2, 3, 4, 5, 6, 7, 8, 9, 10, 11, 12, 13, 14, 15, 16, 17, 18, 19, 20, 21, 22, 23, 24, 25; each of X and Y is independently selected from C(═O), NR₂, S, O, N₃, CR₃R₄, a DBCO-based moiety or Null; each of R₁, R₂, R₃ and R₄ independently represents hydrogen, C₁₋₁₀ alkyl or (CH₂)₁₋₁₀C(═O); W₁ and/or W₃ is derived from a maleimido-based moiety and W₂ represents a triazolyl or a tetrazolyl containing group; and the heterocyclyl group is selected from a maleimido-derived moiety or a tetrazolyl-based or a triazolyl-based moiety.

Embodiment 41. The compound of any of embodiments 1-39, wherein each of L¹ and L² is independently selected from:

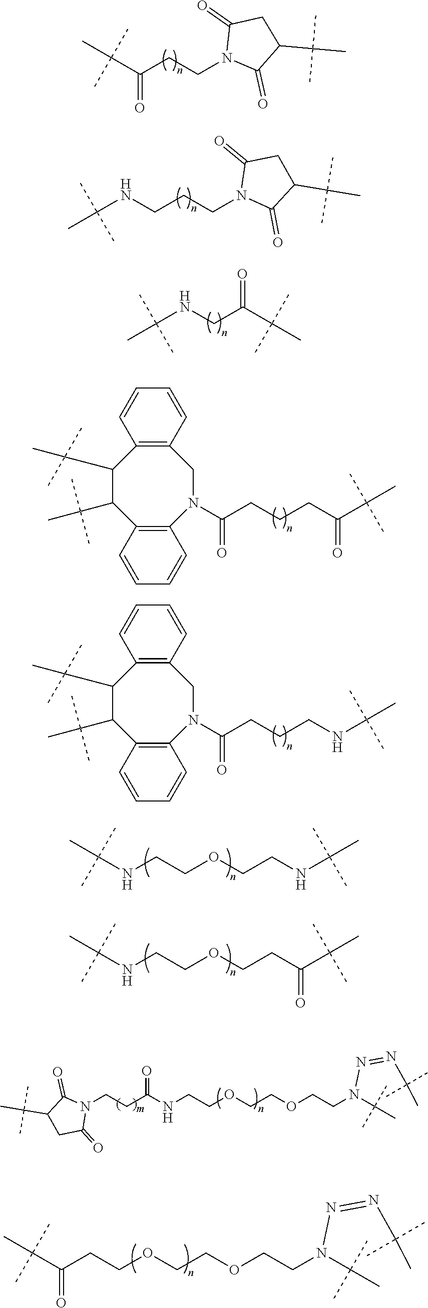

Wherein each of n and m is independently an integer selected from 0 to 20, e.g. 0-15, 0-10, 0-5, 5-20, 5-15, 5-10, 10-20, 10-15, or 15-20, e.g. 0, 1, 2, 3, 4, 5, 6, 7, 8, 9, 10, 11, 12, 13, 14, 15, 16, 17, 18, 19, or 20.

Embodiment 42. The compound of any of embodiments 1-41, wherein the branched linker B comprise an extension spacer (optional), a trigger unit, one or more self-immolating spacer or any combination thereof, optionally wherein the trigger unit is an amino acid sequence or a disulfide bond or a β-glucoronide or β-galactoside trigger moiety cleavable by an enzyme such as cathepsin B, plasmin, matrix metalloproteinases (MMPs), β-glucuronidases, β-galactosidases, glutathione, thioredoxin family members (WCGH/PCK) or thio reductase.

Embodiment 43. The compound of embodiment 42, wherein the branched linker B is selected from

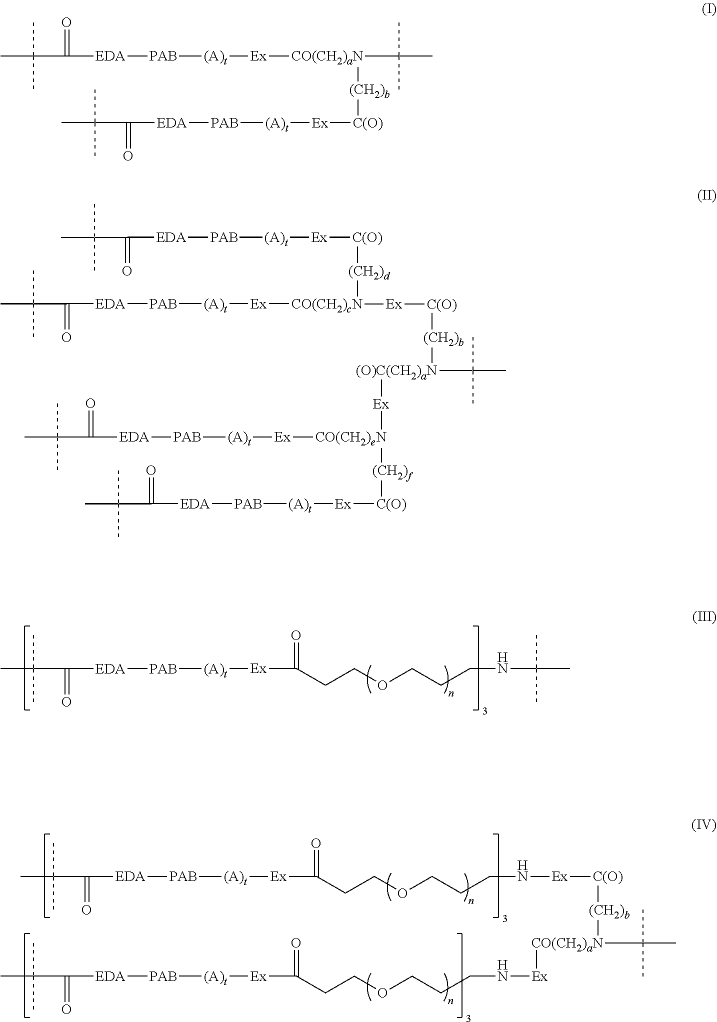

wherein:
each of a, b, c, d, e and f is independently an integer selected from 1-25 e.g. 1-20, 1-15, 1-10, 1-5, 5-25, 5-20, 5-15, 5-10, 10-25, 10-20, 10-15, 15-25, 15-20 or 20-25, e.g. 1, 2, 3, 4, 5, 6, 7, 8, 9, 10, 11, 12, 13, 14, 15, 16, 17, 18, 19, 20, 21, 22, 23, 24, 25;
n is an integer selected from 1 to 10;
(A)ₜ is a trigger unit of amino acid sequence such as Val-Cit, Val-Ala, Val-Lys, Phe-Lys, Phe-Cit, Phe-Arg, Phe-Ala, Ala-Lys, Leu-Cit, Ile-Cit, Trp-Cit, D-Phe-Phe-Lys, Phe-Phe-Lys, Gly-Phe-Lys, Gly-Phe-Leu-Gly, Gly-Gly-Phe-Gly or Ala-Leu-Ala-Leu;
PAB is para-aminobenzyl alcohol;
EDA is —NR¹(CH₂)ₘNR²—, wherein m is 2 or 3, each of R¹ and R² is independently selected from H, a low molecule weight alkyl or —(CH₂CH₂O)ₜ—CH₃, wherein 1 is an integer selected from 1-10;
each of Ex is an extension spacer comprising a linker chain that is independently selected from:
—NR¹(CH₂)ₓO(CH₂CH₂O)_{y}(CH₂)_{z}C(O)—,
—C(O)(CH₂)ₓNR¹—,
—NR¹(CH₂)ₓO(CH₂CH₂O)_{y}(CH₂)_{z}NR²—,
—NR¹(CH₂)ₓNR²,
—NR¹(CH₂)ₓO(CH₂CH₂O)_{y}(CH₂)_{z}O—,
O(CH₂)ₓNR¹—,
—C(O)(CH₂)ₓO—,
—O(CH₂)ₓO(CH₂CH₂O)_{y}(CH₂)_{z}C(O)—,
—C(O)(CH₂)ₓO(CH₂CH₂O)_{y}(CH₂)_{z}C(O)—,
—C(O)(CH₂)ₓC(O)—,
or Null,
wherein each of x, y, and z is independently an integer selected from 0 to 25, e.g. 0-20, 0-15, 0-10, 0-5, 5-25, 5-20, 5-15, 5-10, 10-25, 10-20, 10-15, 15-25, 15-20 or 20-25, e.g. 0, 1, 2, 3, 4, 5, 6, 7, 8, 9, 10, 11, 12, 13, 14, 15, 16, 17, 18, 19, 20, 21, 22, 23, 24, 25; and each of R¹ and R²independently represents hydrogen or a C₁₋₁₀ alkyl group.

Embodiment 44. The compound of any of embodiments 1-41, wherein the branched linker B is selected from

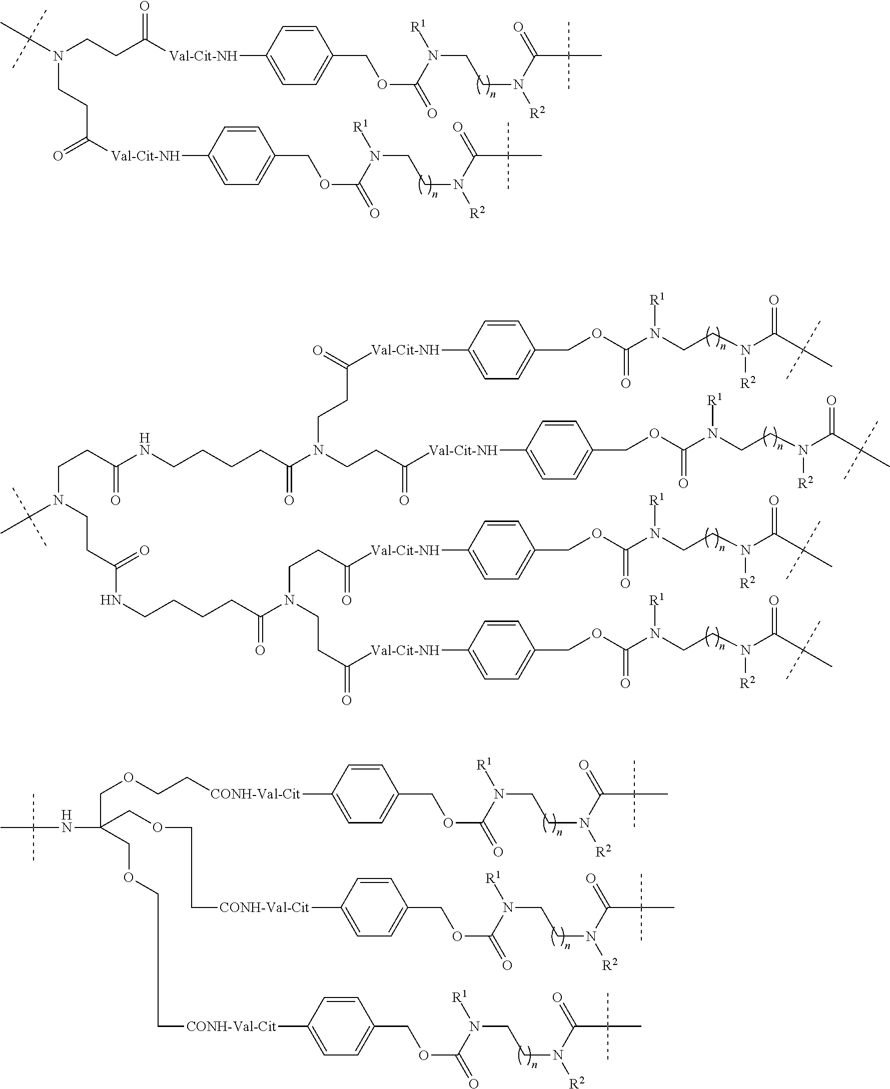

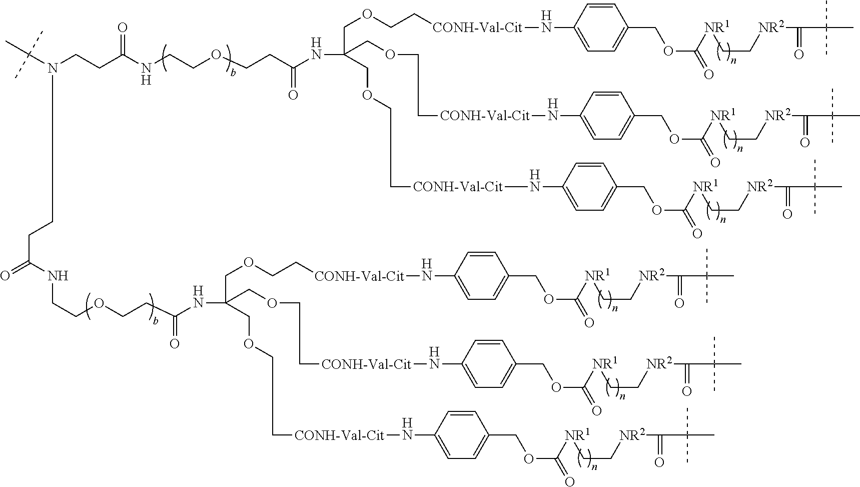

wherein n=1 or 2: b is an integer selected from 1 to 10: each of R¹ and R² is independently selected from H, a low molecule weight alkyl or —(CH₂CH₂O)ₘ—CH₃, wherein m=1-10.

Embodiment 45. The compound of embodiment 1 selected from the formula:

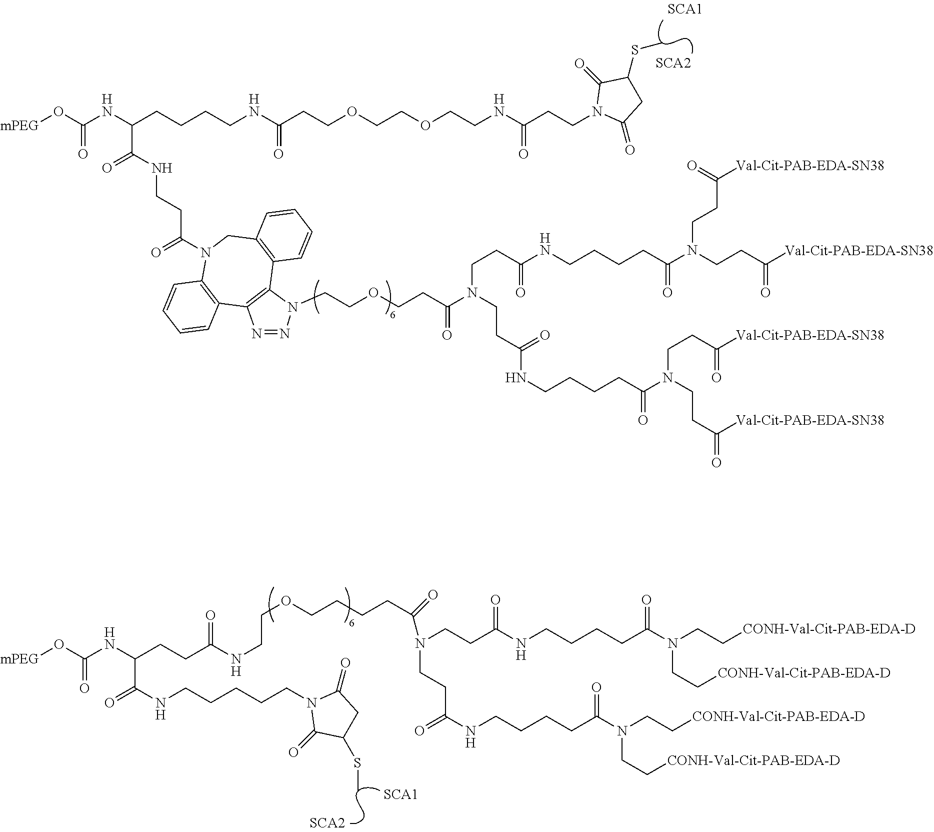

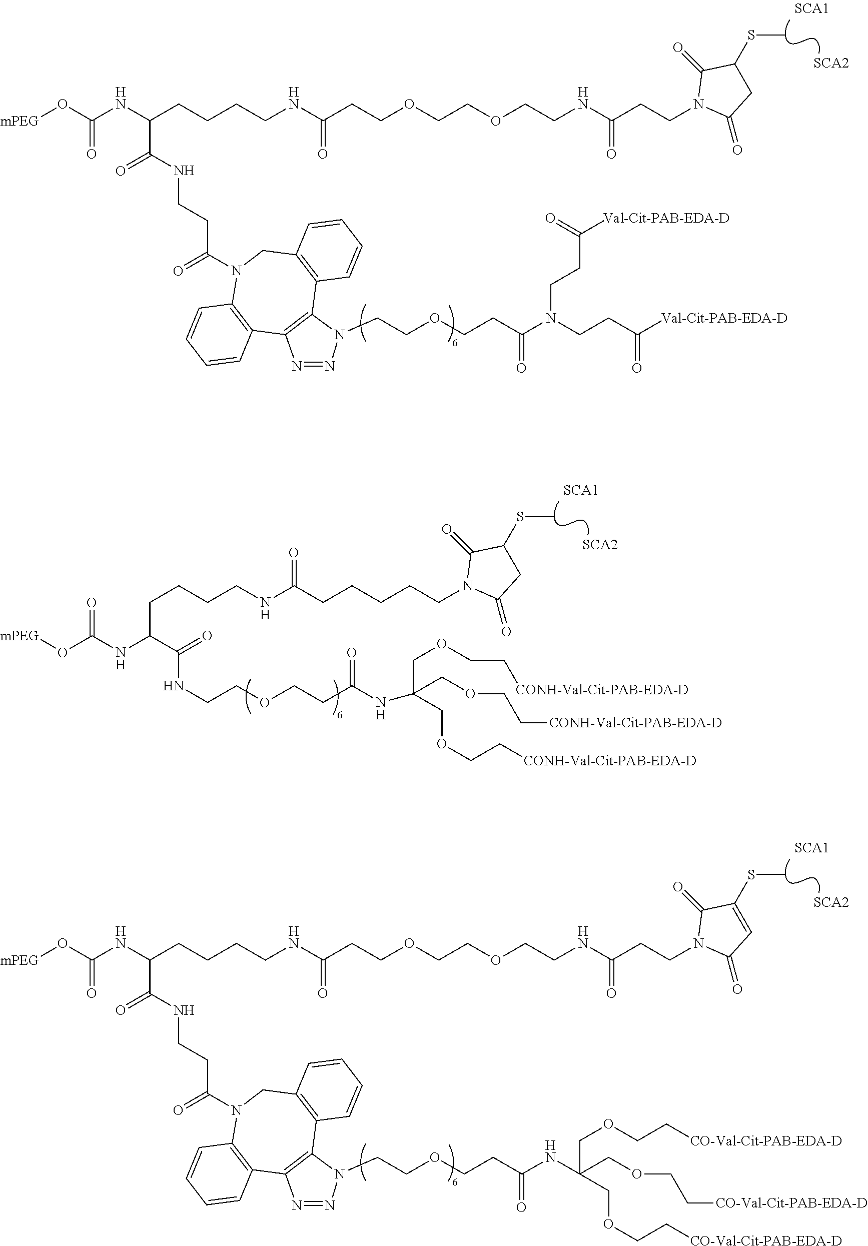

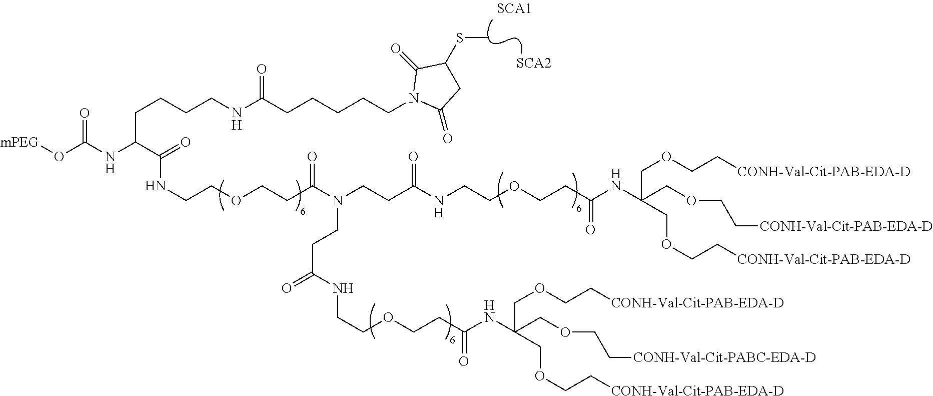

or a pharmaceutically acceptable salt thereof,
wherein: SCA1 and SCA2 are an anti-PDL1 and anti-CD47 single chain antibody or anti-HER2(1) and anti-HER2(2) single chain antibody or anti-cMet(1) and anti-cMet(2) single chain antibody, preferably having the amino acid as shown in SEQ ID No. 1 or SEQ ID No. 2 or SEQ ID No. 6; and the mPEG has a total molecular weight from 3000 to 100000 Dalton, e.g. 10000-40000 Daltons;

### Val-Cit-PAB-EDA-D is

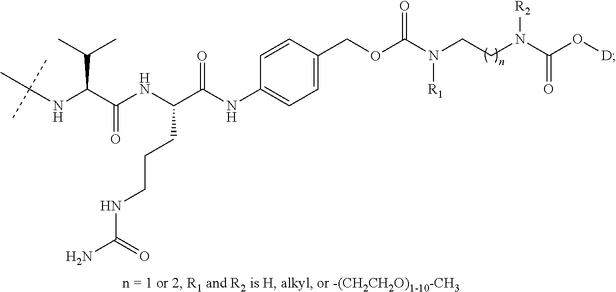

### D is

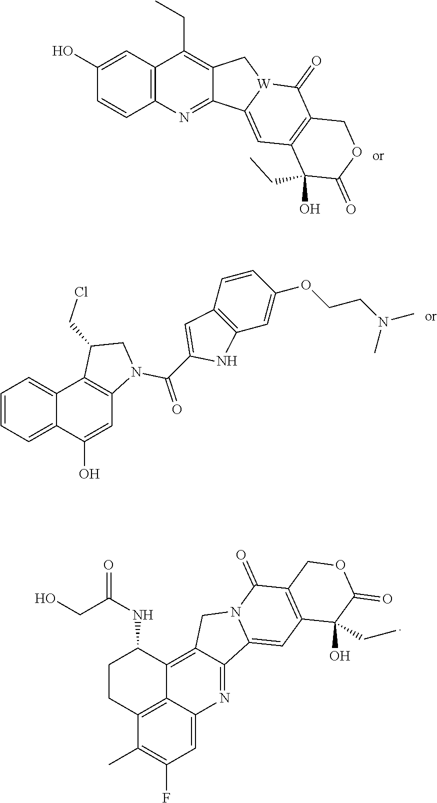

Embodiment 46. The compound of embodiment 24 selected from the formula:

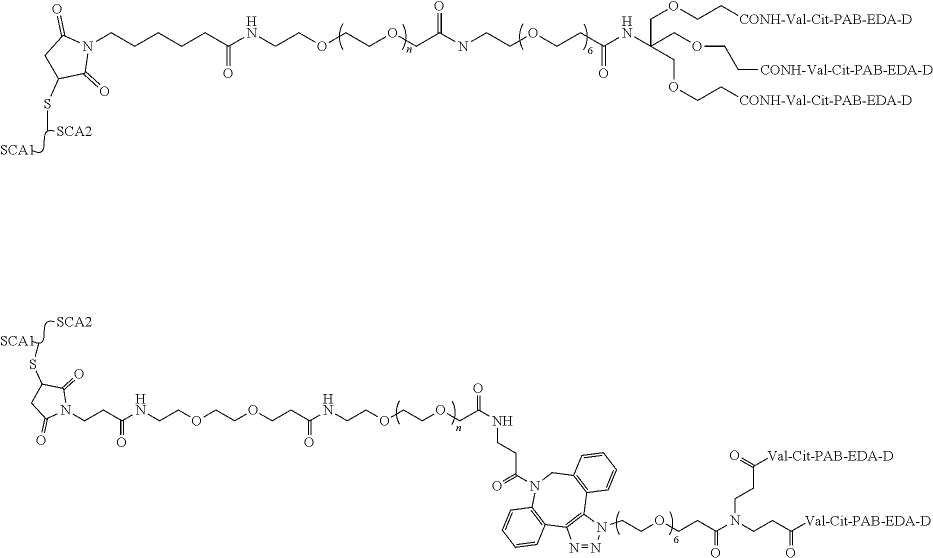

or a pharmaceutically acceptable salt thereof;
wherein: SCA1 and SCA2 are an anti-PDL1 and anti-CD47 single chain antibody or anti-HER2(1) and anti-HER2(2) single chain antibody or anti-cMet(1) and anti-cMet(2) single chain antibody, preferably having the amino acid as shown in SEQ ID No. 1 or SEQ ID No. 2 or SEQ ID No. 6;
n is an integer selected from 110 to 1800, preferably n is an integer selected from 220 to 910, or preferably wherein the total molecular weight of PEG is 10000-40000 Daltons, e.g. about 10000, 20000, 30000 or 40000 Daltons;

### Val-Cit-PAB-EDA-D is

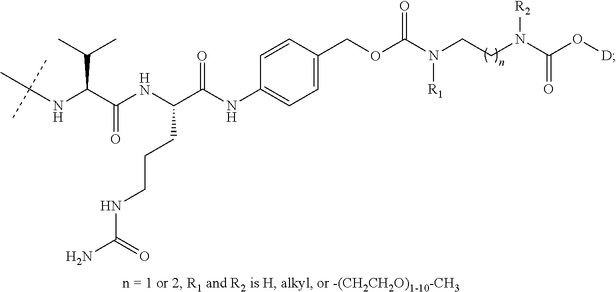

### D is

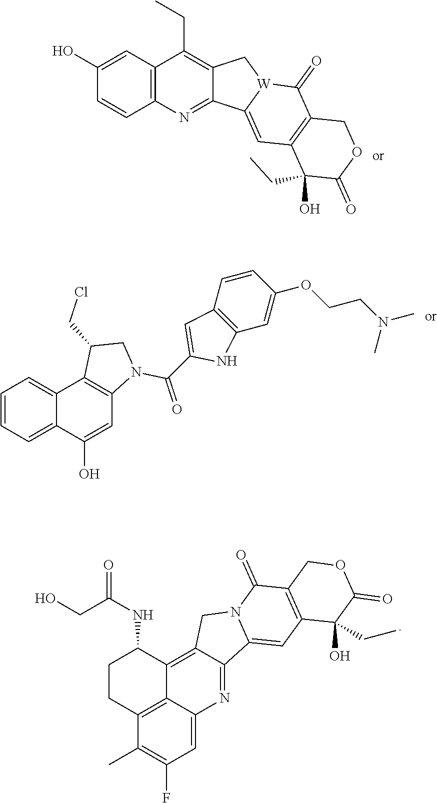

Embodiment 47. A method of preparing a compound of any one of embodiments 1-46, comprising:
a) a step of preparation of the non-immunogenic polymer modified (e.g. PEGylated) hydroxyl-bearing drug conjugate with a free functional group for site-specific conjugation;
b) a step of site-specific conjugation of the non-immunogenic polymer modified (e.g. PEGylated) hydroxyl-bearing drug conjugate to an antibody to provide a compound of the Formula (Ib) or (Ic).

Embodiment 48. A pharmaceutical formulation comprising an effective amount of the compound of any one of embodiments 1-46 and a pharmaceutically acceptable salt, carrier or excipient.

Embodiment 49. A compound of any one of embodiments 1 to 46 for use in the treatment of a cancer selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer head and neck cancer and endometrial cancer.

Embodiment 50. A compound of any one of embodiments 1 to 46 for use in combination with an effective amount of another anticancer agent or immunosuppressant agent in the treatment of a cancer selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer head and neck cancer and endometrial cancer.

Embodiment 51. A method of treating a cancer in a subject, comprising administrating to the subject an effective amount of the compound of any one of embodiments 1 to 46, wherein the cancer is selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer head and neck cancer and endometrial cancer.

Embodiment 52. The method of embodiment 51, wherein the method further comprises administering to the subject an effective amount of another anticancer agent or immunosuppressant agent.

Embodiment 53. Use of the compound of any one of embodiments 1 to 46 in the manufacture of a medicament for treating a cancer in a subject, wherein the cancer is selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer head and neck cancer and endometrial cancer.

Embodiment 54. The use of embodiment 53, wherein the compound is combined with another anticancer agent or immunosuppressant agent.

Embodiment 55. Use of the compound of any one of embodiments 1 to 46 and another anticancer agent or immunosuppressant agent in the manufacture of a medicament for treating a cancer in a subject, wherein the cancer is selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer head and neck cancer and endometrial cancer.

### DESCRIPTION OF THE DRAWINGS

FIG. 1 schematically illustrates a reaction scheme of preparing compound Fmoc-Val-Cit-PAB-PNP (5) described in example 1.

FIG. 2 schematically illustrates a reaction scheme of preparing compound Val-Cit-PAB-DEA-SN38 (10) described in example 1.

FIG. 3 schematically illustrates an alternative reaction scheme of preparing compound Val-Cit-PAB-DEA-SN38 (10) described in example 1.

FIG. 4 schematically illustrates a reaction scheme of preparing branched intermediate compound 16 described in example 1.

FIG. 5 schematically illustrates a reaction scheme of preparing compound 30kmPEG-Lys(Mal)-3(Val-Cit-PAB-DEA-SN38) (22) described in example 1.

FIG. 6 schematically illustrates a reaction scheme of preparing compound 30kmPEG-Lys(Mal)-6(Val-Cit-PAB-DEA-SN38) (28) in example 2.

FIG. 7 schematically illustrates a reaction scheme of preparing compound Val-Cit-PAB-DEA-Duo-DM (33) in example 3.

FIG. 8 schematically illustrates a reaction scheme of preparing compound branched intermediate compound (40) in example 3.

FIG. 9 schematically illustrates an alternative reaction scheme of preparing compound branched intermediate compound (40) in example 3.

FIG. 10 schematically illustrates a reaction scheme of preparing compound 20kmPEG-Glu(Mal)-4(Val-Cit-PAB-DEA-Duo-DM) (46) in example 3.

FIG. 11 schematically illustrates a reaction scheme of preparing compound branched intermediate Compound (48) in example 4.

FIG. 12 schematically illustrates a reaction scheme of preparing compound Mal-20kPEG-3(Val-Cit-PAB-DEA-Duo-DM) (51) in example 4.

FIG. 13 schematically illustrates a reaction scheme of preparing compound 30kmPEG-(SCAPDL1xSCACD47)-3(Val-Cit-PAB-DEA-SN38) (53) in example 6.

FIG. 14 schematically illustrates a reaction scheme of preparing compound 30kmPEG-(SCAPDL1xSCACD47)-6(Val-Cit-PAB-DEA-SN38) (54) in example 7.

FIG. 15 schematically illustrates a reaction scheme of preparing compound 20kmPEG-(SCAPDL1xSCACD47)-4(Val-Cit-PAB-DEA-Duo-DM) (55) in example 8.

FIG. 16 schematically illustrates a reaction scheme of preparing compound SCAPDL1xSCACD47-20kPEG-3(Val-Cit-PAB-DEA-Duo-DM) (56) in example 9.

FIG. 17 schematically illustrates a reaction scheme of preparing compound branched intermediate compound (58) in example 10.

FIG. 18 schematically illustrates a reaction scheme of preparing compound branched intermediate compound N₃-PEG₆-3(Val-Cit-PAB-DEA-SN38) (62) in example 11.

FIG. 19 schematically illustrates a reaction scheme of preparing compound branched intermediate compound N₃-PEG₆-2(Val-Cit-PAB-DEA-SN38) (65) and N₃-PEG₆-4(Val-Cit-PAB-DEA-SN38) (66) in example 12.

FIG. 20 schematically illustrates a reaction scheme of preparing 30kmPEG-Lys(PEG₂-Mal)-DBCO (68) in example 13.

FIG. 21 schematically illustrates a reaction scheme of preparing 30kmPEG-Lys(PEG₂-Mal)-2(Val-Cit-PAB-DEA-SN38) (69) in example 14.

FIG. 22 schematically illustrates a reaction scheme of preparing 30kmPEG-Lys(PEG₂-Mal)-3(Val-Cit-PAB-DEA-SN38) (70) in example 15.

FIG. 23 schematically illustrates a reaction scheme of preparing 30kmPEG-Lys(PEG₂-Mal)-4(Val-Cit-PAB-DEA-SN38) (71) in example 16.

FIG. 24 schematically illustrates a reaction scheme of preparing 20kmPEG-Lys(PEG₂-Mal)-2(Val-Cit-PAB-DEA-SN38) (73) in example 17.

FIG. 25 schematically illustrates a reaction scheme of preparing Mal-PEG₂-20kPEG-2(Val-Cit-PAB-DEA-SN38) (76) in example 18.

FIG. 26 schematically illustrates a reaction scheme of preparing Val-Cit-PAB-DEA-Dxd (81) in example 19.

FIG. 27 schematically illustrates a reaction scheme of preparing 20kmPEG-Lys(PEG₂-Mal)-2(Val-Cit-PAB-DEA-Dxd) (83) in example 20.

FIG. 28 schematically illustrates a reaction scheme of preparing of 30kmPEG(SCAHer2xSCAHer2)-2(Val-Cit-PAB-DEA-SN38) (86) in example 23.

FIG. 29 schematically illustrates a reaction scheme of preparing of 30kmPEG(SCAPDL1xSCACD47)-2(Val-Cit-PAB-DEA-SN38) (87) in example 24.

FIG. 30 schematically illustrates a reaction scheme of preparing of 30kmPEG(SCAHer2xSCAHer2)-3(Val-Cit-PAB-DEA-SN38) (88) in example 25.

FIG. 31 schematically illustrates a reaction scheme of preparing of 30kmPEG(SCAPDL1xSCACD47)-3(Val-Cit-PAB-DEA-SN38) (89) in example 26.

FIG. 32 schematically illustrates a reaction scheme of preparing of 30kmPEG(SCAPDL1xSCACD47)-4(Val-Cit-PAB-DEA-SN38) (90) in example 27.

FIG. 33 schematically illustrates a reaction scheme of preparing of 20kmPEG(SCAPDL1xSCACD47)-2(Val-Cit-PAB-DEA-SN38) (91) in example 28.

FIG. 34 schematically illustrates a reaction scheme of preparing of SCAPDL1xSCACD47-20kPEG-2(Val-Cit-PAB-DEA-SN38) (92) in example 29.

FIG. 35 schematically illustrates a reaction scheme of preparing of 30kmPEG(SCAPDL1xSCACD47)-2(Val-Cit-PAB-DEA-Dxd) (93) in example 30.

FIG. 36 schematically illustrates a reaction scheme of preparing of 30kmPEG(SCAc-MetxSCAc-Met)-2(Val-Cit-PAB-DEA-SN38) (94) in example 31.

FIG. 37 illustrates in vitro cytotoxicity of compound 86 and compound 88 to tumor cell line in example 32.

FIG. 38 illustrates in vitro cytotoxicity of compound 87, 89, 90, 91 and 92 to tumor cell line in example 33.

FIG. 39 illustrates in vitro cytotoxicity of compound 93 to tumor cell line in example 34.

FIG. 40 illustrates in vitro cytotoxicity of compound 94 to tumor cell line in example 35.

### DETAILED DESCRIPTION OF THE INVENTION

In this invention, a PEGylated mono- or multi-specific antibody hydroxyl-bearing drug conjugates, of which the hydroxyl group of a payload is reacted to link the payload to an antibody, are provided. With this invention, it is possible to produce an ADC with hydroxyl bearing cytotoxic payloads that are stable during blood circulation until the target is reached so that the payloads could be internalized and released inside of target cells to kill target cells.

Furthermore, this invention provides a novel antibody structure format of PEGylated mono- or bispecific single chain antibody hydroxyl-bearing drug conjugate that not only shows no toxicity mediated by Fc component of IgG based antibodies to megakaryocytes or other normal cells and increases therapeutic window, but also enhances the anti-tumor effect of the conjugate with increased tumor penetration, internalization and lysosome trafficking. Accordingly, this invention expands current ADC technologies to allow the vast number of cytotoxic hydroxyl-bearing compounds to be used as ADC payloads and improves current cancer therapy for the treatment of solid tumors.

### IV. Conjugate

In one aspect of the invention, compounds of formula (Ia) are provided:

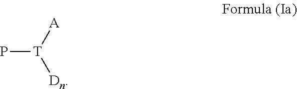

In the compound, P can be a non-immunogenic polymer. T can be a multi-functional moiety, such as a trifunctional small molecule linker moiety and have at least one functional group that is capable of site-specific conjugation with an antibody or protein. A can be any mono-specific or multi-specific antibody or protein, such as a full length antibody, a single chain antibody, a nanobody or any antigen binding fragment thereof, or a combination thereof D can be any hydroxy-bearing cytotoxic small molecule or peptide (n=1 to 25), and each D can be the same or different.

In particular, an aspect of the invention provides a conjugate of Formula Ib or Ic:

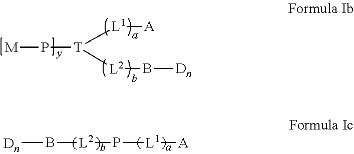

In the conjugate of Formula Ib or Formula Ic, P can be a non-immunogenic polymer such as a PEG;
M can be H or a terminal capping group selected from C₁₋₅₀ alkyl and aryl, wherein one or more carbons of said alkyl are optionally replaced with a heteroatom;
y can be an integer selected from 1, 2, 3, 4, 5, 6, 7, 8, 9, 10;
T can be a moiety having two or more functional groups, wherein the linkage between T and (L¹)ₐ and the linkage between T and (L²)_{b} can be the same or different;
Each of L¹ and L² can be independently a bifunctional linker;
Each of a and b can be an integer selected from 0, 1, 2, 3, 4, 5, 6, 7, 8, 9, 10;
B can be a branched linker, wherein each branch can comprise an extension spacer (optional), a trigger unit, one or more self-immolating spacer or any combination thereof, wherein the trigger unit can be an amino acid sequence or a β-glucoronide or β-galactoside trigger moiety cleavable by an enzyme such as cathepsin B, plasmin, matrix metalloproteinases (MMPs), β-glucuronidases, or β-galactosidases; a pH liable linker that can trigger the release of hydroxyl-bearing drug D or its derivatives at acidic pH conditions, or a disulfide bond linker that can trigger the release of hydroxyl-bearing drug D or its derivatives by glutathione, thioredoxin family members (WCGH/PCK) or thio reductase.

A can be any mono-specific or multi-specific antibody or antigen binding protein including an antibody fragment, a single chain antibody, a nanobody (a single-domain antibody) or any antigen binding fragment, which is monovalent or multivalent for the antigens;
D can be any hydroxyl-bearing cytotoxic small molecule or peptide or derivative thereof and can be released from B through either enzymatic cleavage and/or self-immolating mechanism or pH induced hydrolysis; each hydroxy-bearing D can be the same or different;
n can be an integer selected from 1, 2, 3, 4, 5, 6, 7, 8, 9, 10, 11, 12, 13, 14, 15, 16, 17, 18, 19, 20, 21, 22, 23, 24 and 25.

In some embodiments, each branch of B comprises a trigger moiety, e.g. an amino acid sequence or a disulfide moiety or a β-glucoronide or β-galactoside, connected to the hydroxyl-bearing drug D via one or more self-immolating spacer. Examples of self-immolating spacers include but not limit to the following:

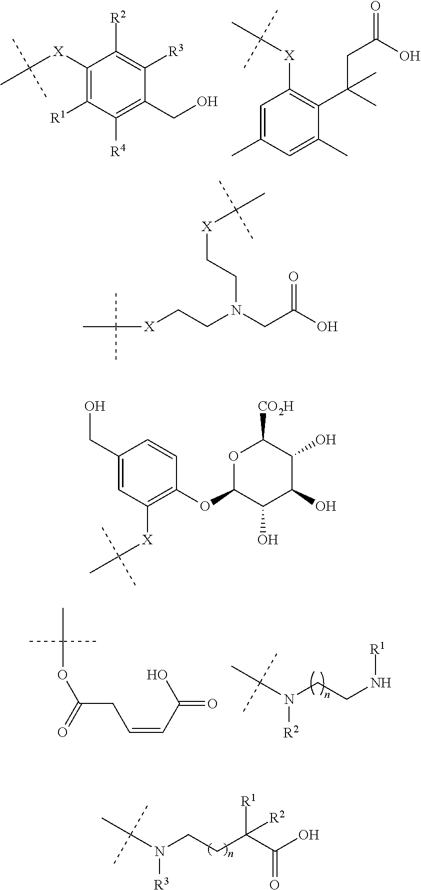

wherein X=0 or NH or S, n=1 or 2, R¹, R², R³, R⁴ can be H, C₁₋₁₀ alkyl or —(CH₂CH₂O)ₘ—CH₃, wherein m is an integer from 1 to 10.

In some embodiments, each branch of B can comprise a disulfide bond linker that can trigger the release of hydroxyl-bearing drug D or its derivatives at tumor site and/or inside of the tumor cell by enzymatic cleavage, e.g. by glutathione, thioredoxin family members (WCGH/PCK) or thio reductase, followed by one or more self-immolating mechanism.

In some embodiments, A is a single chain bispecific antibody that is able to bind to two different antigens such as PDL1 and CD47 (SCAPDL1xSCACD47).

In some embodiments, amino acid sequence of SCAPDL1xSCACD47could be:

(SEQ ID NO. 1)
DIVMTQSPLSLPVTPGEPASISCRSSQSIVYSNGNTYLGWYLQKP
GQSPQLLIYKVSNRFSGVPDRFSGSGSGTDFTLKISRVEAEDVGV
YYCFQGSHVPYTFGQGTKLEIKGGSGGSGGSGGSGGQVQLVQSGA
EVKKPGASVKVSCKASGYTFTNYNMHWVRQAPGQRLEWMGTIYPG
NDDTSYNQKFKDRVTITADTSASTAYMELSSLRSEDTAVYYCARG
GYRAMDYWGQGTLVTVSSGCGGSSGGSDIQMTQSPSSLSASVGDR
VTITCRASQDVSTAVAWYQQKPGKAPKLLIYSASFLYSGVPSRFS
GSGSGTDFTLTISSLQPEDFATYYCQQYLYHPATFGQGTKVEIKG
GGGSGGGGSGGGGSEVQLVESGGGLVQPGGSLRLSCAASGFTFSD
SWIHWVRQAPGKGLEWVAWISPYGGSTYYADSVKGRFTISADTSK
NTAYLQMNSLRAEDTAVYYCARRHWPGGFDYWGQGTLVTHHHHHH.

In some embodiments, A is a single chain bispecific antibody that is able to bind to two different epitopes on two Her2 antigens such as SCAHer2(1)xSCAHer2(2).

In some embodiments, amino acid sequence of SCAHer2(1)xSCAHer2(2) could be:

(SEQ ID NO. 2)
DIQMTQSPSSLSASVGDRVTITCKASQDVSIGVAWYQQKPGKAPK
LLIYSASYRYTGVPSRFSGSGSGTDFTLTISSLQPEDFATYYCQQ
YYIYPYTFGQGTKVEIKRTGGSGGSGGSGGSGGEVQLVESGGGLV
QPGGSLRLSCAASGFTFTDYTMDWVRQAPGKGLEWVADVNPNSGG
SIYNQRFKGRFTLSVDRSKNTLYLQMNSLRAEDTAVYYCARNLGP
SFYFDYWGQGTLVTVSSGCGSGGSGGSGGSGGEVQLVESGGGLVQ
PGGSLRLSCAASGFNIKDTYIHWVRQAPGKGLEWVARIYPTNGYT
RYADSVKGRFTISADTSKNTAYLQMNSLRAEDTAVYYCSRWGGDG
FYAMDYWGQGTLVTVSSGGSGGSGGSGGSGGDIQMTQSPSSLSAS
VGDRVTITCRASQDVNTAVAWYQQKPGKAPKLLIYSASFLYSGVP
SRFSGSRSGTDFTLTISSLQPEDFATYYCQQHYTTPPTFGQGTKV
EIKRTHHHHHH

In some embodiments, A is a single chain mono-specific antibody that is able to bind to two same epitopes on two Her2 antigens such as SCAHer2(1)xSCAHer2(1).

In some embodiments, amino acid sequence of SCAHer2(1)xSCAHer2(1) could be:

(SEQ ID No. 3)
DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIKRTGGSGGSGGSGGSGGEVQLVESGGGLV
QPGGSLRLSCAASGFNIKDTYIHWVRQAPGKGLEWVARIYPTNGY
TRYADSVKGRFTISADTSKNTAYLQMNSLRAEDTAVYYCSRWGGD
GFYAMDYWGQGTLVTVSSGCGSGGSGGSGGSGGDIQMTQSPSSLS
ASVGDRVTITCRASQDVNTAVAWYQQKPGKAPKLLIYSASFLYSG
VPSRFSGSRSGTDFTLTISSLQPEDFATYYCQQHYTTPPTFGQGT
KVEIKRTGGSGGSGGSGGSGGEVQLVESGGGLVQPGGSLRLSCAA
SGFNIKDTYIHWVRQAPGKGLEWVARIYPTNGYTRYADSVKGRFT
ISADTSKNTAYLQMNSLRAEDTAVYYCSRWGGDGFYAMDYWGQGT
LVTVSSHHHHHH

In some embodiments, A is a single chain bispecific antibody that is able to bind to two different antigens such as Her2 and Her3 (SCAHer2xSCAHer3).

In some embodiments, amino acid sequence of SCAHer2IVxSCAHer3 could be:

(SEQ ID NO. 4)
DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIKRTGGSGGSGGSGGSGGEVQLVESGGGLV
QPGGSLRLSCAASGFNIKDTYIHWVRQAPGKGLEWVARIYPTNGY
TRYADSVKGRFTISADTSKNTAYLQMNSLRAEDTAVYYCSRWGGD
GFYAMDYWGQGTLVTVSSGCGSGGSGGSGGSGGQVQLQQWGAGLL
KPSETLSLTCAVYGGSFSGYYWSWIRQPPGKGLEWIGEINHSGST
NTNPSLKSRVTISVETSKNQFSLKLSSVTAADTAVYYCARDKWTW
YFDLWGRGTLVTVSSGGSGGSGGSGGSGGDIEMTQSPDSLAVSLG
ERATINCRSSQSVLYSSSNRNYLAWYQQNPGQPPKLLIYWASTRE
SGVPDRFSGSGSGTDFTLTISSLQAEDVAVYYCQQYYSTPRTFGQ
GTKVEIKHHHHHH.

In some embodiments, A is a single chain bispecific antibody that is able to bind to two different antigens such as Met1 and Met2 (SCAc-MetlxSCAc-Met2).

In some embodiments, amino acid sequence of SCAc-MetlxSCAc-Met2 could be:

(SEQ ID NO. 5)
DIQMTQSPSSLSASVGDRVTITCSVSSSVSSIYLHWYQQKPGKAP
KLLIYSTSNLASGVPSRFSGSGSGTDFTLTISSLQPEDFATYYCI
QYSGYPLTFGGGTKVEIKGGSGGSGGSGGSGGQVQLVQSGAEVKK
PGASVKVSCKASGYTFTDYYMHWVRQAPGQGLEWMGRVNPNRGGT
TYNQKFEGRVTMTTDTSTSTAYMELRSLRSDDTAVYYCARTNWLD
YWGQGTTVTVSGCGSGGSGGSGGSGGQVQLVQSGAEVKKPGASVK
VSCKASGYIFTAYTMHWVRQAPGQGLEWMGWIKPNNGLANYAQKF
QGRVTMTRDTSISTAYMELSRLRSDDTAVYYCARSEITTEFDYWG
QGTLVTVSSGGSGGSGGSGGSGGDIVMTQSPDSLAVSLGERATIN
CKSSESVDSYANSFLHWYQQKPGQPPKLLIYRASTRESGVPDRFS
GSGSGTDFTLTISSLQAEDVAVYYCQQSKEDPLTFGGGTKVEIKR
HHHHHH.

In some embodiments, amino acid sequence of SCAc-Met(1)xSCAc-Met(2) could be:

(SEQ ID No. 6)
DIMMSQSPSSLTVSVGEKVTVSCKSSQSLLYTSSQKNYLAWYQQK
PGQSPKLLIYWASTRESGVPDRFTGSGSGTDFTLTITSVKADDLA
VYYCQQYYAYPWTFGGGTKLEIKGGSGGSGGSGGSGGQVQLQQSG
PELVRPGASVKMSCRASGYTFTSYWLHWVKQRPGQGLEWIGMIDP
SNSDTRFNPNFKDKATLNVDRSSNTAYMLLSSLTSADSAVYYCAT
YGSYVSPLDYWGQGTSVTVSSGCGSGGSGGSGGSGGQVQLVESGP
GLVKPSQTLSLTCAVSGGSITTNYYYWSWIRQSPGKGLEWMGVIA
YDGSTDYSPSLKSRTSISRDTSKNQFSLQLSSVTPEDTAVYYCAR
DVRVIATGWATANALDAWGQGTLVTVSSGGSGGSGGSGGSGGQSV
LTQPPSVSGSPGKTVTISCAGTSSDVGYGNYVSWYQQLPGTAPKL
LIFAVSYRASGIPDRFSGSKSGNTAFLTISGLQSEDEADYYCASY
RSSNNAAVFGGGTHLTVLHHHHHH.

In some embodiments, hydroxyl-bearing drug D can be released either at tumor site or inside of tumor cells by either enzymatic trigger or pH induced hydrolysis followed by one or more self-immolating mechanism.

In some embodiments, hydroxyl-bearing drug D can be selected from any DNA crosslinker agent, microtubule inhibitor, DNA alkylator, topoisomerase inhibitor, protein degrader, STING agonist or a combination thereof.

In some embodiments, hydroxyl-bearing drug D can be selected from Dxd, SN38, calicheamicins, pyrrolobenzodiazepines, sibiromycin, tomaymycin, duocarmycins, neothramycins, DC-81, psymberin, vinca alkaloid, laulimalide, taxane, tubulysins, rhizoxin, discodermolide, taccalonolide A or B or AF or AJ, taccalonolide AI-epoxide, epothilone A and B, paclitaxel, docetaxel, doxorubicin, camptothecin, tafuramycin A, PNU-159682, uncialamycin, β-amanitin, amatoxins, thailanstatins or any hydroxyl-bearing cytotoxic compounds or their analogs/derivates thereof, or a combination thereof.

In some embodiments, D is SN38 or Dxd (a potent topoisomerase I inhibitor) or duocarmycin (a DNA alkylator) or their analogs/derivatives, or a combination thereof.

In a further embodiments, hydroxyl-bearing drug D is linked to a double-self-immolating spacer such as ethylenediamine (EDA) or its derivatives, and 4-aminobenzyl alcohol (PAB), which in turn is linked to a trigger moiety such as valine-citrulline Val-Cit-PAB-EDA-D.

In one aspect of this invention, methods for preparing PEGylated hydroxyl-bearing drug conjugate that is capable of site-specific conjugating to a protein or antibody, such as antibody fragment or single chain mono- or multi-specific antibody, are provided. In another aspect of this invention, methods for preparing PEGylated single chain bispecific antibody hydroxyl-bearing drug conjugate are provided.

To synthesize PEGylated single-chain bispecific antibody hydroxyl-bearing drug conjugate, coding sequence or a vector carrying a coding sequence of mono-specific single-chain antibody with valence of 1 to 5 or single-chain bispecific antibody can be synthesized and introduced into, e.g., the CHO expression systems. The proteins can be expressed and purified as described previously (WO2018075308).

For the synthesis of PEGylated hydroxyl-bearing drug conjugate with a side chain that has a site-specific conjugation functional group, a terminal functional group of PEG such as hydroxyl or carboxyl group and the like, can be activated and conjugated with a trifunctional small molecule moiety such as Boc or Fmoc protected lysine to form a terminal branched heterobifunctional PEG followed by removal of protection group. The PEGylated compound after deprotection can be coupled with a small molecule linker that has site-specific conjugation functional group such as maleimide or DBCO to form PEG-Lys(Mal)-OH or PEG-Lys(DBCO)-OH. PEG-Lys(Mal)-OH or PEG-Lys(DBCO)-OH can then be coupled with a branch moiety, of which each branch is linked with a hydroxyl-bearing drug D, e.g. SN38, via a trigger unit and a double-self-immolating spacer to form a PEGylated hydroxyl-bearing drug conjugate such as PEG-lys(Mal)-B(Val-Cit-PAB-EDA-SN38)ₙ or PEG-lys(DBCO)-B(Val-Cit-PAB-EDA-SN38)ₙ, wherein n is an integer from 1 to 20, e.g. 4. The final step of synthesis is site-specific conjugation of PEGylated hydroxyl-bearing drug conjugate to a thiol or azide tagged single chain mono-specific or bispecific antibody to form the compound of Formula Ia. and Ib. Alternatively, PEGylated hydroxyl-bearing drug conjugate Mal-PEG-B-(Val-Cit-PAB-EDA-SN38)ₙ or DBCO-PEG-B-(Val-Cit-PAB-EDA-SN38)ₙ, wherein n is an integer e.g. 4, can be synthesized from commercial available heterobifunctional PEG using similar procedures and site-specific conjugation of PEGylated hydroxyl-bearing drug conjugate with a thiol or azide tagged single chain bispecific antibody forms the compound of Formula Ic.

### II. Polyethylene Glycol (PEG) Moiety

In one embodiment of the present invention, the linear PEG can be of the formula:

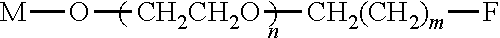

In the formula, n can be an integer from 1 to about 2300 to preferably provide a polymer having a total molecule weight of from 3000 to 100000 Dalton or greater if desired. M can be H, methyl or other low molecule weight alkyl. Non-limiting examples of M include H, methyl, ethyl, isopropyl, propyl, butyl or F₁(CH₂)_{q}CH₂, wherein F and F₁ can be independently a terminal functional group such as hydroxyl, carboxyl, thiol, halide, amino group and the like, which is capable of being functionalized, activated and/or conjugated to a small molecule spacer or linker. Q and m can be any integer from 0 to 10.

In another embodiment of present invention, the method can also be carried out with an alternative branched PEG. The branched PEG can be of the formula:

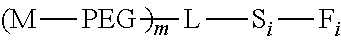

In this formula, PEG is polyethylene glycol. M can be an integer between 2 to 10 to preferably provide a branched PEG having a total molecule weight of from 3000 to 100000 Dalton or greater if desired. M can be methyl or other low molecule weight alkyl. L can be a functional linkage moiety to that two or more PEGs are attached. Non-limiting examples of such linkage moiety are: any amino acids such as glycine, alanine, lysine, or 1,3-diamino-2-propanol, triethanolamine, any 5 or 6 member aromatic or aliphatic rings with more than two functional groups attached. S is any non-cleavable spacer. F can be a terminal functional group such as hydroxyl, carboxyl, thiol, amino group. I is 0 or 1. When i equals to 0, the formula is shown as:

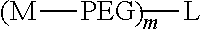

wherein: each variables of PEG, m, M or L have the same definitions as above.

The method of the present invention can also be carried out with alternative polymeric substances such as dextrans, carbohydrate polymers, polyalkylene oxide, polyvinyl alcohols or other similar non-immunogenic polymers, the terminal groups of which are capable of being functionalized or activated. The foregoing list is merely illustrative and not intended to restrict the type of non-antigenic polymer suitable for use herein.

### III. Trifunctional Linker T

T represents a trifunctional linker, connecting with P, (L¹)ₐ and (L²)_{b}. T can be derived from molecules with any combination of three functional groups, non-limiting examples of which include hydroxyl, amino, hydrazinyl, azide, alkene, alkyne, carboxyl (aldehyde, ketone, ester, carboxylic acid, anhydride, acyl halide), thiol, disulfide, nitrile, epoxide, imine, nitro and halide. The functional groups in a trifunctional linker may be the same or different. In some embodiments, one or two of the functional groups may be protected to achieve selective conjugation with other reaction partners. A variety of protecting groups are known in the art, including for example, those shown in Advanced Organic Chemistry by March (Third Edition, 1985, Wiley and Sons, New York). A functional group may also be converted into other groups before or after the reaction between T and another reaction partner. For example, a hydroxyl group may be converted into a mesylate or a tosylate group. A halide may be replaced with an azido group. An acid functional group of T may be converted to an alkyne function group by coupling with an amino group bearing a terminal alkyne.

In exemplary embodiments, T is derived from 1,3-diamino-2-propanol, triethanolamine, lysine, aspartic acid, glutamic acid, serine or tyrosine. One or more of the functional groups on these molecules may be protected for selective reactions. In some embodiments, T is derived from a Boc-protected lysine.

IV. Bifunctional Linker L¹ and L²

Both linkers L¹ and L² comprise linker chains that may be independently selected from
(CH₂)ₐXY(CH₂)_{b}—,
—X(CH₂)ₐO(CH₂CH₂O)_{c}(CH₂)_{b}Y—,
—(CH₂)ₐ-heterocyclyl-,
—(CH₂)ₐX—,
—X(CH₂)ₐY—,
—W₁—(CH₂)ₐC(O)NR₁(CH₂)_{b}O(CH₂CH₂O)_{c}(CH₂)_{d}X—,
—X(CH₂)ₐO(CH₂CH₂O)_{b}(CH₂)_{c}W₂C(O)(CH₂)_{d}Y—,
—W₃—(CH₂)ₐC(O)NR₁(CH₂)_{b}O(CH₂CH₂O)_{c}(CH₂)_{d}W₂C(O)(CH₂)ₑX—,
wherein a, b, c, d and e are each an integer independently selected from 0 to 25, e.g. 0-20, 0-15, 0-10, 0-5, 5-25, 5-20, 5-15, 5-10, 10-25, 10-20, 10-15, 15-25, 15-20 or 20-25, e.g. 0, 1, 2, 3, 4, 5, 6, 7, 8, 9, 10, 11, 12, 13, 14, 15, 16, 17, 18, 19, 20, 21, 22, 23, 24, 25; each of X and Y is independently selected from C(═O), NR₂, S, O, CR₃R₄ or Null; R₁, R₂, R₃ and R₄ independently represent hydrogen, C1-10 alkyl or (CH₂)₁₋₁₀C(═O); W₁ and/or W₃ is derived from a maleimido-based moiety and W₂ represents a triazolyl or a tetrazolyl containing group; the heterocyclyl group is selected from a maleimido-derived moiety or a tetrazolyl-based or a triazolyl-based moiety. Non-limiting examples of a maleimido-based moiety include N-succinimidyl 4-(maleimidomethyl)cyclohexanecarboxylate (SMCC), N-succinimidyl-4-(N-maleimidomethyl)-cyclohexane-1-carboxy-(6-amidocaproate) (LC-SMCC), κ-maleimidoundecanoic acid N-succinimidyl ester (KMUA), γ-maleimidobutyric acid N-succinimidyl ester (GMBS), ε-maleimidcaproic acid N-hydroxysuccinimide ester (EMCS), m-maleimidobenzoyl-N-hydroxysuccinimide ester(MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI). Alternatively, the heterocyclyl linkage group of the linker may be tetrazolyl or triazolyl which are formed by conjugations of different linker moieties such as DBCO and azide.

In some exemplary embodiments, (L¹)^{a} and (L²)_{b} can be selected from:

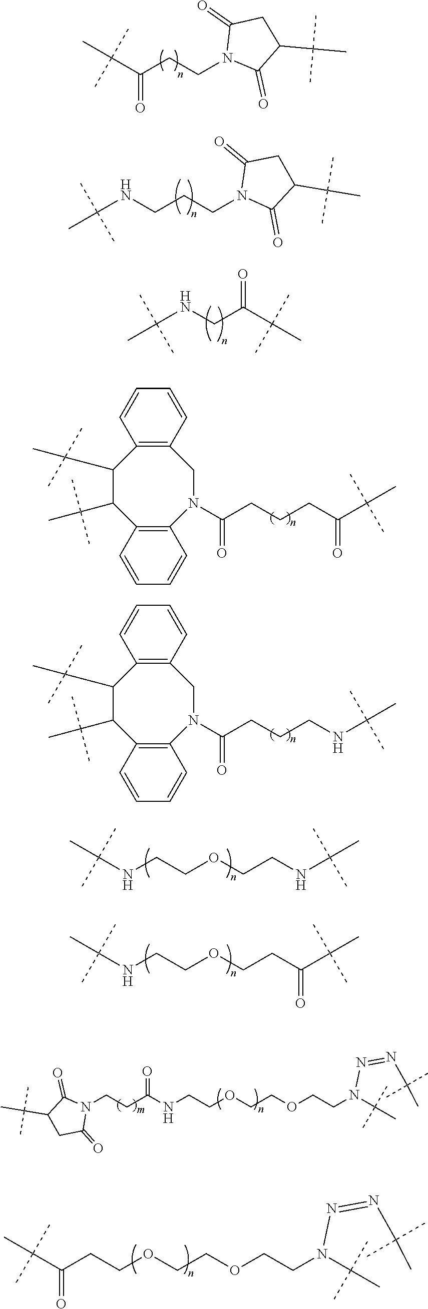

wherein n and m are integer and independently selected from 0 to 20.

In some other non-limiting exemplary embodiments, each linker unit can also be derived from a haloacetyl-based moiety selected from N-succinimidyl-4-(iodoacetyl)-aminobenzoate (SIAB), N-succinimidyl iodoacetate (SIA), N-succinimidyl bromoacetate (SBA), or N-succinimidyl 3-(bromoacetamido)propionate (SBAP).

### V. Branched Linker B

The branched linker B can comprise a branching unite, an extension spacer (optional), a trigger unit, one or more self-immolating spacer or any combination of such.

In some embodiments, a branching unite comprises structures that may be independently selected from:

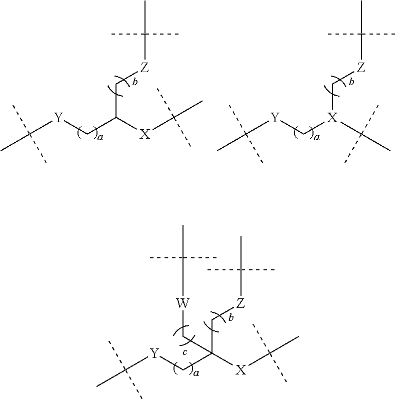

1. X, Y, Z or W=NR¹, NR², C(═O), O, N or null, wherein R¹ and R² independently represent hydrogen or C₁₋₁₀ alkyl group
2. a, b, c is integer from 0-10

In other embodiments, a branching unite comprises structures that may be independently selected from:

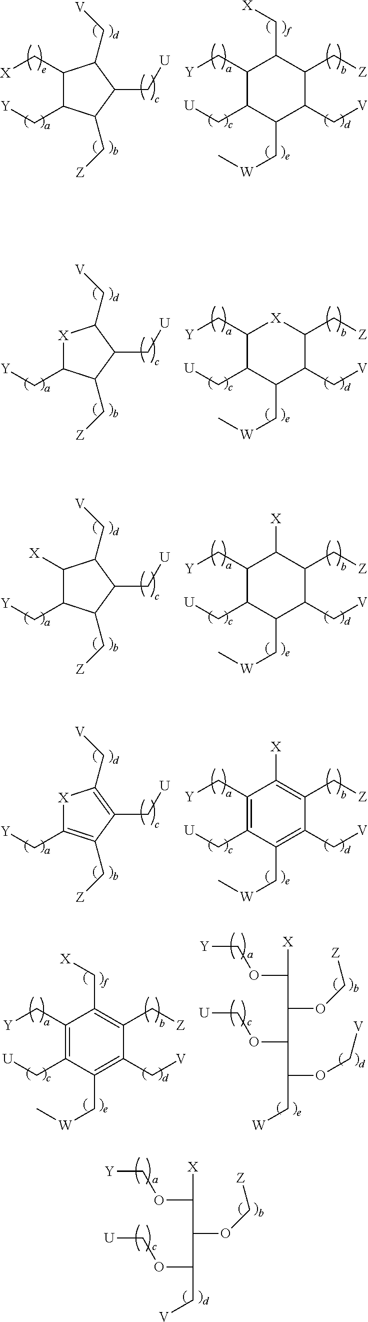

1. X, Y, Z, U, V, W=C(O), NR¹, NR², O, N or Null, wherein R¹ and R² independently represent hydrogen or C1-10 alkyl group
2. a, b, e, d, e=010

In some embodiments, an extension spacer in each branch comprises linker chains that may be independently selected from:
—X(CH₂)ₐO(CH₂CH₂O)_{b}(CH₂)_{c}Y—, —X(CH₂)ₐY—,
   or any combination thereof, wherein a, b, and c are each an integer selected from 0 to 25, all subunits included; X and Y may be selected independently from NR¹, NR², C(O), O, or Null, wherein R¹ and R²independently represent hydrogen or C₁₋₁₀ alkyl group.

In some embodiments, a branching unit (e.g. with two branches) with or without extension spacers can be joined by two or more branching units (e.g. with two branches) to form a branching unit with four branches.

In other embodiments, a trigger unit comprises any amino acid sequence or any carbohydrate moiety or a disulfide bond or a PH liable bond or any cleavable bond that can be enzymatically or chemically cleaved.

In some embodiments, a self-immolating spacer comprises structures that may be selected from:

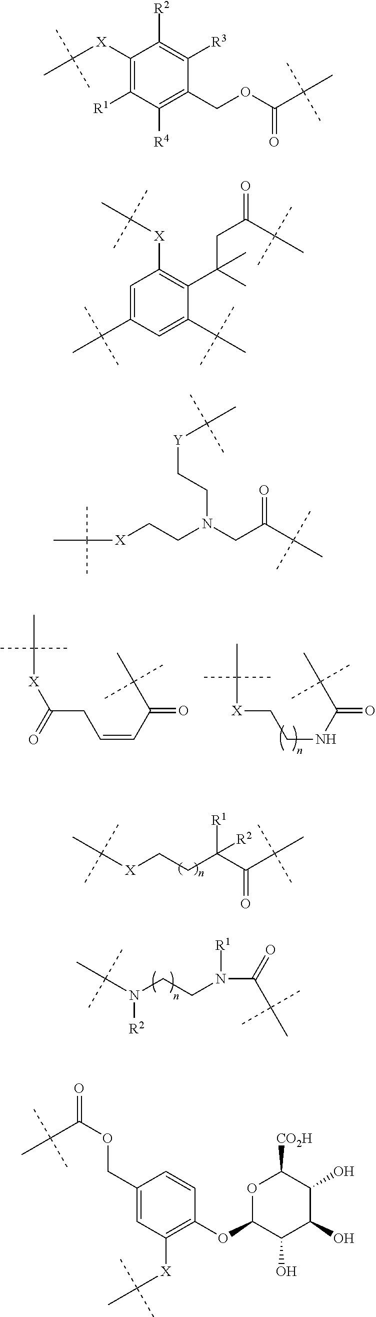

wherein n is 1 or 2; R¹, R², R³ and R⁴ independently represent hydrogen, C₁₋₁₀ alkyl or —(CH₂CH₂O)ₘCH₃, wherein m=1-10; X and Y can be NH or O or S.

In some embodiment, two self-immolating spacers can be connected to each other, e.g.

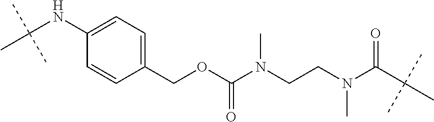

In some embodiments, the branched linker B can be selected from:

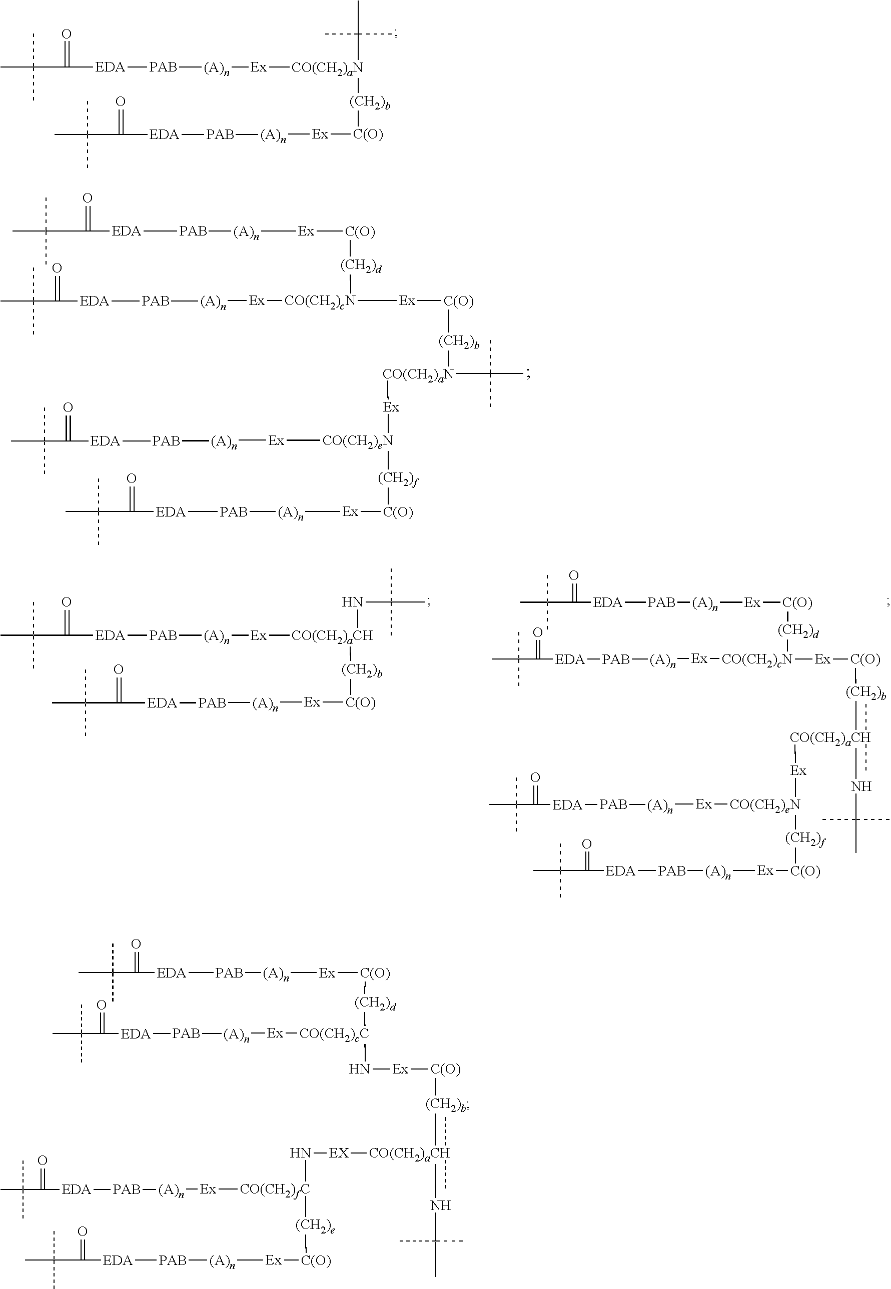

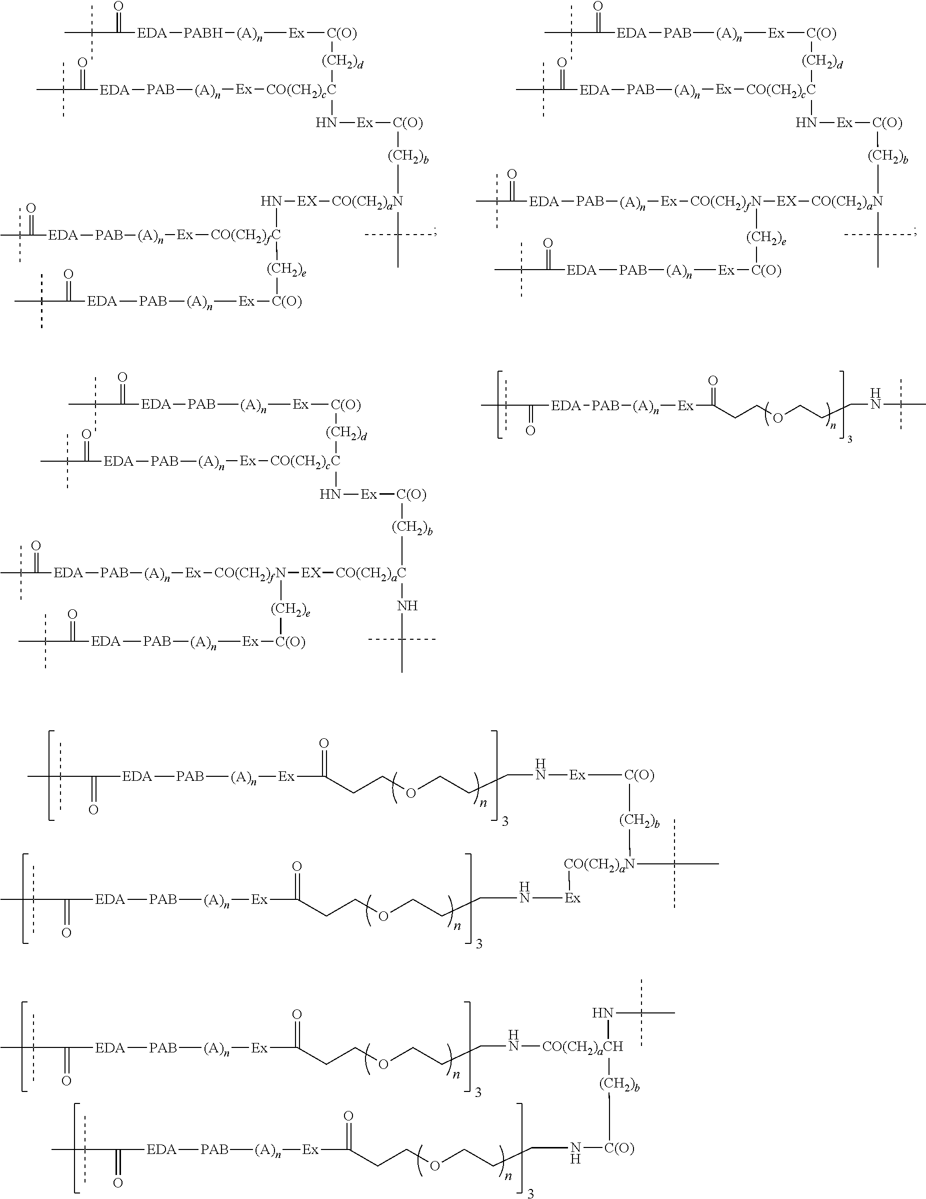

Wherein:
each of a, b, c, d, e and f is independently an integer selected from 1-25;
n is an integer selected from 1 to 10;
(A)ₙ is a trigger unit of amino acid sequence, each Ais an independent amino acid and n is any integer from 1-25;
PAB is 4-aminobenzyl alcohol;
EDA is HNR¹CH₂CH₂NHR² or HNR¹CH₂CH₂CH₂NHR², wherein R¹ and R² independently represent hydrogen, C₁₋₁₀ alkyl group or —(CH₂CH₂O)ₘCH₃, wherein m is any integer from 1-10;
Ex is an extension spacer that comprises linker chains that may be independently selected from:
—NR¹(CH₂)ₐO(CH₂CH₂O)_{b}(CH₂)_{c}C(O)—,
—C(O)(CH₂)ₐNR¹—,
—NR¹(CH₂)ₐO(CH₂CH₂O)_{b}(CH₂)_{c}NR²,
—NR¹(CH₂)ₐNR²—,
—NR¹(CH₂)ₐO(CH₂CH₂O)_{b}(CH₂)_{c}O—,
—O(CH₂)ₐNR¹—,
—C(O)(CH₂)ₐO—,
—O(CH₂)ₐO(CH₂CH₂O)_{b}(CH₂)_{c}C(O)—,
—C(O)(CH₂)ₐO(CH₂CH₂O)_{b}(CH₂)_{c}C(O)—,
—C(O)(CH₂)ₐC(O)—,
or Null;
   wherein a, b, and c are each an integer selected from 0 to 25, all subunits included; and R¹ and R²independently represent hydrogen or C₁₋₁₀ alkyl group.

In some other embodiments, the trigger unit of the amino acid sequence can be Val-Cit, Val-Ala, Val-Lys, Phe-Lys, Phe-Cit, Phe-Arg, Phe-Ala, Ala-Lys, Leu-Cit, Ile-Cit, Trp-Cit, D-Phe-Phe-Lys, Phe-Phe-Lys, Gly-Phe-Lys, Gly-Phe-Leu-Gly, or Ala-Leu-Ala-Leu, Gly-Gly-Phe-Gly.

For preferred embodiments, the amino acid sequence can be Val-Cit, Phe-Lys, or Val-Lys.

In some exemplary embodiments, branched linker B can be selected from:

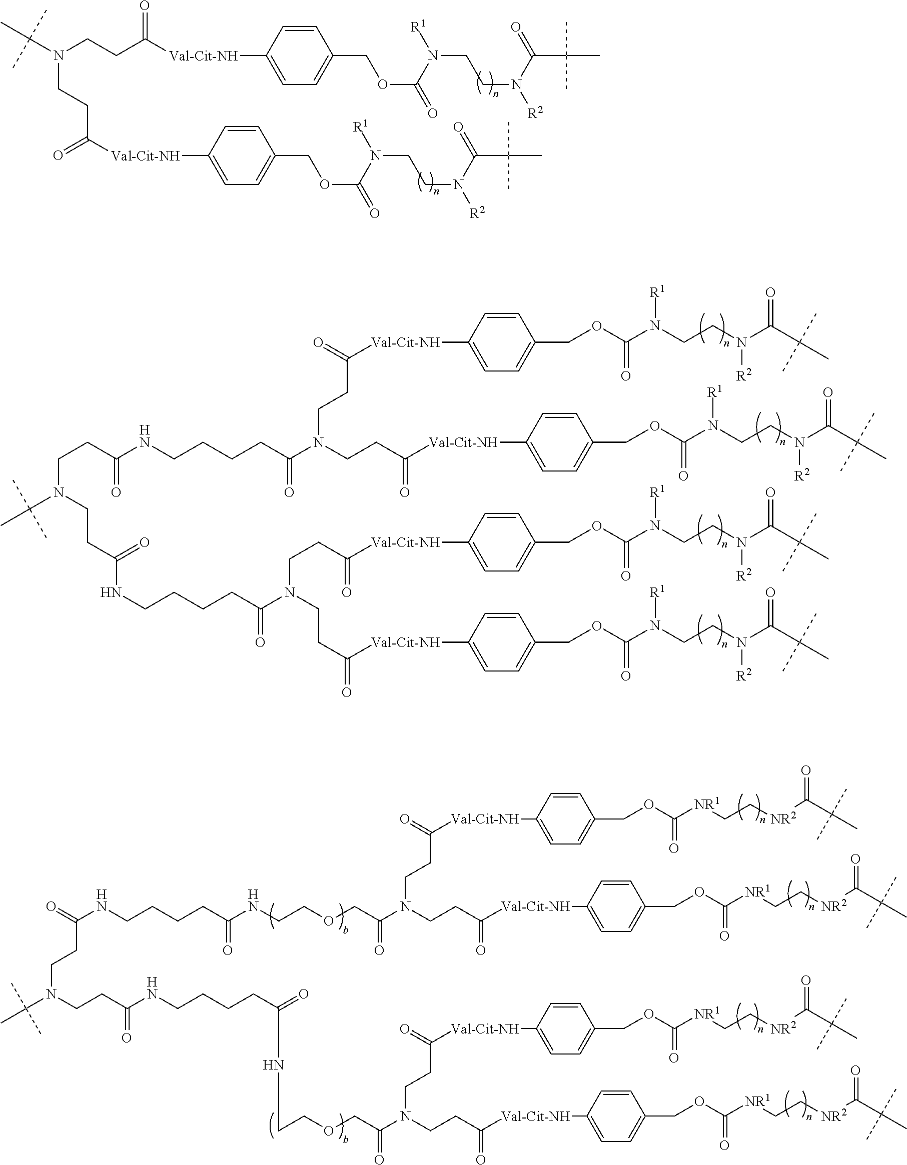

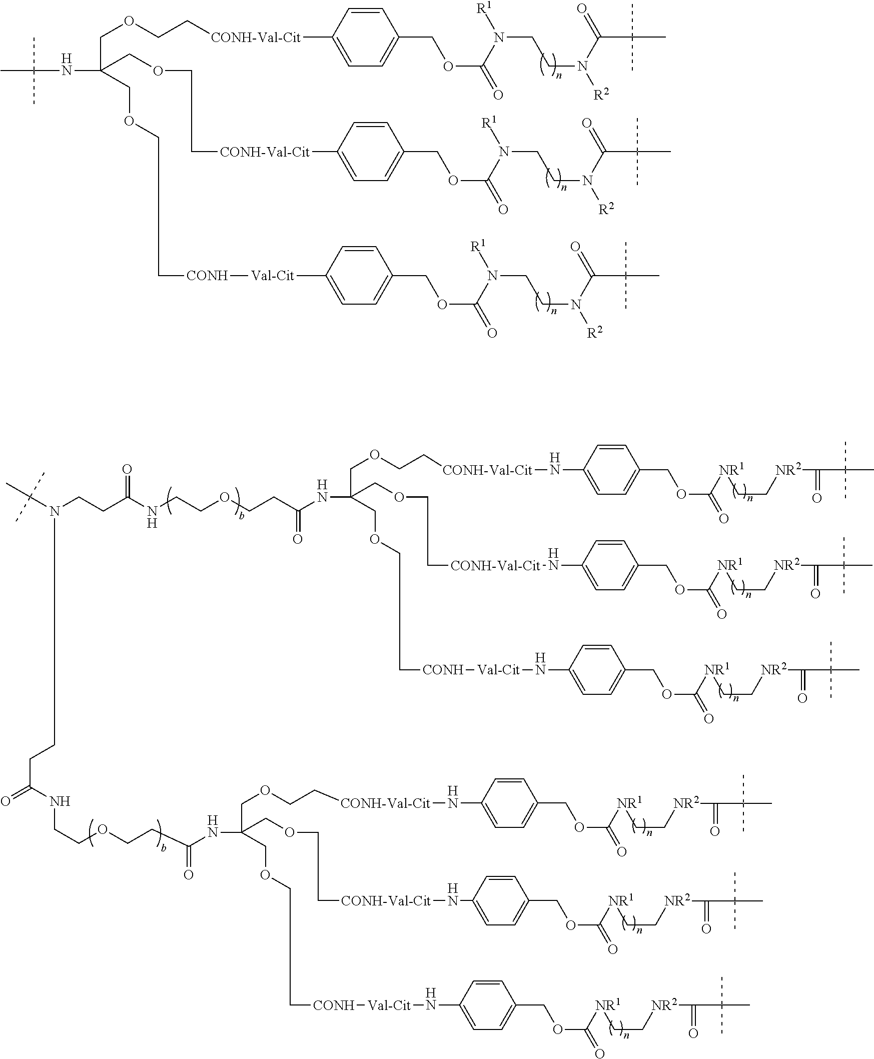

Wherein n=1 or 2; b is an integer selected from 1 to 10; each of R¹ and R² is independently selected from H, a low molecule weight alkyl or a low molecular weight PEG [—(CH₂CH₂O)ₘ—CH₃, wherein m=1-10].

### VL Linkage Group

Different moieties of the conjugates of the present invention can be connected via various chemical linkages. Examples include but are not limited to amide, ester, disulfide, ether, amino, carbamate, hydrazine, thioether, and carbonate. For instance, the terminal hydroxyl group of a PEG moiety (P) may be activated and then coupled with lysine (T) to provide a desirable linkage point between P and T of Formula Ia or Ib. The linkage group between T and (L¹)ₐ or between T and (L²)_{b} or between (L²)_{b} and B may be an amide resulting from the reaction between the amino group of a linker (L²)_{b} and the carboxyl group of Lysine (T) or between the carboxyl group of (L¹)ₐ and the amino group of T or between the carboxyl group of (L²)_{b} and the amino group of B. Depending on the desirable characteristics of the conjugate, suitable linkage groups may also be incorporated between the antibody moiety (A) and the adjacent linker L¹ or between any two amino acids or between an amino acid and para-aminobenzyl alcohol or between para-aminobenzyl alcohol and N,N′-Dimethylethylenediamine or its derivatives.

In some embodiments, the linkage group between different moieties of the conjugates may be derived from coupling of a pair of functional groups which bear inherent chemical affinity or selectivity for each other. These types of coupling or ring formation allow for site-specific conjugation for the introduction of a protein or antibody moiety to a PEGylated moiety. Non-limiting examples of these functional groups that lead to site-specific conjugation include thiol, maleimide, 2′-pyridyldithio variant, aromatic or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, and triarylphosphine, boronic acid, alkyne.

### VII. Cytotoxic Compound D

In some embodiments, D can be any hydroxyl-bearing compounds, include but not limit to vinca alkaloid, laulimalide, colchicine, tubulysins, cryptophycins, hemiasterlin, cemadotin, rhizoxin, discodermolide, taccalonolide A or B or AF or AJ, taccalonolide AI-epoxide, CA-4, epothilone A and B, taxane, paclitaxel, docetaxel, epothilone, iSGD-1882, centanamycin, PNU-159682, uncialamycin, indolinobenzodiazepine dimers, β-amanitin, amatoxins, thailanstatins, anthracycline, daunomycin, larotaxel, tesetaxel, ortataxel, CC-1065, Dxd, SN38, topotecan, CPT-11, camptothecin, rubitecan, bryostatin, callystatin, bizelesin, duocarmycin, eleutherobin, pancratistatin, sarcodictyin, spongistatin, estramustine, prednimustine, chlorozotocin, ranimustine, calicheamicin, dynemicin, esperamicin, neocarzinostatin chromophore, aclacinomysins, azithromycin, bleomycins, caminomycin, carzinophilin, chromomycins, daunorubicin, detorubicin, doxorubicin, epirubicin, esorubicin, idarubicin, marcellomycin, mycophenolic acid, nogalamycin, peplomycin, puromycin, quelamycin, rodorubicin, streptonigrin, streptozocin, tubercidin, ubenimex, zinostatin, fludarabine, ancitabine, azacytidine, 6-azauridine, carmofur, cytarabine (cytosine arabinoside, ara-C, gemcitabine, capecitabine, dideoxyuridine, doxifluridine, enocitabine, floxuridine, calusterone, epitiostanol, trilostane, elliptinium acetate, maytansinoids, ansamitocins, mitoxantrone, mopidamol, pentostatin, pirarubicin, etoposide, podophyllotoxin, rhizoxin, tenuazonic acid, T-2 mycotoxin, verracurin A, roridin A, anguidine, vindesine, mannomustine, mitobronitol, mitolactol, vinblastine, mitoxantrone, vincristine, vinorelbine, teniposide, xeloda, raloxifene, 4-hydroxytamoxifen, estradiol, trioxifene, keoxifene, LY117018, onapristone, bicalutamide, leuprolide, goserelin or its pharmaceutically acceptable salts, acids or derivatives thereof, or a combination thereof.

### VIII. Antibody and Target

A number of therapeutic antibodies against cell surface molecules and/or their ligands are known. These antibodies can be used for the selection and construction of tailor-made specific recognition binding moiety in the mono- or multi-specific PEGylated antibody hydroxyl-bearing drug conjugate. Examples include Monjuvi/Tafasitamab (CD19), Rituxan/MabThera/Rituximab (CD20), H7/Ocrelizumab (CD20), Zevalin/Ibrizumomab (CD20), Arzerra/Ofatumumab (CD20), HLL2/Epratuzumab, Inotuzomab (CD22), Zenapax/Daclizumab, Simulect/Basiliximab (CD25), Herceptin/Trastuzumab, Pertuzumab (Her2), Mylotarg/Gemtuzumab (CD33), Raptiva/Efalizumab (Cd11a), Erbitux/Cetuximab (EGFR, epidermal growth factor receptor), IMC-1121B (VEGF receptor 2), Tysabri/Natalizumab (α4-subunit of α4β1 and α4β7 integrins), ReoPro/Abciximab (gpIIb-gpIIa and αvβ3-integrin), Orthoclone OKT3/Muromonab-CD3 (CD3), Benlysta/Belimumab (BAFF), Tolerx/Oteliximab (CD3), Soliris/Eculizumab (C5 complement protein), Actemra/Tocilizumab (IL-6R), Panorex/Edrecolomab (EpCAM, epithelial cell adhesion molecule), CEA-CAM5/Labetuzumab (CD66/CEA, carcinoembryonic antigen), Tecentriq/atezolizumab (anti-PDL1), Imfinzi/durvalumab (anti-PDL1), CT-11 (PD-1, programmed death-1 T-cell inhibitory receptor, CD-d279), H224GII (c-Met receptor), SAR3419 (CD19), IMC-A12/Cixutumumab (IGF-1R, insulin-like growth factor 1 receptor), MEDI-575 (PDGF-R, platelet-derived growth factor receptor), CP-675, 206/Tremelimumab (cytotoxic T lymphocyte antigen 4), RO5323441 (placenta growth factor or PGF), HGS1012/Mapatumumab (TRAIL-R1), SGN-70 (CD70), Vedotin (SGN-35)/Brentuximab (CD30), and ARH460-16-2 (CD44).

A number of cell surface markers and their ligands are known. For example cancer cells have been reported to express at least one of the following cell surface markers and/or ligands, including but not limited to, carbonic anhydrase IX, α-fetoprotein, α-actinin-4, A3 (antigen specific for A33 antibody), ART-4, B7, Ba-733, BAGE, BrE3-antigen, CA125, CAMEL, CAP-1, CASP-8/m, CCCL19, CCCL21, CD1, CD1a, CD2, CD3, CD4, CDS, CD8, CD1-1A, CD14, CD15, CD16, CD18, CD19, CD20, CD21, CD22, CD23, CD25, CD29, CD30, CD32b, CD33, CD37, CD38, CD40, CD40L, CD45, CD46, CD47, CD54, CD55, CD59, CD64, CD66a-e, CD67, CD70, CD74, CD79a, CD80, CD83, CD95, CD126, CD133, CD138, CD147, CD154, CDC27, CDK-4/m, CDKN2A, CXCR4, CXCR7, CXCL12, HIF-1-α, programmed death-ligand 1/2 (PD-L1, PD-L2, or CD274 and CD273), colon-specific antigen-p (CSAp), CEA (CEACAM5), CEACAM6, c-met, DAM, EGFR, EGFRvIII, EGP-1, EGP-2, ELF2-M, Ep-CAM, Flt-1, Flt-3, folate receptor, G250 antigen, GAGE, GROB, HLA-DR, HM1.24, human chorionic gonadotropin (HCG) and its subunits, HER2/neu, HMGB-1, hypoxia inducible factor (HIF-1), HSP70-2M, HST-2 or 1a, IGF-1R, IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, insulin-like growth factor-1 (IGF-1), KC4-antigen, KS-1-antigen, KS 1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5, MUM-1/2, MUM-3, NCA66, NCA95, NCA90, pancreatic cancer mucin, placental growth factor, p53, PLAGL2, prostatic acid phosphatase, PSA, PRAME, PSMA, PiGF, ILGF, ILGF-1R, IL-6, IL-25, RS5, RANTES, T101, SAGE, 5100, survivin, survivin-2B, TAC, TAG-72, tenascin, TRAIL receptors, TNF-α, Tn-antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, c-Met, an oncogene marker and an oncogene product (Sensi, M. et al., Clin. Cancer Res., 2006, 12, 5023-5032; Parmiani, G. et al., J. Immunol., 2007, 178, 1975-1979; Castelli, C. et al., Cancer Immunol. Immunother., 2005, 54, 187-207). Thus, antibodies recognizing such specific cell surface receptors or their ligands can be used for specific and selective recognition binding moieties in the multi-specific ADC of this invention, targeting and binding to a number of cell surface markers or ligands that are associated with a disease. Antibodies against the above-mentioned antigens can be used as the binding domain or moieties to make mono- or multi-specific PEGylated antibody hydroxyl-bearing drug conjugate of this invention.

In some embodiments, for the treatment of cancer/tumors, mono- or multi-specific PEGylated antibody hydroxyl-bearing drug conjugates are used to target tumor-associated antigens (TAAs), reported in Herberman, “Immunodiagnosis of Cancer”, in Fleisher ed., “The Clinical Biochemistry of Cancer”, page 347 (American Association of Clinical Chemists, 1979) and in U.S. Pat. Nos. 4,150,149; 4,361,544; 4,444,744. Reports on tumor associated antigens can also be found in Mizukami, Y. et al. Nature Med., 2005, 11, 992-997; Hatfield, K. J. et al., Curr. Cancer Drug Tar., 2005, 5, 229-248; Vallbohmer, D. et al., J. Clin. Oncol. 2005, 23, 3536-3544; and Ren, Y. et al., Ann. Surg. 2005, 242, 55-63, each incorporated herein by reference with respect to the TAAs identified. Where the disease involves a lymphoma, leukemia or autoimmune disorder, targeted antigens may be selected from the group consisting of CD4, CD5, CD8, CD14, CD15, CD19, CD20, CD21, CD22, CD23, CD25, CD33, CD37, CD38, CD40, CD40L, CD46, CD47, CD54, CD67, CD74, CD79a, CD80, CD126, CD138, CD154, CD273 (PD-L2), CD274(PD-L1), CXCR4, B7, MUC1 or 1a, HM1.24, HLA-DR, tenascin, VEGF, P1GF, ED-B fibronectin, an oncogene, an oncogene product (e.g. c-Met or PLAGL2), CD66a-d, necrosis antigens, IL-2, T101, TAG, IL-6, MIF, TRAIL-R1 (DR4) and TRAIL-R2 (DR5).

Various bi-specific PEGylated antibody hydroxyl-bearing drug conjugate can be made against two different targets. Examples of the antigen pairs include CD19/CD3, BCMA/CD3, different antigens of the HER family in combination (EGFR, HER2, HER3), IL17RA/IL7R, IL-6/IL-23, IL-1-β/IL-8, IL-6 or IL-6R/IL-21 or IL-21R, ANG2/VEGF, VEGF/PDGFR-0, VEGF 2/CD3, PSMA/CD3, EPCAM/CD3, combinations of antigens selected from a group consisting of VEGFR-1, VEGFR-2, VEGFR-3, FLT3, c-FMS/CSF1R, RET, c-Met, EGFR, Her2/neu, HER3, HER4, IGFR, PDGFR, c-KIT, BCR, integrin and MMPs with a water-soluble ligand is selected from the group consisting of VEGF, EGF, PIGF, PDGF, HGF, and angiopoietin, ERBB-3cC-Met, ERBB-2/c-Met, EGF receptor 1/CD3, EGFR/HER3, PSCA/CD3, c-Met/CD3, ENDOSIALIN/CD3, EPCAM/CD3, IGF-1R/CD3, FAPALPHA/CD3, EGFR/IGF-1R, IL 17A/F, EGF receptor 1/CD3, and CD19/CD16. Additional examples of bispecific ADCs can have (i) a first specificity directed to a glycoepitope of an antigen selected from the group consisting of Lewis x-, Lewis b- and Lewis y-structures, Globo H-structures, KH1, Tn-antigen, TF-antigen and carbohydrate structures of Mucins, CD44, glycolipids and glycosphingolipids, such as Gg3, Gb3, GD3, GD2, Gb5, Gm1, Gm2, and sialyltetraosylceramide and (ii) a second specificity directed to an ErbB receptor tyrosine kinase selected from the group consisting of EGFR, HER2, HER3 and HER4. GD2 in combination with a second antigen binding site is associated with an immunological cell chosen from the group consisting of T-lymphocytes NK cell, B-lymphocytes, dendritic cells, monocytes, macrophages, neutrophils, mesenchymal stem cells, neural stem cells.

A monospecific or bispecific antibody can be joined together with another monospecific or bispecific antibody using the method disclosed herein to make multi-specific PEGylated ADCs, an additive/synergistic effect can be expected in comparison to the single targeting ADC.

In some embodiments, multi-specific PEGylated ADCs of this invention are made using antibody pairs that specifically interact and show measurable affinities to the following target pairs.

| Single chain antibody | | |
|---|---|---|
| fragments Targets | Mechanisms of action | Diseases (or healthy volunteers) |
| CD3, EpCAM | Retargeting of T cells to tumor, Fc | Malignant ascites in EpCAM |
| | mediated effector functions | positive tumors, Solid tumor |
| CD3, Her2 | Retargeting of T cells to tumor | Metastatic breast cancer, |
| | | Advanced solid tumors |
| CD3, CD19 | Retargeting of T cells to tumor | Precursor B-cell ALL, ALL, |
| | | DLBCL, NHL |
| CD3, CEA | Retargeting of T cells to tumor | Gastrointestinal |
| | | adenocancinoma |
| CD3, PSMA | Retargeting of T cells to tumor | Prostate cancer |
| CD3, CD123 | Retargeting of T cells to tumor | AML |
| CD3, gpA33 | Retargeting of T cells to tumor | Colorectal cancer |
| CD30, | Retargeting of NK cells to tumor | Hodgkin's Lymphoma |
| CD16A | cells | |
| CD3, GD2 | Retargeting of T cells to tumor | Neuroblastoma and |
| | | osteosarcoma |
| CD3, EGFR | Autologous activated T cells to | Lung and other solid tumors, |
| | EGFR-positive tumor | Colon and pancreatic cancers |
| CD28, MAPG | Retargeting of T cells to tumor | Metastatic melanoma |
| CD3, peptide | Retargeting of T cells to tumor | Metastatic melanoma |
| MHC | | |
| CD19, CD22 | Targeting of protein toxin to tumor | B cell leukemiaor lymphoma |
| EGFR, HER3 | Blockade of 2 receptors, ADCC | Head and neck cancer |
| | | Colorectal cancer |
| EGFR, c-Met | Blockade of 2 receptors | Advanced or metastatic |
| | | cancer |
| EGFR, 5T4 | Blockade of 2 receptors | Advanced or metastatic |
| | | cancer |
| EGFR, Trop2 | Blockade of 2 receptors | Advanced or metastatic |
| | | cancer |
| EGFR, CD73 | Blockade of 2 receptors | Advanced or metastatic |
| | | cancer |
| EGFR, | Blockade of 2 receptors | Advanced or metastatic |
| Nectin4 | | cancer |
| EGFR, Cripto 1 | Blockade of 2 receptors | Advanced or metastatic |
| | | cancer |
| cMet, 5T4 | Blockade of 2 receptors | Advanced or metastatic |
| | | cancer |
| cMet, Trop2 | Blockade of 2 receptors | Advanced or metastatic |
| | | cancer |
| cMet, nectin4 | Blockade of 2 receptors | Advanced or metastatic |
| | | cancer |
| cMet, Cripto 1 | Blockade of 2 receptors | Advanced or metastatic |
| | | cancer |
| cMet, Her3 | Blockade of 2 receptors | Advanced or metastatic |
| | | cancer |
| cMet, CEA | Blockade of 2 receptors | Advanced or metastatic |
| | | cancer |
| cMet, CD73 | Blockade of 2 receptors | Advanced or metastatic |
| | | cancer |
| 5T4, CEA | Blockade of 2 receptors | Advanced or metastatic |
| | | cancer |
| 5T4, Trop2 | Blockade of 2 receptors | Advanced or metastatic |
| | | cancer |
| 5T4, Cripto 1 | Blockade of 2 receptors | Advanced or metastatic |
| | | cancer |
| 5T4, Nectin4 | Blockade of 2 receptors | Advanced or metastatic |
| | | cancer |
| 5T4, CEA | Blockade of 2 receptors | Advanced or metastatic |
| | | cancer |
| HER2, HER2 | Blockade of 2 same or different | Gastric and esophageal |
| | receptors | cancers Breast cancer |
| HER2, HER3 | Blockade of 2 receptors | Gastric and esophageal |
| | | cancers Breast cancer |
| IGF-1R, | Blockade of 2 receptors | Advanced solid tumors |
| HER3 | | |
| Ang2, VEGFA | Blockade of 2 proangiogenics | Solid tumors, Wet AMD |
| CEA, HSG | Pretargeting tumor for PET or radio | Colorectal<comma> breast |
| | imaging | and lung cancers |
| IL-1α, IL-1β | Blockade of 2 proinflammatory | Osteoarthritis |
| | cytokines | |
| TNF, IL-17A | Blockade of 2 proinflammatory | Rheumatoid arthritis, Plaque |
| | cytokines | psoriasis |
| IL-13, IL-4 | Blockade of 2 proinflammatory | Idiopathic pulmonary fibrosis |
| | cytokines | |
| IL-13, IL-4 | Blockade of 2 proinflammatory | (Healthy volunteers) |
| | cytokines | |
| TNF, HSA | Blockade of proinflammatory | Rheumatoid arthritis |
| | cytokine, binds to HSA to increase | |
| | half-life | |
| IL-17A/F, | Blockade of 2 proinflammatory | (Healthy volunteers) |
| HSA | cytokines, binds to HSA to increase | |
| | half-life | |
| IL-6R, HSA | Blockade of proinflammatory | Rheumatoid arthritis |
| | cytokine, binds to HSA to increase | |
| | half-life | |
| RANKL, HSA | Blockade of bone resorption, binds | Postmenopausal bone loss |
| | to HSA to increase half-life | |
| Factor Ixa, | Plasma coagulation | Hemophilia |
| factor X | | |
| IL-6R, HSA | Blockade of proinflammatory | Rheumatoid arthritis |
| | cytokine, binds to HSA to increase | |
| | half-life | |
| RANKL, HSA | Blockade of bone resorption, binds | Postmenopausal bone loss |
| | to HSA to increase half-life | |
| Factor Ixa, | Plasma coagulation | Hemophilia |
| factor X | | |

In some embodiment, a PEGylated BsADC comprises a bispecific single chain antibody, wherein the two binding domains of the bispecific single chain antibody are linked via a peptide linker. In some embodiments, the peptide linker comprises a moiety such as cysteine or an unnatural amino acid residue that can be used for site-specific conjugation of the antibody to a non-immunogenic polymer hydroxyl-bearing drug conjugate, e.g. PEGylated hydroxyl-bearing drug conjugate. In some other embodiments, one or both of the two binding domains of the bispecific single chain antibody comprises a cysteine or an unnatural amino acid residue that can be used for site-specific conjugation of the antibody to a non-immunogenic polymer hydroxyl-bearing drug conjugate, e.g. PEGylated hydroxyl-bearing drug conjugate.

In a preferred embodiment, a PEGylated bi-specific hydroxyl-bearing drug conjugate is a conjugate of two antibodies or antigen-binding fragments (such as Fabs, scFvs, nanobody and the like) thereof that specifically interact and show measurable affinities to two different epitopes of Her2.

### IX. Synthesis

Once the desired size and shape of PEG have been selected, the terminal functional group of PEG such as hydroxyl, carboxyl group and the like can be converted to terminal branched heterobifunctional groups using any art-recognized process (WO2018075308). Broadly stated, the terminal branched heterobifunctional PEG can be prepared by activating terminal hydroxyl or carboxyl group of the PEG with N-Hydroxysuccinimide using reagents such as Di(N-succinimidyl) carbonate (DSC), triphosgene and the like in the case of terminal hydroxyl group or using coupling reagents such as N,N-Diisopropylcarbodiimide (DIPC), 1-Ethyl-3-(3-dimethylaminopropyl)carbodiimide (EDC) and the like in the case of terminal carboxyl group in the presence of base such as 4-Dimethylaminopyridine (DMAP), pyridine and the like to form an activated PEG.

Next, the activated PEG can be reacted with a multi-functional small molecule such as lysine derivative H-Lys(Boc)-OH in the presence of base such as Diisopropylamine (DIPEA) to form a terminal branched heterobifunctional PEG with a free carboxyl group and a Boc-protected amino group PEG-Lys(Boc)-COOH. As will be appreciated by those of ordinary skill, other terminal functional groups of PEG such as halide, amino, thiol group and the like and other trifunctional small molecules containing any combination of three functional groups from the list of —NH₂, —NHNH₂, —COOH, —OH, —C(O)X (X=halides), —N═C═O, —SH, anhydride, halides, maleimido, C=C, C≡C and the like or their protected version can be used as alternatives for the same purpose if desired.

Removal of Boc by TFA followed by reaction with a maleimide tagged spacer such as NHS-PEG₂-Maleimide forms PEG-Lys(Mal)-COOH.

Separately, the cytotoxic drug (e.g. SN38) linked to a trigger (e.g. val-cit) via two self-immolating spacers (e.g. PAB-EDA) is coupled to a branch unit with an extension with the coupling reagent such as EDCI/HOBt to produce compound B-D. e.g.

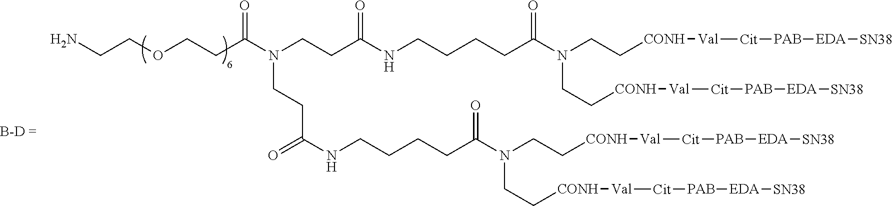

Target product could be formed by coupling PEG-Lys(Mal)-COOH with B-D with coupling reagent such as DCC to form PEGylated drug conjugate PEG-Lys(Mal)-4(Val-Cit-PAB-EDA-SN38).

Monospecific antibodies that is bivalent for the antigens or Bispecific antibodies such as SCAHer2IIxSCAHer2IV can be prepared through genetic manipulation of expression systems. For example, DNA encoding a bispecific scFv can be synthesized and introduced into an expression system (e.g, CHO cells). The protein of interest is then expressed and purified through chromatography technologies.

To prepare a PEGylated single chain antibody hydroxyl-bearing drug conjugate, the PEGylated hydroxyl-bearing drug conjugate with a functional group maleimide or DBCO can be reacted site specifically with free thiol or azide functional group of a bifunctional antibody [such as SCAPDL1xSCACD47 or SCAHer2(1)xSCAHer2(2) or SCAcMet(1)xcMet(2)] that is either genetically inserted or through derivatization of the protein, to form PEG-Lys(SCAPDL1xSCACD47)-4(Val-Cit-PAB-EDA-SN38) or PEG-Lys(SCAHer2(1)xSCAHer2(2))-4(Val-Cit-PAB-EDA-SN38) or PEG-Lys(SCAcMet(1)xSCAcMet(2))-4(Val-Cit-PAB-EDA-SN38).

PEGylated multi-specific hydroxyl-bearing antibody drug conjugate can be prepared similarly using multi-specific antibody instead of mono- or bispecific antibody.

In addition to thiol/maleimide or DBCO/azide site-specific conjugation group pair exemplified in this invention, as will be appreciated by those of ordinary skill, other known pairs of site-specific conjugation groups, such as trans-cyclooctenes/tetrazines pair; carbonyl/hydrazide; carbonyl/oxime; Suzuki-Miyaura cross-coupling reagent pair; Sonogashira coss-coupling reagent pair; Staudinger ligation reagent pair; Knoevenagel-intra Michael addition reagent pair, active amine/acrylate pair and the like can be similarly designed and used as alternatives for the same purpose if desired. The foregoing list of site-specific conjugation group pairs is merely illustrative and not intended to restrict the type of site-specific conjugation group pairs suitable for use herein.

### X. Compositions

The present invention also provides a composition, e.g., a pharmaceutical composition, containing the compound of the present invention, formulated together with a pharmaceutically acceptable carrier. For example, a pharmaceutical composition of the invention can comprise a compound (e.g. a PEGylated bispecific hydroxyl-bearing antibody-drug conjugate) that binds to two different of epitopes of Her2 receptor.

Therapeutic formulations of this invention can be prepared by mixing the mono- or multi-specific molecule drug conjugate having the desired degree of purity with optional physiologically acceptable carriers, excipients or stabilizers in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates; chelating agents such as EDTA; sugars such as sucrose, mannitol, trehalose or sorbitol; salt-forming counter-ions such as sodium; metal complexes (e.g. Zn-protein complexes); and/or non-ionic surfactants such as Tween, Pluronics, or PEG.

The formulation may also contain more than one active compound as necessary for the particular indication being treated, preferably those with complementary activities that do not adversely affect each other. For instance, the formulation may further comprise another antibody or multi-specific antibody, cytotoxic agent, chemotherapeutic agent or ADC. Such molecules are suitably present in combination in amounts that are effective for the purpose intended.

Pharmaceutical compositions of the invention can be administered in combination therapy, i.e., combined with other agents. Examples of therapeutic agents that can be used in combination therapy are described in greater detail below.

The formulations to be used for in vivo administration must be sterile. This can be readily accomplished by filtration through sterile filtration membranes. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

### XI. Dosage

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01% to about 99% of active ingredient, preferably from about 0.1% to about 70%, most preferably from about 1% to about 50% of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the PEGylated mono- or multi-specific hydroxyl-bearing drug conjugate of this invention, the dosage ranges from about 0.0001 to 100 mg/kg, and more usually 0.01 to 50 mg/kg, of the host body weight. For example dosages can be 0.1 mg/kg body weight, 1 mg/kg body weight, 5 mg/kg body weight, 10 mg/kg body weight or 15 mg/kg body weight or within the range of 1-15 mg/kg. An exemplary treatment regime entails administration daily, once every other day, twice per week, once per week, once every two weeks, once every three weeks, once every four weeks, once a month, once every 3 months or once every 6 months. Preferred dosage regimens for PEGylated mono- or multi-specific hydroxyl-bearing drug conjugate of the invention include 1 mg/kg body weight or 3 mg/kg body weight via intravenous administration, with the mono- or multi-specific drug conjugate being given using one of the following dosing schedules: (i) every three weeks for six dosages, then one dosage every month; (ii) one dosage every three weeks; (iii) 3 mg/kg body weight once followed by 1 mg/kg body weight every three weeks.

Dosage and frequency vary depending on the half-life of the mono- or multi-specific drug conjugate in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a PEGylated mono- or multi-specific hydroxyl-bearing drug conjugate of the invention preferably results in a decrease in severity of disease symptoms, an increase in frequency and duration of disease symptom-free periods, or a prevention of impairment or disability due to the disease affliction. For example, for the treatment of tumors, a “therapeutically effective dosage” preferably inhibits cell growth or tumor growth or metastasis by at least about 10%, more preferably by at least about 40%, even more preferably by at least about 60%, and still more preferably by at least about 80% relative to untreated subjects. The ability of an agent or compound to inhibit tumor growth can be evaluated in an animal model system predictive of efficacy in human tumors. Alternatively, this property of a composition can be evaluated by examining the ability of the compound to inhibit in vitro by assays known to the skilled practitioner. A therapeutically effective amount of a therapeutic compound can decrease tumor size, metastasis, or otherwise ameliorate symptoms in a subject. One of ordinary skill in the art would be able to determine such amounts based on such factors as the subject's size, the severity of the subject's symptoms, and the particular composition or route of administration selected.

### XII. Administration

A composition of the invention can be administered via one or more routes of administration using one or more of a variety of methods known in the art. As will be appreciated by the skilled artisan, the route and/or mode of administration will vary depending upon the desired results. Preferred routes of administration for PEGylated mono- or multi-specific hydroxyl-bearing drug conjugate of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a mono- or multi-specific drug conjugate of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, and 4,596,556. Examples of well-known implants and modules useful in the present invention include those described in U.S. Pat. Nos. 4,487,603, 4,486,194, 4,447,233, 4,447,224, 4,439,196, and 4,475,196. These patents are incorporated herein by reference. Many other such implants, delivery systems, and modules are known to those skilled in the art.

### XIII. Treatment

The PEGylated mono- or multi-specific hydroxyl-bearing drug conjugate disclosed herein can be used in the preparation of medicaments for the treatment of an oncologic disease, a cardiovascular disease, an infectious disease, an inflammatory disease, an autoimmune disease, a metabolic (e.g., endocrine) disease, or a neurological (e.g., neurodegenerative) disease. Exemplary non-limiting examples of these diseases are Alzheimer's disease, non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, carcinomas (such as carcinomas of the oral cavity, gastrointestinal tract, colon, stomach, pulmonary tract, lung, breast, ovary, prostate, uterus, endometrium, cervix, urinary bladder, pancreas, bone, liver, gall bladder, kidney, skin, and testes), melanomas, sarcomas, gliomas, and skin cancers, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, or fibrosing alveolitis.

In one aspect, the present invention relates to treatment of a subject in vivo using the above-described PEGylated mono- or multi-specific hydroxyl-bearing drug conjugate such that growth and/or metastasis of cancerous tumors is inhibited. In one embodiment, the invention provides a method of inhibiting growth and/or restricting metastatic spread of tumor cells in a subject, comprising administering to the subject a therapeutically effective amount of a mono-or multi-specific molecule drug conjugate.

Non-limiting examples of preferred cancers for treatment include chronic or acute leukemia including acute myeloid leukemia, chronic myeloid leukemia, acute lymphoblastic leukemia, chronic lymphocytic leukemia, lymphocytic lymphoma, breast cancer, ovarian cancer, melanoma (e.g., metastatic malignant melanoma), renal cancer (e.g. clear cell carcinoma), prostate cancer (e.g. hormone refractory prostate adenocarcinoma), colon cancer and lung cancer (e.g. non-small cell lung cancer). Additionally, the invention includes refractory or recurrent malignancies whose growth may be inhibited using the antibodies of the invention. Examples of other cancers that may be treated using the methods of the invention include bone cancer, pancreatic cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

As used herein, the term “subject” is intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients in need of enhancement of an immune response. The methods are particularly suitable for treating human patients having a disorder that can be treated by augmenting the immune response.

The above treatment may also be combined with standard cancer treatments. For example, it may be effectively combined with chemotherapeutic regimes. In these instances, it may be possible to reduce the dose of chemotherapeutic reagent administered (Mokyr, M. et al. Cancer Res., 1998, 58, 5301-5304).

Other antibodies which may be used to activate host immune responsiveness can be used with PEGylated mono- or multi-specific hydroxyl-bearing drug conjugate of this invention. These include molecules targeting on the surface of dendritic cells which activate DC function and antigen presentation. For example, anti-CD40 antibodies are able to substitute effectively for T cell helper activity (Ridge, J. et al., Nature, 1998, 393, 474-478) and can be used in conjunction with mono- or multi-specific drug conjugate of this invention (Ito, N. et al., Immunobiology, 2000, 201, 527-540). Similarly, antibodies targeting T cell costimulatory molecules such as CTLA-4 (U.S. Pat. No. 5,811,097), CD28 (Haan, J. et al., Immunol. Lett., 2014, 162, 103-112), OX-40 (Weinberg, A. et al., J. Immunol., 2000, 164, 2160-2169), 4-1BB (Melero, I. et al., Nature Med., 1997, 3, 682-685), and ICOS (Hutloff, A. et al., Nature, 1999, 397, 262-266) or antibodies targeting PD-1 (U.S. Pat. No. 8,008,449) and PD-L1 (U.S. Pat. Nos. 7,943,743; 8,168,179) may also provide for increased levels of T cell activation. In another example, the mono- or multi-specific drug conjugate of this invention can be used in conjunction with anti-neoplastic antibodies, such as Rituxan (rituximab), Herceptin (trastuzumab), Bexxar (tositumomab), Zevalin (ibritumomab), Campath (alemtuzumab), Lymphocide (eprtuzumab), Avastin (bevacizumab), and Tarceva (erlotinib), and the like.

### Definitions of Terms

The term “alkyl” as used herein refers to a hydrocarbon chain, typically ranging from about 1 to 25 atoms in length. Such hydrocarbon chains are preferably but not necessarily saturated and may be branched or straight chain, although typically straight chain is preferred. The term C₁₋₁₀ alkyl includes alkyl groups with 1, 2, 3, 4, 5, 6, 7, 8, 9 and 10 carbons. Similarly C₁₋₂₅ alkyl includes all alkyls with 1 to 25 carbons. Exemplary alkyl groups include methyl, ethyl, isopropyl, n-butyl, n-pentyl, 2-methyl-1-butyl, 3-pentyl, 3-methyl-3-pentyl, and the like. As used herein, “alkyl” includes cycloalkyl when three or more carbon atoms are referenced. Unless otherwise noted, an alkyl can be substituted or unsubstituted.

The term “functional group” as used herein refers to a group that may be used, under normal conditions of organic synthesis, to form a covalent linkage between the entity to which it is attached and another entity, which typically bears a further functional group. A “bifunctional linker” refers to a linker with two functional groups that can form two linkages with other moieties of a conjugate.

The term “derivative” as used herein refers to a chemically-modified compound with an additional structural moiety for the purpose of introducing new functional group or tuning the properties of the original compound.

The term “protecting group” as used herein refers to a moiety that prevents or blocks reaction of a particular chemically reactive functional group in a molecule under certain reaction conditions. Various protecting groups are well-known in the art and are described, for example, in T. W. Greene and G. M. Wuts, Protecting Groups in Organic Synthesis, Third Edition, Wiley, New York, 1999, and in P. J. Kocienski, Protecting Groups, Third Ed., Thieme Chemistry, 2003, and references cited therein.

The term “PEG” as used herein refers to polyethylene glycol. PEGs for use in the present invention typically comprise a structure of —(CH₂CH₂O)ₙ—. PEGs may have a variety of molecular weights, structures or geometries. A PEG group may comprise a capping group that does not readily undergo chemical transformation under typical synthetic reaction conditions. Examples of capping groups include —OC₁₋₂₅ alkyl or -OAryl.

The term “PEGylate” as used herein refers to chemical modification by polyethylene glycol.

The term “linker” as used herein refers to an atom or a collection of atoms used to link interconnecting moieties, such as an antibody and a cytotoxic drug. A linker can be cleavable or noncleavable. The preparation of various linkers for conjugates have been described in literatures including for example Goldmacher et al., Antibody-drug Conjugates and Immunotoxins: From Pre-clinical Development to Therapeutic Applications, Chapter 7, in Linker Technology and Impact of Linker Design on ADC properties, Edited by Phillips GL; Ed. Springer Science and Business Media, New York (2013). Cleavable linkers incorporate groups or moieties that can be cleaved under certain biological or chemical conditions. Examples include enzymatically cleavable valine citrulline amino sequence, disulfide linkers, 1,4- or 1,6-benzyl elimination, trimethyl lock cleavable system, bicine-based self-cleavable system, acid-labile silyl ether linkers and photo-labile linkers.

The term “linking group” or “linkage” as used herein refers to a functional group or moiety connecting different moieties of a compound or conjugate. Examples of a linking group include, but are not limited to, amide, ester, carbamate, ether, thioether, disulfide, hydrazone, oxime, and semicarbazide, carbodiimide, acid labile group, photolabile group, peptidase labile group and esterase labile group. For example, a linker moiety and a polymer moiety may be connected to each other via an amide or carbamate linking group.

The terms “peptide,” “polypeptide,” and “protein” are used herein interchangeably to describe the arrangement of amino acid residues in a polymer. A peptide, polypeptide, or protein can be composed of the standard 20 naturally occurring amino acid, in addition to rare amino acids and synthetic amino acid analogs. They can be any chain of amino acids, regardless of length or post-translational modification (for example, glycosylation or phosphorylation).

A “recombinant” peptide, polypeptide, or protein refers to a peptide, polypeptide, or protein produced by recombinant DNA techniques; i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

An “isolated” peptide, polypeptide, or protein refers to a peptide, polypeptide, or protein that has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide/protein described in the invention can be purified from a natural source, produced by recombinant DNA techniques, or by chemical methods.

An “antigen” refers to a substance that elicits an immunological reaction or binds to the products of that reaction. The term “epitope” refers to the region of an antigen to which an antibody or T cell binds.

The term “antibody” as referred to herein includes whole antibodies and any antigen binding fragment or single chains thereof. Whole antibodies are glycoproteins comprising at least two heavy (H) chains and two light (L) chains inter-connected by disulfide bonds. Each heavy chain is comprised of a heavy chain variable region (V_{H}) and a heavy chain constant region. The heavy chain constant region is comprised of three domains, C_{H}1, C_{H}2 and C_{H}3. Each light chain is comprised of a light chain variable region (V_{L}) and a light chain constant region (C_{L}), the light chain constant region is comprised of one domain. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

As used herein, “antibody fragments”, may comprise a portion of an intact antibody, generally including the antigen binding and/or variable region of the intact antibody and/or the Fc region of an antibody which retains FcR binding capability. Examples of antibody fragments include linear antibodies; single-chain antibody molecules; nanobody; and multispecific antibodies formed from antibody fragments.

The term “antigen-binding fragment or portion” of an antibody (or simply “antibody fragment or portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the V_{L}, V_{H}, C_{L} and C_{H}I domains; (ii) a F(ab′)2 fragment, a bivalent fragment comprising two Fab fragments linked by a disulfide bridge at the hinge region; (iii) a Fab′ fragment, which is essentially an Fab with part of the hinge region; (iv) a Fd fragment consisting of the V_{H} and C_{H}I domains; (v) a Fv fragment consisting of the VL and VH domains of a single arm of an antibody, (vi) a dAb, which consists of a VH domain; (vii) an isolated complementarity determining region (CDR); and (viii) a nanobody, a heavy chain variable region containing a single variable domain and two constant domains. Furthermore, although the two domains of the Fv fragment, VL and V_{H}, are coded for by separate genes, they can be joined, using recombinant methods, by a synthetic linker that enables them to be made as a single protein chain in which the VL and V_{H} regions pair to form monovalent molecules (known as single chain Fv (scFv); see e.g., Bird, R. E. et al., Science, 1988, 242, 423-426; and Huston, J. S. et al., Proc. Nat. Acad. Sci. USA, 1988, 85, 5879-5883. Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

As used herein, the term “Fc fragment” or “Fc region” is used to define a C-terminal region of an immunoglobulin heavy chain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein (Kohler, G. et al., Nature, 1975, 256, 495-497), which is incorporated herein by reference, or may be made by recombinant DNA methods (U.S. Pat. No. 4,816,567), which is incorporated herein by reference. The monoclonal antibodies may also be isolated from phage antibody libraries using the techniques described by Clackson T. et al., Nature, 1991, 352, 624-628 and Marks J. D. et al., J Mol Biol, 1991, 222, 581-597, for example, each of which is incorporated herein by reference.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; Morrison, S. L. et al., Proc. Natl. Acad. Sci. USA, 1984, 81, 6851-6855; Neuberger, M. S. et al., Nature, 1984, 312, 604-608; Takeda, S. et al., Nature, 1985, 314, 452-454; PCT/GB8500392, each of which is incorporated herein by reference).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody optionally also will comprise at least a portion of an immunoglobulin constant region (Fc), typically that of a human immunoglobulin. For further details, Jones, P. T. et al., Nature, 1986, 321, 522-525; Riechmann, L. et al., Nature, 1988, 332, 323-329; Presta, L. G. Curr. Opion. Struct. Biol., 1992, 2, 593-596; U.S. Pat. No. 5,225,539, each of which is incorporated herein by reference.

“Human antibodies” refer to any antibody with fully human sequences, such as might be obtained from a human hybridoma, human phage display library or transgenic mouse expressing human antibody sequences.

The term “pharmaceutical composition” refers to the combination of an active agent with a carrier, inert or active, making the composition especially suitable for diagnostic or therapeutic use in vivo or ex vivo.

As used herein, “pharmaceutically acceptable carrier” includes any and all solvents, dispersion media, coatings, antibacterial and antifungal agents, isotonic and absorption delaying agents, and the like that are physiologically compatible. A “pharmaceutically acceptable carrier”, after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. Preferably, the carrier is suitable for intravenous, intramuscular, subcutaneous, parenteral, spinal or epidermal administration (e.g., by injection or infusion). The therapeutic compounds may include one or more pharmaceutically acceptable salts. A “pharmaceutically acceptable salt” refers to a salt that retains the desired biological activity of the parent compound and does not impart any undesired toxicological effects (see e.g. Berge, S. M. et al., J. Pharm. Sci., 1997, 66,1-19).

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

An “effective amount” refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment. A therapeutically effective amount of a combination to treat a neoplastic condition is an amount that will cause, for example, a reduction in tumor size, a reduction in the number of tumor foci, or slow the growth of a tumor, as compared to untreated animals.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

### EXAMPLES

The following examples serve to provide further appreciation of the invention but are not meant by any way to restrict the effective scope of the invention.

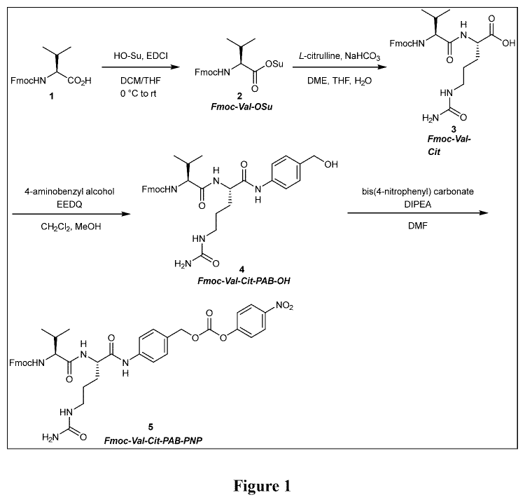

### Example 1. Preparation of 30kmPEG-Lys(Mal)-3(Val-Cit-PAB-DEA-SN38) Preparation of Fmoc-Val-Cit-PAB-PNP (Compound 5, FIG. 1)

Fmoc-Val-OSu (2): Fmoc-Val-OH (20.3 g, 60.0 mmol) and N-hydroxysuccinimide (NHS, 9.0 g, 78.0 mmol) were dissolved in a mixture of CH₂Cl₂ (120 mL) and THF (40 mL). Separately, EDCI (13.8 g, 72.0 mmol) was dissolved in CH₂Cl₂ (200 mL) and cooled to 0-5° C. The solution of Fmoc-Val-OH and NHS was then added to the EDCI solution. The reaction was allowed to warm up to room temperature and stirred at room temperature until reaction was completed. The reaction mixture was then concentrated under reduced pressure and azeotropically distilled twice with THF (100 mL). The concentrated residue was dissolved with THF (800 mL) and filtered to remove EDU. The filtrate was concentrated under reduced pressure and re-slurried with n-heptane (800 mL) at 5-10° C. for 12 h. Solids were filtered, washed and dried under vacuum to yield Fmoc-Val-OSu (2) (23.8 g, 91%) as white powder. HRMS (ESI) calcd for C₂₄H₂₄N₂O₆Na [M+Na]⁺ 459.1532, found 459.1523.

Fmoc-Val-Cit (3): Fmoc-Val-OSu (2) (9.8 g, 22.5 mmol) was dissolved in DME (150 mL) at room temperature. Separately, sodium bicarbonate (2.1 g, 24.7 mmol) was dissolved in water (150 mL) at room temperature followed by the addition of L-citrulline (4.3 g, 24.7 mmol) to give a homogeneous clear solution. The L-citrulline solution prepared was then added to the Fmoc-Val-OSu solution, followed by the addition of THF (75 mL). The reaction mixture was stirred at room temperature for 16 h. After reaction was completed, the mixture was acidified with 15% citric acid (200 mL), then concentrated under vacuum. The mixture was suspended in water (500 mL) and the resulting mixture was stirred for 2 h followed by filtration and drying under vacuum. The dried solids were re-suspended in methyl tert-butyl ether (500 mL) and stirred for 12 h. The suspension was filtered and washed. The isolated solids were dried under vacuum to yield Fmoc-Val-Cit (3) (6.8 g, 61%) as white powder. HRMS (ESI) calcd for C₂₆H₃₃N₄O₆ [M+H]⁺ 497.2400, found 497.2388.

Fmoc-Val-Cit-PAB-OH (4): EEDQ (4.95 g, 20.0 mmol) was added to the solution of compound 3 (4.96 g, 10.0 mmol) and 4-aminobenzyl alcohol (2.46 g, 20.0 mmol) in CH₂Cl₂ (350 mL) and MeOH (150 mL). The reaction mixture was stirred at room temperature for 24 h. Additional EEDQ (2.5 g, 10.0 mmol) was added and stirred for another 24 h. After the reaction was completed, the solvent was removed under reduced pressure and the resulting residue was re-slurred in methyl tert-butyl ether (800 mL) for 12 h. Solids were filtered, washed and dried under vacuum to yield compound 4 (4.1 g, 69%) as white powder. HRMS (ESI) calcd for C₃₃H₄₀N₅O₆ [M+H]⁺ 602.2979, found 602.2969.

Fmoc-Val-Cit-PAB-PNP (5): DIPEA (2.5 mL, 15.0 mmol) was added to a solution of compound 4 (5.2 g, 8.6 mmol) and bis(4-nitrophenyl) carbonate (4.9 g, 16.1 mmol) in DMF (52 mL) at room temperature. The reaction mixture was stirred at room temperature for 5 h. After reaction was completed, the product was precipitated out of the reaction mixture by adding anhydrous ethyl acetate (250 mL) and methyl tert-butyl ether (250 mL). The resulting slurry was stirred and cooled to 0° C. After 30 min stirring at 0° C., the solids were isolated by filtration followed by washing and drying under vacuum to yield Fmoc-Val-Cit-PAB-PNP (5) (4.7 g, 72%) as pale yellow powder. HRMS (ESI) calcd for C₄₀H₄₃N₆O₁₀ [M+H]⁺ 767.3041, found 767.3045.

### Preparation of Val-Cit-PAB-DEA-SN38 (Compound 10)

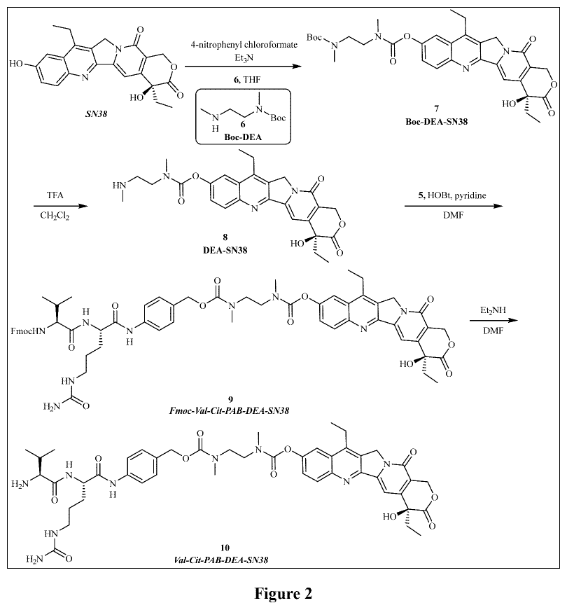

### Reaction Scheme a (FIG. 2)

Boc-DEA-SN38 (7): A solution of SN-38 (3.9 g, 10 mmol) in anhydrous THF (100 mL) is cooled to 0° C. under a nitrogen atmosphere, after which 4-nitrophenyl chloroformate (2.7 g, 13.4 mmol) and Et₃N (7.0 mL, 50 mmol) are added. The mixture is stirred at 0° C. for 1.5 h. Boc-DEA (6) (9.4 g, 50 mmol) is added and the mixture is stirred for another 1 h. The reaction is slowly being warmed to room temperature. After reaction is completed, the reaction mixture is t concentrated, and the crude product is purified by column chromatography to yield the compound 7.

DEA-SN38 (8): A solution of compound 7 (0.99 g, 1.63 mmol) in CH₂Cl₂ (10 mL) is cooled to 0° C., after which TFA (3 mL) is added. The mixture is stirred at 0° C. for 1 h, followed by addition of CH₂Cl₂ (10 mL). The diluted mixture is concentrated to yield crude product 8.

Fmoc-Val-Cit-PAB-DEA-SN38 (9): Compound 8 (1.4 g, 2.8 mmol) and Fmoc-Val-Cit-PAB-PNP (5) (2.8 g, 3.6 mmol) are dissolved in DMF (20 m L). HOBt (0.75 g, 5.6 mmol) and pyridine (1.7 mL) are then added and the reaction mixture is stirred at room temperature for 24 h. After the reaction is completed, the reaction mixture is cooled to 0° C. and methyl tert-butyl ether (180 mL) is added. The resultant slurry is stirred for 3-5 h and filtered. The isolated solids are washed and dried under vacuum. The crude product is purified by column purification to afford compound 9.

Val-Cit-PAB-DEA-SN38 (10): Compound 9 (2.5 g, 2.2 mmol) is suspended in anhydrous DMF (40 mL) and the resulting suspension is stirred at room temperature until a homogeneous suspension is formed. Diethylamine (10 mL) is then added and the reaction mixture is stirred at room temperature for 3 h. After reaction is completed, Methyl tert-butyl ether (100 mL) and ethyl acetate (50 mL) are then added over 60 min. The resulting mixture is stirred for 4 h at 0° C. Solids are filtered and dried under vacuum to yield compound 10.

### Reaction Scheme B (FIG. 3)

Boc-DEA-SN38 (7): A solution of SN-38 (11.8 g, 30 mmol) and DIPEA (18.3 mL, 105 mmol) in anhydrous CH₂Cl₂ (500 mL) was cooled to 0° C. under a nitrogen atmosphere, after which 4-nitrophenyl chloroformate (19.3 g, 960 mmol) were added. The mixture was stirred at room temperature for 16 h. After the reaction was complete, the solvent was removed under reduced pressure and the resulting residue was re-slurried in methyl tert-butyl ether (600 mL) for 1 h. Solids were filtered, washed and dried under vacuum to yield compound PNP-SN38 (16 g, 96%) as pale yellow powder. MS (ESI) m/z [M+H]⁺ 558.29.

Compound 6 (5.31 g, 27.0 mmol) and PNP-SN38 (5.02 g, 9.0 mmol) were solubilized in DMF (50 mL). HOBt (2.43 g, 18.0 mmol) and pyridine (4.51 mL) were then added and the reaction mixture was stirred at room temperature for 24 h until the reaction was complete. The reaction mixture was cooled to 0° C. and was added to methyl tert-butyl ether (90 mL). The resultant slurry was stirred for 3-5 h and filtered, washed and dried under vacuum. The crude product was purified by column purification to yield compound 7 (4.5 g, 82%) as pale yellow powder. MS (ESI) m/z [M+H]⁺ 607.30, [M+Na]⁺ 629.30.

DEA-SN38 (8): A solution of compound 7 (3.03 g, 5.0 mmol) in CH₂Cl₂ (20 mL) was stirred at room temperature, TFA (4 mL) was added dropwise. The mixture was stirred at room temperature for 0.5 h, the solvent was removed under vacuum. The residue was treated with methyl tert-butyl ether (60 mL), the resultant slurry was stirred for 1 h and filtered, washed and dried to give the pure compound 8 (2.42 g, 96%) as pale yellow solid. MS (ESI) m/z [M+Na]+529.25.

Fmoc-Val-Cit-PAB-DEA-SN38 (9): Compound 8 (1.82 g, 3.6 mmol) and Fmoc-Val-Cit-PAB-PNP (5) (2.3 g, 3.0 mmol) were dissolved in DMF (20 m L). HOBt (0.81 g, 6.0 mmol) and pyridine (1.9 mL) were then added and the reaction mixture was stirred at room temperature for 24 h. After the reaction was completed, the reaction mixture was cooled to 0° C. and methyl tert-butyl ether (150 mL) was added. The resultant slurry was stirred for 1 h and filtered. The isolated solids were washed and dried under vacuum. The crude product was purified by column purification to afford compound 9 (2.9 g, 86%) as white solid. MS (ESI) m/z [M+H]⁺ 1134.57, [M+Na]⁺ 1156.47.

Val-Cit-PAB-DEA-SN38 (10): Compound 9 (2.5 g, 2.2 mmol) was suspended in anhydrous DMF (40 mL) and the resulting suspension was stirred at room temperature until a homogeneous suspension was formed. Diethylamine (10 mL) was then added and the reaction mixture was stirred at room temperature for 1 h. After reaction was completed, Methyl tert-butyl ether (100 mL) and ethyl acetate (50 mL) were then added over 60 min. The resulting mixture was stirred for 4 h at 0° C. Solids are filtered and dried under vacuum to yield compound 10 (2.0 g, 97%) as pale yellow powder. MS (ESI) m/z [M+H]⁺ 912.53, [M+Na]⁺ 934.43.

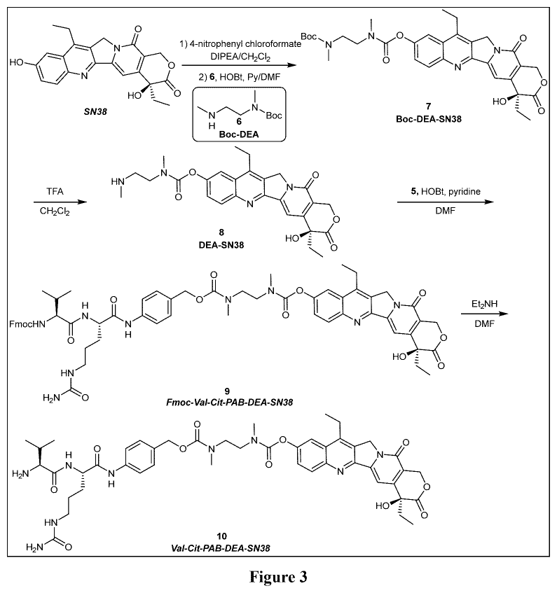

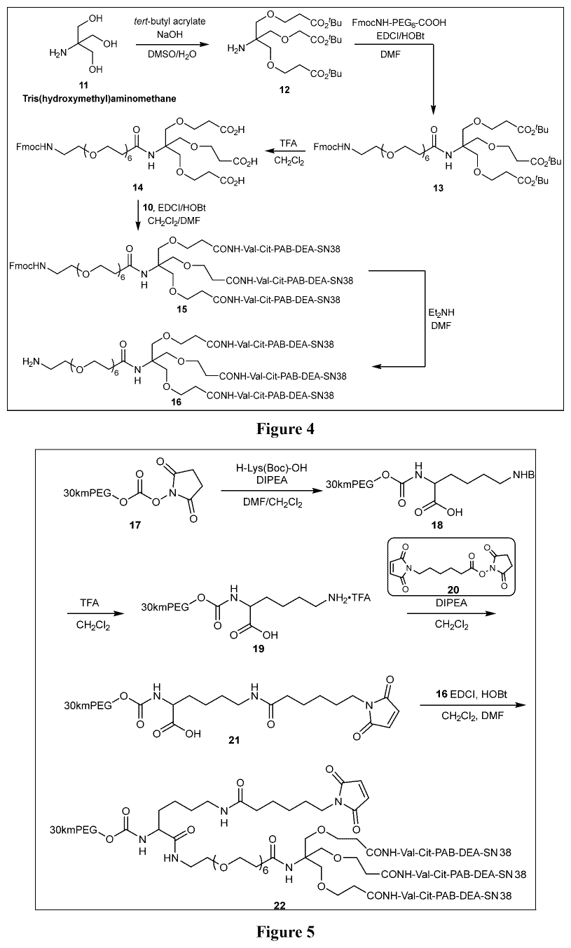

Preparation of Branched Intermediate NH₂-PEG₆-3(Val-Cit-PAB-DEA-SN38) (Compound 16, FIG. 4)

### Compound 12:

Reaction scheme A: The compound 11 (1.21 g, 10.0 mmol) in 2.0 mL of a newly opened bottle of DMSO is cooled to 15° C. under N₂. 0.2 mL of 5.0 M NaOH is injected while stirring, followed by injection of tert-butyl acrylate (5.0 mL, 34 mmol) dropwise (Note: A solvent mixture of 5-10% water in DMSO is optimal for this reaction). The reaction mixture is allowed to reach room temperature and left stirring for 24 h. At this point, the excess reagent and solvent are removed under vacuum at room temperature and the residue is purified by column chromatography to yield the compound 12.

Reaction scheme B: The compound 11 (2.43 g, 20.0 mmol) dissolved in 6.0 mL of a newly opened bottle of DMSO was cooled to 15° C. under argon. 0.6 mL of 5.0 M NaOH was injected while stirring, followed by injection of tert-butyl acrylate (8.72 g, 68 mmol) dropwise. The reaction mixture was allowed to reach room temperature and left stirring for 24 h. At this point, the excess reagent and solvent were removed under vacuum at room temperature and the residue was purified by column chromatography to yield the compound 12 (4.2 g, 43%) as a colorless oil. MS (ESI) m/z [M+H]⁺ 506.35, [M+Na]⁺ 528.40.

Compound 13: To a stirred solution of FmocNH-PEG₆-COOH (1.15 g, 2.0 mmol) in a mixture of dry CH₂Cl₂ (20 mL) and DMF (20 mL) at room temperature under argon, the compound 12 (1.5 g, 2.2 mmol), EDCI (575 mg, 3.0 mmol) and HOBt (80 mg, 0.6 mmol) were added. The mixture was stirred at room temperature until full conversion was observed by HPLC. After the reaction was completed, the mixture was concentrated under vacuum. The crude reaction mixture was purified by silica gel chromatography to afford the product 13.

Compound 14: Compound 13 (1.25 g, 1.0 mmol) was dissolved in CH₂Cl₂ (10 mL) followed by addition of TFA (3.0 mL). The mixture was stirred at room temperature for 3 h. The solvent was removed under vacuum as much as possible at <35° C. The residue was purified by silica gel chromatography to afford the product 14.

Compound 15: To a stirred solution of compound 14 (865 mg, 0.8 mmol) in a mixture of dry CH₂Cl₂ (10 mL) and DMF (10 mL) at room temperature under argon, the compound 10 (2.4 g, 2.64 mmol), EDCI (863 mg, 4.5 mmol) and HOBt (108 mg, 0.8 mmol) were added. The mixture was stirred at room temperature until full conversion was observed by HPLC. After the reaction was completed, the mixture was concentrated under vacuum. The crude reaction mixture was purified by silica gel chromatography to afford the product 15.

Compound 16: Diethylamine (2.0 ml) is added to a solution of 15 (0.41 g, 0.11 mmol) in DMF (5 ml) and the reaction was allowed to proceed at room temperature for 2 h. After reaction, the reaction mixture was concentrated under vacuum and the residue was purified by preparative HPLC using Welch Ultimate XB-C18 column to yield the product 16.

Preparation of 30kmPEG-Lys(Mal)-3(Val-Cit-PAB-DEA-SN38) (Compound 22, FIG. 5)

Compound 18: H-Lys(boc)-OH (369 mg, 1.5 mmol) was added into anhydrous DMF (100 mL) followed by the addition of DIPEA (0.83 mL, 5.0 mmol), compound 30kmPEG-NHS (17) (15 g, 0.5 mmol) and anhydrous CH₂Cl₂ (150 mL). The mixture was stirred under argon at room temperature overnight. The insoluble materials were filtered off. The solvent was removed and the residue was recrystallized from CH₂Cl₂/methyl tert-butyl ether (45 mL/300 mL). The isolated solids were recrystallized again from MeCN/2-propanol (30 mL/450 mL). The product was dried at 40° C. over 4 h under vacuum to give the product 18 (13.6 g, 91%) as white powder.

Compound 19: The compound 18 (5.7 g, 0.19 mmol) was dissolved in anhydrous CH₂Cl₂ (57 mL) followed by the addition of TFA (29.5 mL). The mixture was stirred at room temperature for 1 h. Solvent was removed under vacuum as much as possible at <35° C. The residue was recrystallized from CH₂Cl₂/methyl tert-butyl ether (14.5 mL/115 mL) twice. The isolated product was dried under vacuum at 40° C. to yield the product 19 (4.7 g, 84%) as white powder.

Compound 21: To a stirred solution of compound 19 (5.5 g, 0.18 mmol) in anhydrous CH₂Cl₂ (55 mL) at 0° C., DIPEA (473 mg, 3.6 mmol) was added followed by 5-Maleimidovalericacid-NHS (20) (138 mg, 0.47 mmol). The mixture was stirred at 0° C. for 1.5 h, then allowed to warm up slowly from 0° C. to room temperature. The reaction was stirred under argon atmosphere overnight. Solvent was removed and the residue was recrystallized from CH₂Cl₂/methyl tert-butyl ether (13.8 mL/110 mL). The isolated solids were recrystallized again from MeCN/2-propanol (11 mL/165 mL). The residue was dried under vacuum to yield the product 21 (5.0 g, 90%) as white powder.

Compound 22: To a stirred solution of compound 21 (6.0 g, 0.2 mmol) in anhydrous CH₂Cl₂ (60 mL) at room temperature under argon, compound 16 (2.1 g, 0.6 mmol), EDCI (230 mg, 1.2 mmol) and HOBt (243 mg, 1.8 mmol) were added. The mixture was stirred at room temperature until full conversion was observed by HPLC. The solvent was removed and the residue was recrystallized from CH₂Cl₂/methyl tert-butyl ether (18 mL/120 mL). The isolated solids were recrystallized again from MeCN/2-propanol (12 mL/180 mL). The product was dried at 40° C. over 4 h under vacuum to give the product 22.

### Example 2. Preparation of 30kmPEG-Lys(Mal)-6(Val-Cit-PAB-DEA-SN38) (Compound 28, FIG. 6)

Compound 24: To a solution of compound FmocNH-PEG₆-COOH (1.15 g, 2.0 mmol) in dry CH₂Cl₂ (10 mL) at room temperature under argon, Di-tert-butyl 3,3′-azanediyldipropanoate (23) (0.64 mL, 2.2 mmol), EDCI (0.58 g, 3.0 mmol) and HOBt (54 mg, 0.4 mmol) were added. The mixture was stirred at room temperature until full conversion was observed by TLC. After the reaction was completed, the mixture was extracted with CH₂Cl₂ (30 mL×2). The organic layer was washed with brine (20 mL), dried over Na₂SO₄ and concentrated under vacuum. The crude reaction mixture was purified by silica gel chromatography to afford the product 24.

Compound 25: Compound 24 (0.39 g, 0.47 mmol) was dissolved in CH₂Cl₂ (4.0 mL) followed by the addition of TFA (2.0 mL). The mixture was stirred at room temperature for 3 h. The solvent was removed under vacuum as much as possible at <35° C. The residue was purified by silica gel chromatography to afford the product 25.

Compound 26: To a stirred solution of compound 25 (1.08 g, 1.5 mmol) in a mixture of dry CH₂Cl₂ (20 mL) and DMF (20 mL) at room temperature under argon, the compound 16 (11.7 g, 3.3 mmol), EDCI (767 mg, 4.0 mmol) and HOBt (108 mg, 0.8 mmol) were added. The mixture was stirred at room temperature until full conversion was observed by HPLC. After the reaction was completed, the mixture was concentrated under vacuum. The crude reaction mixture was purified by silica gel chromatography to afford the product 26.

Compound 27: Diethylamine (5.0 ml) was added to a solution of 26 (2.4 g, 0.31 mmol) in DMF (20 ml) and the reaction mixture was allowed to proceed at room temperature for 2 h. After reaction, the mixture was concentrated under vacuum and the residue was purified by preparative HPLC using Welch Ultimate XB-C18 column to yield the product 27.

Compound 28: To a stirred solution of compound 21 (6.0 g, 0.2 mmol) in anhydrous CH₂Cl₂ (60 mL) at room temperature under argon, compound 27 (4.5 g, 0.6 mmol), EDCI (230 mg, 1.2 mmol) and HOBt (243 mg, 1.8 mmol) were added. The mixture was stirred at room temperature until full conversion was observed by HPLC. The solvent was removed and the residue was recrystallized from CH₂Cl₂/methyl tert-butyl ether (18 mL/120 mL). The isolated solids were recrystallized again from MeCN/2-propanol (12 mL/180 mL). The product was dried at 40° C. over 4 h under vacuum to give the product 28.

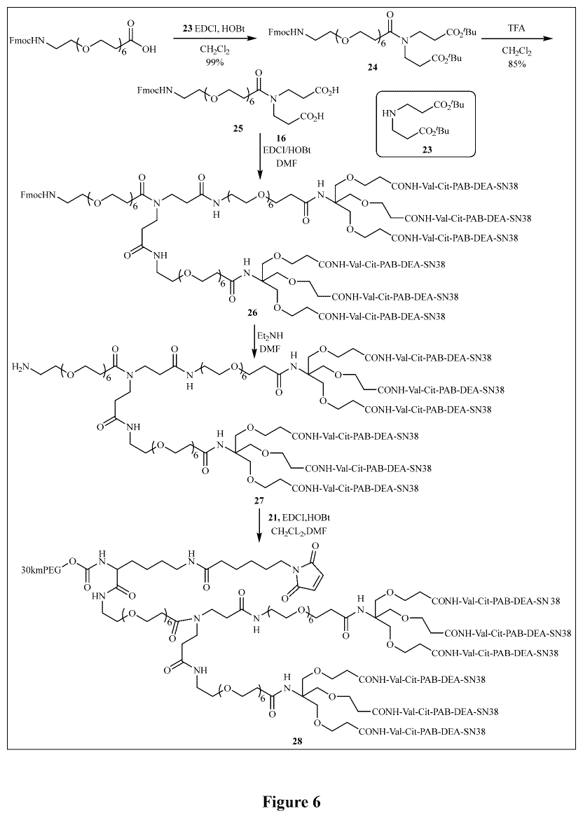

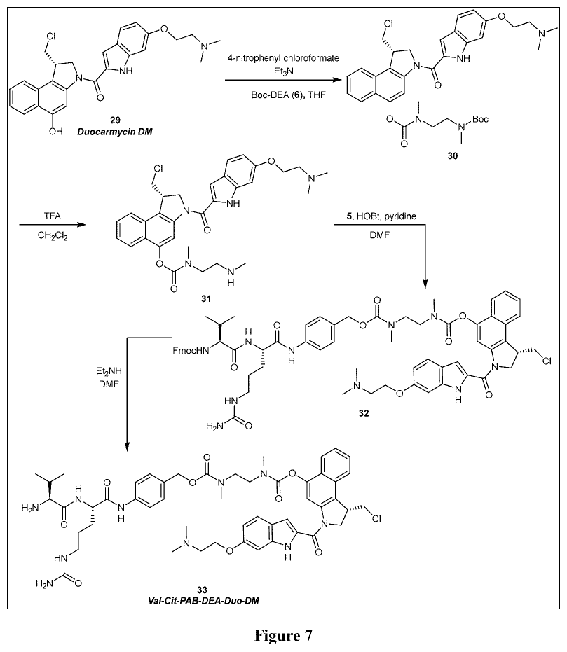

### Example 3. Preparation of 20kmPEG-Glu(Mal)-4(Val-Cit-PAB-DEA-Duo-DM) Preparation of Val-Cit-PAB-DEA-Duo-DM (Compound 33, FIG. 7)

BocDEA-Duo-DM (30): A solution of Duocarmycin DM (29) (4.6 g, 10 mmol) in anhydrous THF (100 mL) was cooled to 0° C. under a nitrogen atmosphere, after which 4-nitrophenyl chloroformate (2.7 g, 13.4 mmol) and Et₃N (7.0 mL, 50 mmol) were added. The mixture was stirred at 0° C. for 1.5 h. The compound Boc-DEA (6) (9.4 g, 50 mmol) was added, and the mixture was stirred for another 1 h. The reaction was slowly warmed to room temperature and then concentrated. The crude reaction mixture was purified by silica gel chromatography to afford the product 30.

DEA-Duo-DM (31): A solution of compound 30 (1.1 g, 1.62 mmol) in CH₂Cl₂ (10 mL) was cooled to 0° C., after which TFA (3 mL) was added. The mixture was stirred at 0° C. for 1 h and diluted with CH₂Cl₂ (10 mL). The diluted solution was concentrated under vacuum to yield crude 31.

Fmoc-Val-Cit-PAB-DEA-Duo-DM (32): The compound 31 (1.6 g, 2.8 mmol) and Fmoc-Val-Cit-PAB-PNP (5) (2.8 g, 3.6 mmol) were dissolved in DMF (20 m L). HOBt (0.75 g, 5.6 mmol) and pyridine (1.7 mL) were then added. The reaction mixture was stirred at room temperature for 24 h. After the reaction was completed, the reaction mixture was cooled to 0° C. Methyl tert-butyl ether (180 mL) was added. The resultant slurry was stirred for 3-5 h and filtered. The solids were washed and dried under vacuum. The crude product was purified by silica gel chromatography to afford the product 32.

Val-Cit-PAB-DEA-Duo-DM (33): Compound 32 (2.0 g, 1.7 mmol) was suspended in anhydrous DMF (40 mL) and the resulting suspension was stirred at room temperature until a homogeneous suspension was formed. Diethylamine (10 mL) was then added and the reaction mixture was stirred at room temperature for 3 h. After reaction was completed, methyl tert-butyl ether (100 mL) and ethyl acetate (50 mL) were then added over 60 min. The resulting mixture was stirred for 4 h at 0° C. Solids were filtered and dried under vacuum to yield compound 33.

Preparation of branched intermediate NH₂-PEG6-4(Val-Cit-PAB-DEA-Duo-DM) (Compound 40)

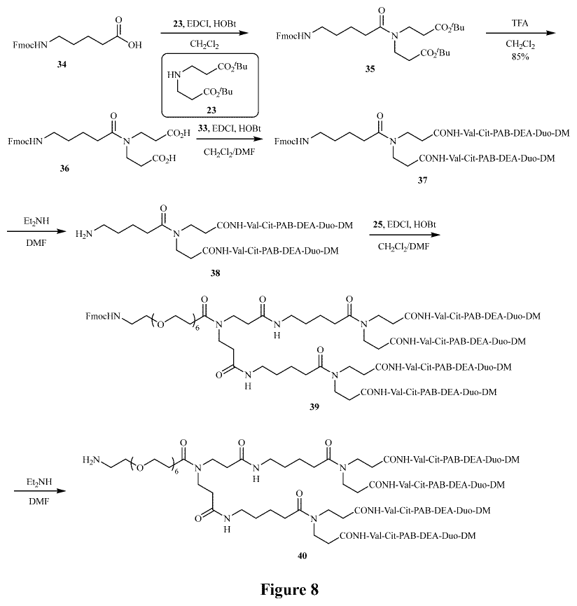

### Reaction Scheme a (FIG. 8)

Compound 35: To a solution of compound 34 (0.68 g, 2.0 mmol) in dry CH₂Cl₂ (10 mL) at room temperature under argon, Di-tert-butyl 3,3′-azanediyldipropanoate (23) (0.64 mL, 2.2 mmol), EDCI (0.58 g, 3.0 mmol) and HOBt (54 mg, 0.4 mmol) are added. The mixture is stirred at room temperature until full conversion is observed by TLC. After the reaction is completed, the mixture is extracted with CH₂Cl₂ (30 mL×2), and the combined organic layer is washed with brine (20 mL), dried over Na₂SO₄ and concentrated under vacuum. The crude product is purified by silica gel chromatography to afford the product 35.

Compound 36: Compound 35 (0.5 g, 0.84 mmol) is dissolved in CH₂Cl₂ (6.0 mL) followed by addition of TFA (3.0 mL). The mixture is stirred at room temperature for 3 h. The solvent is removed under vacuum as much as possible at <35° C. The residue is purified by silica gel chromatography to afford the product 36.

Compound 37: To a solution of compound 36 (964 mg, 2.0 mmol) in a mixture of dry CH₂Cl₂ (8 mL) and DMF (8 mL) at room temperature under argon, Val-Cit-PAB-DEA-Duo-DM (33) (4.3 g, 4.4 mmol), EDCI (575 mg, 3.0 mmol) and HOBt (135 mg, 1.0 mmol) are added. The mixture is stirred at room temperature until full conversion is observed by HPLC. After the reaction is completed, the mixture is concentrated under vacuum. The crude reaction mixture is purified by silica gel chromatography to afford the product 37.

Compound 38: Diethylamine (2.0 ml) is added to a solution of 37 (0.5 g) in DMF (5 ml) and the reaction mixture is allowed to proceed at room temperature for 2 h. After reaction, the reaction mixture is concentrated under vacuum and the residue is purified by preparative HPLC using Welch Ultimate XB-C18 column to yield the product 38.

Compound 39: To a stirred solution of compound 25 (546 mg, 0.76 mmol) in a mixture of dry CH₂Cl₂ (20 mL) and DMF (20 mL) at room temperature under argon, the compound 38 (3.7 g, 1.7 mmol), EDCI (430 mg, 2.3 mmol) and HOBt (40 mg, 0.3 mmol) are added. The mixture is stirred at room temperature until full conversion is observed by HPLC. After the reaction is completed, the mixture is concentrated under vacuum. The crude reaction mixture is purified by silica gel chromatography to afford the product 39.

Compound 40: Diethylamine (2.0 ml) is added to a solution of 39 (0.51 g, 0.1 mmol) in DMF (5 ml) and the reaction mixture is allowed to proceed at room temperature for 2 h. After reaction, the reaction mixture wis concentrated under vacuum and the residue is purified by preparative HPLC using Welch Ultimate XB-C18 column to yield the product 40.

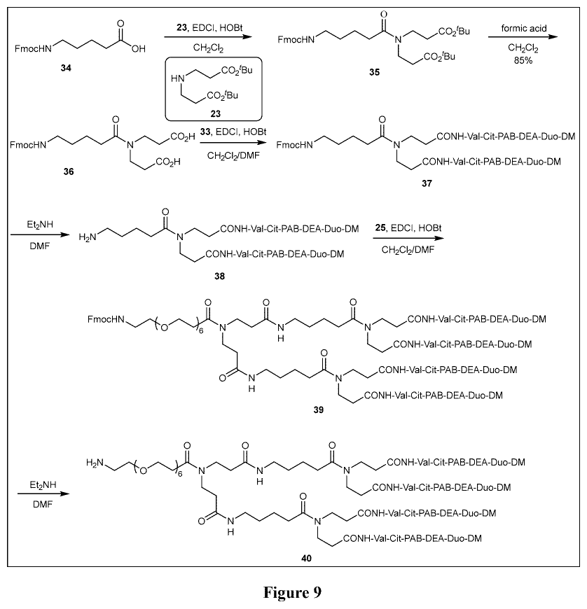

### Reaction Scheme B (FIG. 9)

Compound 35: To a solution of compound 34 (0.62 g, 2.0 mmol) in dry CH₂Cl₂ (15 mL) at room temperature under argon, Di-tert-butyl 3,3′-azanediyldipropanoate (23) (0.62 mL, 2.2 mmol), EDCI (0.58 g, 3.0 mmol) and HOBt (54 mg, 0.4 mmol) were added. The mixture was stirred at room temperature until full conversion was observed by TLC. After the reaction was completed, the mixture was extracted with CH₂Cl₂ (30 mL×2), and the combined organic layer was washed with brine (20 mL), dried over Na₂SO₄ and concentrated under vacuum. The crude product was purified by silica gel chromatography to afford the product 35 (1.1 g, 96%) as colorless oil. HRMS (ESI) calcd for C₃₂H₄₃N₂O₇ [M+H]⁺ 567.3070, found 567.3062.

Compound 36: Compound 35 (0.5 g, 0.84 mmol) was dissolved in formic acid (3.0 mL). The mixture was stirred at room temperature for 16 h. The solvent was removed under vacuum as much as possible at <35° C. The residue is purified by silica gel chromatography to afford the product 36 (1.5 g, 94%) as colorless oil. HRMS (ESI) calcd for C₂₄H₂₇N₂O₇ [M+H]³⁰ 455.1818, found 455.1824.

Compound 37: To a solution of compound 36 (964 mg, 2.0 mmol) in a mixture of dry CH₂Cl₂ (8 mL) and DMF (8 mL) at room temperature under argon, Val-Cit-PAB-DEA-Duo-DM (33) (4.3 g, 4.4 mmol), EDCI (575 mg, 3.0 mmol) and HOBt (135 mg, 1.0 mmol) were added. The mixture was stirred at room temperature until full conversion was confirmed by HPLC. After the reaction was completed, the mixture was concentrated under vacuum. The crude product was purified by silica gel chromatography to afford the product 37.

Compound 38: Diethylamine (2.0 mL) was added to a solution of compound 37 (0.5 g) in DMF (5.0 mL) and the reaction was allowed to proceed at room temperature for 2 h. After reaction, the reaction mixture was concentrated under vacuum and the residue was purified by preparative HPLC using Welch Ultimate XB-C18 column to yield the product 38.

Compound 39: To a stirred solution of compound 25 (546 mg, 0.76 mmol) in dry CH₂Cl₂ (20 mL) and DMF (20 mL) at room temperature under argon, the compound 38 (3.7 g, 1.7 mmol), EDCI (430 mg, 2.3 mmol) and HOBt (40 mg, 0.3 mmol) were added. The mixture was stirred at room temperature until full conversion was confirmed by HPLC. After the reaction was completed, the mixture was concentrated under vacuum. The crude product was purified by silica gel chromatography to afford the product 39.

Compound 40: Diethylamine (2.0 ml) was added to a solution of 39 (0.51 g, 0.1 mmol) in DMF (5 mL) and the reaction was allowed to proceed at room temperature for 2 h. After reaction, the reaction mixture was concentrated under vacuum and the residue was purified by preparative HPLC using Welch Ultimate XB-C18 column to yield the product 40.

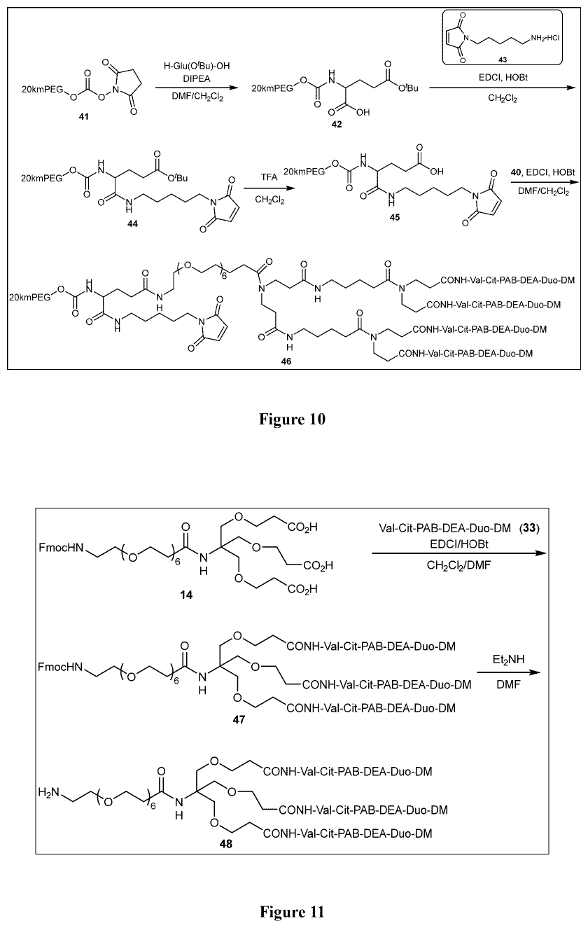

Preparation of 20kmPEG-Glu(Mal)-4(Val-Cit-PAB-DEA-Duo-DM) (Compound 46, FIG. 10)

Compound 42: H-Glu(OtBu)-OH (305 mg, 1.5 mmol) was added into anhydrous DMF (67 mL) followed by addition of DIPEA (0.83 mL, 5.0 mmol), compound 20kmPEG-NHS (41) (10 g, 0.5 mmol) and anhydrous CH₂Cl₂ (100 mL). The mixture was stirred under argon at room temperature overnight. The solvent was removed and the residue was recrystallized from CH₂Cl₂/methyl tert-butyl ether (30 mL/200 mL). The isolated solids were recrystallized again from MeCN/2-propanol (20 mL/300 mL). The product was dried at 40° C. over 4 h under vacuum to give the product 42 as white powder.

Compound 44: To a stirred solution of compound 42 (2.0 g, 0.1 mmol) in anhydrous CH₂Cl₂ (20 mL) at room temperature under argon, compound 43 (66 mg, 0.3 mmol), EDCI (115 mg, 0.6 mmol) and HOBt (122 mg, 0.9 mmol) were added. The mixture was stirred at room temperature until full conversion was confirmed by HPLC. The solvent was removed and the residue was recrystallized from CH₂Cl₂/methyl tert-butyl ether (5 mL/40 mL). The isolated solids were recrystallized again from MeCN/2-propanol (4 mL/60 mL). The product was dried at 40° C. over 4 h under vacuum to give the product 44 as white powder.

Compound 45: The compound 44 (5.8 g, 0.29 mmol) was dissolved in anhydrous CH₂Cl₂ (58 mL). TFA (29 mL) was added. The mixture was stirred at room temperature for 1 h. Solvent was removed under vacuum as much as possible at <35° C. The residue was recrystallized from CH₂Cl₂/methyl tert-butyl ether (14.5 mL/115 mL) twice. The isolated product was dried under vacuum at 40° C. to yield the product 45 as white powder.

Compound 46: To a stirred solution of compound 45 (6.0 g, 0.3 mmol) in anhydrous CH₂Cl₂ (60 mL) at room temperature under argon, the compound 40 (4.4 g, 0.9 mmol), EDCI (345 mg, 1.8 mmol) and HOBt (365 mg, 2.7 mmol) were added. The mixture was stirred at room temperature until full conversion was confirmed by HPLC. The solvent was removed and the residue was recrystallized from CH₂Cl₂/methyl tert-butyl ether (18 mL/120 mL). The isolated solids were recrystallized again from MeCN/2-propanol (12 mL/180 mL). The product was dried at 40° C. over 4 h under vacuum to give the compound 46.

### Example 4. Preparation of Mal-20kPEG-3(Val-Cit-PAB-DEA-Duo-DM)

Preparation of Branched Intermediate NH₂-PEG₆-3(Val-Cit-PAB-DEA-Duo-DM) (Compound 48, FIG. 11)

Compound 47: To a stirred solution of compound 14 (865 mg, 0.8 mmol) in a mixture of dry CH₂Cl₂ (10 mL) and DMF (10 mL) at room temperature under argon, compound 33 (2.6 g, 2.64 mmol), EDCI (863 mg, 4.5 mmol) and HOBt (108 mg, 0.8 mmol) were added. The mixture was stirred at room temperature until full conversion was confirmed by HPLC. After the reaction was completed, the mixture was concentrated under vacuum. The crude reaction mixture was purified by silica gel chromatography to afford the product 47.

Compound 48: Diethylamine (2.0 ml) was added to a solution of compound 47 (0.48 g, 0.12 mmol) in DMF (5.0 mL) and the reaction was allowed to proceed at room temperature for 2 h. After reaction, the reaction mixture was concentrated under vacuum and the residue was purified by preparative HPLC using Welch Ultimate XB-C18 column to yield the product 48.

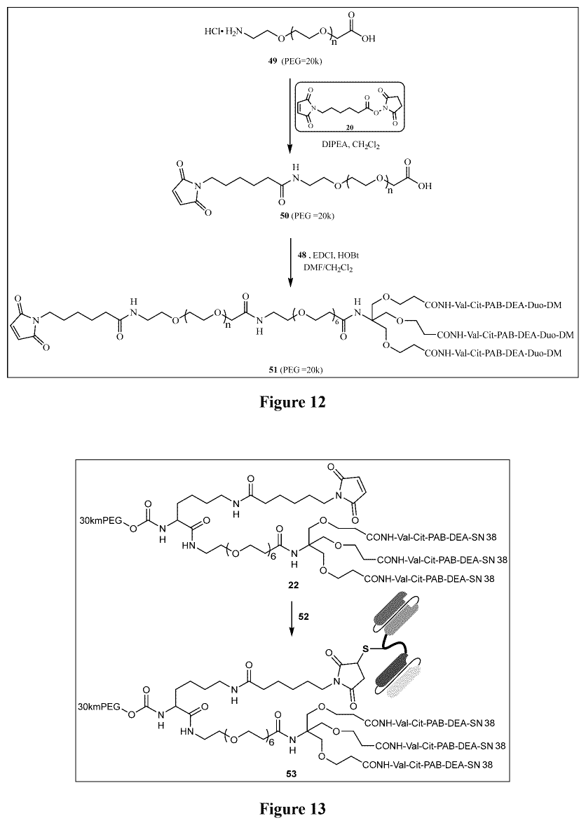

### Preparation of Mal-20kPEG-3(Val-Cit-PAB-DEA-Duo-DM) (Compound 51, FIG. 12)

Compound 50: amine-PEG20k-CO₂H (49) (1.0 g, 0.05 mmol) was dissolved in anhydrous CH₂Cl₂ (10 mL) at 0° C. DIPEA (83 μL, 0.5 mmol) was added followed by addition of compound 20 (46 mg, 0.15 mmol). The mixture was stirred at 0° C. for 1.5 h. After reaction, the solution was allowed to warm up slowly from 0° C. to room temperature and then stirred under argon atmosphere overnight. Solvent was removed and the residue was recrystallized from CH₂Cl₂/methyl tert-butyl ether (2.5 mL/20 mL). The isolated solids were recrystallized again from MeCN/2-propanol (2 mL/30 mL). The residue was dried under vacuum to yield the product 50.

Compound 51: To a stirred solution of compound 50 (6.0 g, 0.2 mmol) in anhydrous CH₂Cl₂ (60 mL) at room temperature under argon, compound 48 (2.1 g, 0.6 mmol), EDCI (230 mg, 1.2 mmol) and HOBt (243 mg, 1.8 mmol) were added. The mixture was stirred at room temperature until full conversion was confirmed by HPLC. The solvent was removed and the residue was recrystallized from CH₂Cl₂/methyl tert-butyl ether (18 mL/120 mL). The isolated solids were recrystallized again from MeCN/2-propanol (12 mL/180 mL). The product was dried at 40° C. over 4 h under vacuum to give the product 51.

### Example 5. Preparation of SCAPDL1xSCACD47 (Compound 52)

Bispecific single chain antibody (SCA) of anti-PDL1 and anti-CD47 (SCAPDL1xSCACD47) was prepared via recombinant DNA technology in mammalian cells (e.g., CHO using Easy Select™) or yeast (e.g., Pichia pastori Expression Kit containing a pPICZ vector). To facilitate the subsequent conjugation, a site specific conjugation functional group thiol was inserted through recombinant DNA technology into the linker between two PDL1 and CD47 SCAs. DNA Sequences of SCAPDL1xSCACD47 corresponding to amino acid sequence below (SEQ ID No. 1) were synthesized and cloned into the expression vectors and transformed in the host cells. After cell expression, supernatant of culture media of host cells expressing SCAPDL1xSCACD47 was collected after centrifugation and loaded to a Ni-charged column (2.6 cm×13 cm) (Cat #AA207311, BestChrome, Shanghai, China) pre-equilibrated with 50 mM sodium phosphate, 100 mM of NaCl, pH 7.0. The protein was eluted off with a buffer of 50 mM sodium phosphate, 250 mM imidazole, 100 mM ofNaCl, pH 7.0 and fractionated in 15 mL tubes. Captured protein was further purified with a CaptoL column (Cat #17-5478-02, GE Healthcare, NJ) (1.6 cm×8 cm), which was pre-equilibrated with 50 mM of sodium phosphate and 100 mM of NaCl (pH 7.0). The protein was eluted with 75 mM of acetic acid (pH 3.0) to give isolated product 52.

Amino acid Sequence of SCAPDL1xSCACD47
(SEQ ID No. 1):
DIVMTQSPLSLPVTPGEPASISCRSSQSIVYSNGNTYLGWYLQKP
GQSPQLLIYKVSNRFSGVPDRFSGSGSGTDFTLKISRVEAEDVGV
YYCFQGSHVPYTFGQGTKLEIKGGSGGSGGSGGSGGQVQLVQSGA
EVKKPGASVKVSCKASGYTFTNYNMHWVRQAPGQRLEWMGTIYPG
NDDTSYNQKFKDRVTITADTSASTAYMELSSLRSEDTAVYYCARG
GYRAMDYWGQGTLVTVSSGCGGSSGGSDIQMTQSPSSLSASVGDR
VTITCRASQDVSTAVAWYQQKPGKAPKLLIYSASFLYSGVPSRFS
GSGSGTDFTLTISSLQPEDFATYYCQQYLYHPATFGQGTKVEIKG
GGGSGGGGSGGGGSEVQLVESGGGLVQPGGSLRLSCAASGFTFSD
SWIHWVRQAPGKGLEWVAWISPYGGSTYYADSVKGRFTISADTSK
NTAYLQMNSLRAEDTAVYYCARRHWPGGFDYWGQGTLVTHHHHHH.

### Example 6. Preparation of 30kmPEG(SCAPDL1xSCACD47)-3(Val-Cit-PAB-DEA-SN38) (Compound 53, FIG. 13)

A solution of protein 52 is adjusted to pH 6.8 with a pH 4.12 stock solution of 500 mM sodium phosphate, followed by reduction with 3.5 mM of TCEP-HCl at room temperature for 30 min. The reduced protein is adjusted to 5 mg/mL. Pegylation of SCAPDL1xSCACD47 is conducted at room temperature for 3 hours at pH 6.8 with 5 to 10 equivalent of compound 22 [30kmPEG-Lys(Mal)-3(Val-Cit-PAB-DEA-SN38)]. The reaction is quenched with 10 mM of L-cystine at room temperature for 10 min. Final product 53 [30kmPEG(SCAPDL1xSCACD47)-3(Val-Cit-PAB-DEA-SN38)] is purified with a cation exchange chromatography column (CM Fast Flow) at pH 6.5 in 20 mM of phosphate buffer. The target compound 53 is confirmed by SEC-HPLC and cell-based activity assay.

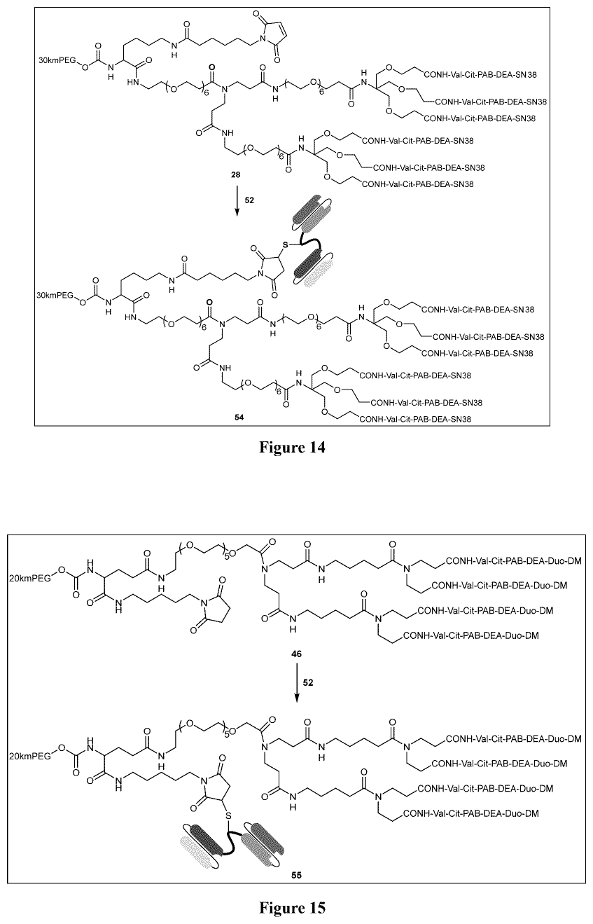

### Example 7. Preparation of 30kmPEG-(SCAPDL1xSCACD47)-6(Val-Cit-PAB-DEA-SN38) (Compound 54, FIG. 14)

Compound 54 is made by conjugation of compound 28 [30kmPEG-Lys(Mal)-6(Val-Cit-PAB-DEA-SN38)] with protein 52 with similar procedures as for preparation of compound 53.

### Example 8. Preparation of 20kmPEG-(SCAPDL1xSCACD47)-4(Val-Cit-PAB-DEA-Duo-DM) (Compound 55, FIG. 15)

Compound 55 is made by conjugation of compound 46 [20kmPEG-Glu(Mal)-4(Val-Cit-PAB-DEA-Duo-DM)] with protein 52 with similar procedures as for preparation of compound 53.

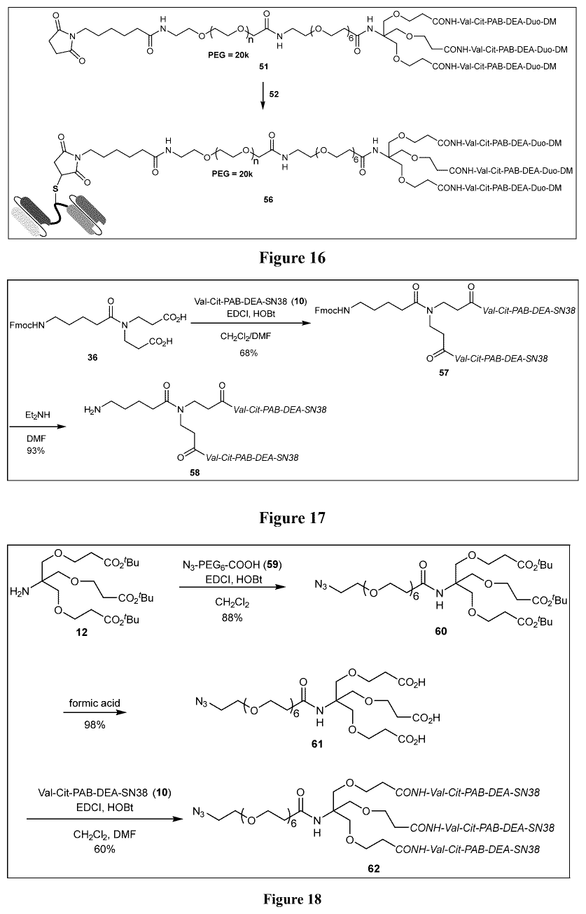

### Example 9. Preparation of SCAPDL1xSCACD47-20kPEG-3(Val-Cit-PAB-DEA-Duo-DM) (Compound 56, FIG. 16)

Compound 56 is made by conjugation of compound 51 [Mal-20kPEG-3(Val-Cit-PAB-DEA-Duo-DM)] with protein 52 with similar procedures as for preparation of compound 53.

### Example 10. Preparation of Branched Intermediate NH₂-2(Val-Cit-PAB-DEA-SN38) (Compound 58, FIG. 17)

Compound 57: To a stirred solution of compound 36 (0.75 g, 1.55 mmol) in dry DMF (30 mL) at room temperature under argon, Val-Cit-PAB-DEA-SN38 (10) (3.1 g, 3.42 mmol), EDCI (0.89 g, 4.66 mmol) and HOBt (0.21 g, 1.55 mmol) were added. The mixture was stirred at room temperature until full conversion was observed by TLC. After the reaction was completed, the mixture was concentrated in vacuum. The reaction mixture was cooled to 0° C. and was added methyl tert-butyl ether (90 mL). The resultant slurry was stirred for 1 h, filtered, washed and dried under vacuum. The crude product was purified through chromatography on silica gel to afford the compound 57 (2.4 g, 68%) as white solid. MS (ESI) m/z [M+2H]²⁺ 1135.82.

Compound 58: Diethylamine (4.0 mL) was added to a solution of compound 57 (1.2 g) in DMF (12 mL), then the reaction was allowed to proceed at room temperature for 40 min. The reaction mixture was concentrated in vacuum and the resulting residue was slurred in methyl tert-butyl ether (90 mL) for 2 h. Solids were filtered, washed and dried under vacuum to yield compound 58 (1.0 g, 93%) as pale yellow powder. MS (ESI) m/z [M+2H]²⁺ 1024.67, [M+2Na]²⁺ 1046.63.

### Example 11. Preparation of Branched Intermediate N₃-PEG₆-3(Val-Cit-PAB-DEA-SN38) (Compound 62, FIG. 18)

Compound 60: To a solution of compound 12 (1.5 g, 2.9 mmol) in dry CH₂Cl₂ (20 mL) at room temperature under argon, compound N₃-PEG₆-CO₂H (59) (1.0 g, 2.64 mmol), EDCI (0.76 g, 4.0 mmol) and HOBt (110 mg, 0.8 mmol) were added. The mixture was stirred at room temperature until full conversion was confirmed by TLC. After the reaction was completed, the mixture was extracted with CH₂Cl₂ (30 mL×2), and the organic layer was washed with brine (20 mL), dried over Na₂SO₄, filtered and concentrated in vacuum. The crude reaction mixture was purified on silica gel column to afford the product 60 (2.0 g, 88%) as colorless oil. HRMS (ESI) calcd for C₃₂H₄₃N₂O₇ [M+H]⁺ 567.3070, found 567.3062. MS (ESI) m/z [M+H]⁺ 867.55, [M+Na]⁺ 889.40.

Compound 61: Compound 60 (2.0 g, 3.1 mmol) was dissolved in formic acid (30 mL), the mixture was stirred at room temperature for 16 h. The solvent was removed under vacuum as much as possible at <35° C. The residue was purified on silica gel column to afford the product 61 (1.5 g, 98%) as colorless oil. MS (ESI) m/z [M+H]⁺ 699.24, [M+Na]⁺ 721.34.

Compound 62: To a stirred solution of compound 61 (0.6 g, 0.86 mmol) in dry DMF (25 mL) at room temperature under argon, Val-Cit-PAB-DEA-SN38 (10) (2.6 g, 2.8 mmol), EDCI (0.74 g, 3.86 mmol) and HOBt (0.12 g, 0.86 mmol) were added. The mixture was stirred at room temperature until full conversion was confirmed by HPLC. After the reaction was completed, the mixture was concentrated under vacuum. The reaction mixture was cooled to 0° C. and was added to methyl tert-butyl ether (120 mL). The resultant slurry was stirred for 1 h, filtered, washed and dried under vacuum. The crude product was purified on silica gel column to afford the compound 62 (1.7 g, 60%) as white solid. MS (ESI) m/z [M+3H]³⁺ 1127.53, [M+3Na]³⁺ 1149.58.

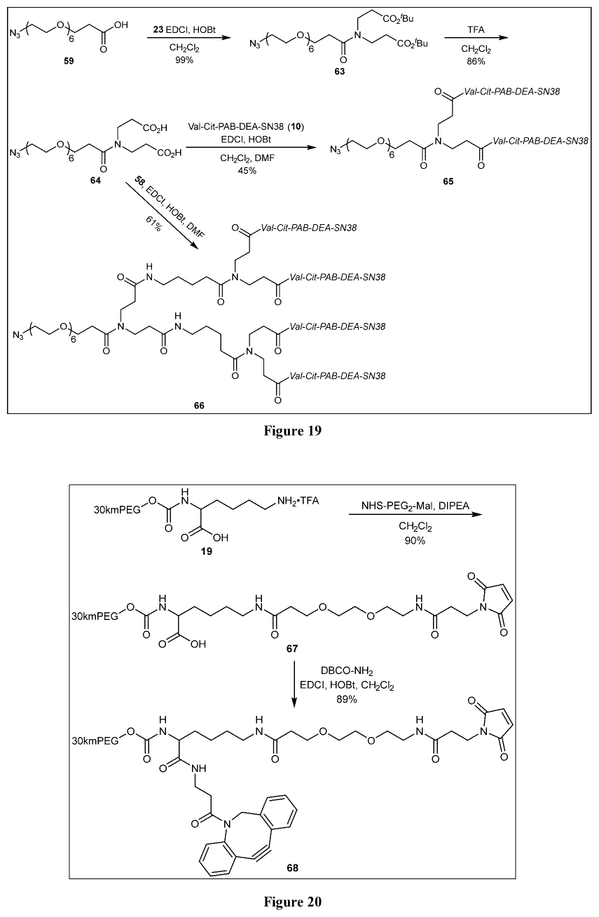

### Example 12. Preparation of Branched Compound 65 [N₃-PEG₆-2(Val-Cit-PAB-DEA-SN38)] and 66 [N₃-PEG₆-4(Val-Cit-PAB-DEA-SN38)](FIG. 19)

Compound 63: To a solution of compound 59 (0.76 g, 2.0 mmol) in dry CH₂Cl₂ (10 mL) at room temperature under argon, Di-tert-butyl 3,3′-azanediyldipropanoate (23) (0.64 mL, 2.2 mmol), EDCI (0.58 g, 3.0 mmol) and HOBt (54 mg, 0.4 mmol) were added. The mixture was stirred at room temperature until full conversion was confirmed by TLC. After the reaction was completed, the mixture was extracted with CH₂Cl₂ (30 mL×2), and the organic layer was washed with brine (20 mL), dried over Na₂SO₄, filtered and concentrated under vacuum. The crude reaction mixture was purified on silica gel column to afford the product 63 (1.2 g, 99%) as colorless oil. HRMS (ESI) calcd for C₂₉H₅₅N₄O₁₁ [M+H]⁺ 635.3867, found 635.3860.

Compound 64: Compound 63 (0.87 g, 1.37 mmol) was dissolved in CH₂Cl₂ (10 mL) followed by addition of TFA (4.0 mL). The mixture was stirred at room temperature for 2 h. The solvent was removed under vacuum as much as possible at <35° C. The residue was purified on silica gel column to afford the product 64 (0.62 g, 86%) as colorless oil. HRMS (ESI) calcd for C₂₁H₃₉N₄O₁₁ [M+H]⁺ 523.2615, found 523.2607.

Compound 65: To a stirred solution of compound 64 (0.6 g, 1.15 mmol) in dry DMF (20 mL) at room temperature under argon, Val-Cit-PAB-DEA-SN38 (10) (2.3 g, 2.5 mmol), EDCI (0.66 g, 3.5 mmol) and HOBt (90 mg, 0.7 mmol) were added. The mixture was stirred at room temperature until full conversion was confirmed by HPLC. After the reaction was completed, the mixture was concentrated under vacuum. The reaction mixture was cooled to 0° C. and was added to methyl tert-butyl ether (100 mL). The resultant slurry was stirred for 1 h, filtered, washed and dried under vacuum. The crude product was purified on silica gel column to afford the compound 65 (1.2 g, 45%) as white solid. MS (ESI) m/z [M+2H]²⁺ 1155.92, [M+2Na]²⁺ 1177.87.

Compound 66: To a stirred solution of compound 64 (43 mg, 0.08 mmol) in a mixture of dry CH₂Cl₂ (2 mL) and DMF (2 mL) at room temperature under argon, compound 58 (0.42 g, 0.21 mmol), EDCI (47 mg, 0.25 mmol) and HOBt (5.4 mg, 0.04 mmol) were added. The mixture was stirred at room temperature until full conversion was confirmed by HPLC. After the reaction was completed, the mixture was concentrated in vacuum. The crude reaction mixture was purified through preparative HPLC using Welch Ultimate XB-C18 column (eluents: A=0.1% TFA in water, B=MeCN) to yield the product 66 (230 mg, 61%) as pale yellow solid. MS (ESI) m/z [M+3H]³⁺ 1528.62, [M+4H]⁴⁺ 1146.87.

### Example 13. Preparation of 30kmPEG-Lys(PEG₂-Mal)-DBCO (Compound 68, FIG. 20)

Compound 67: To a stirred solution of compound 19 (5.5 g, 0.18 mmol) in anhydrous CH₂Cl₂ (55 mL) at 0° C., DIPEA (473 mg, 3.6 mmol) was added followed by addition of NHS-PEG2-Mal (0.2 g, 0.47 mmol). The mixture was stirred at 0° C. for 1.5 h, then the solution was allowed to warm up slowly from 0° C. to room temperature and then stirred under argon atmosphere overnight. Solvent was removed and the residue was recrystallized from CH₂Cl₂/methyl tert-butyl ether (13.8 mL/110 mL). The isolated solids were recrystallized again from MeCN/2-propanol (11 mL/165 mL). The residue was dried under vacuum to yield the product 67 (5.0 g, 90%) as white powder.

Compound 68: To a stirred solution of compound 67 (3.0 g, 0.1 mmol) in anhydrous CH₂Cl₂ (30 mL) at room temperature under argon, DBCO-NH₂ (83 mg, 0.3 mmol), EDCI (115 mg, 0.6 mmol) and HOBt (122 mg, 0.9 mmol) were added. The mixture was stirred at room temperature until full conversion was confirmed by HPLC. The solvent was removed and the residue was recrystallized from CH₂Cl₂/methyl tert-butyl ether (5 mL/40 mL). The isolated solids were recrystallized again from MeCN/2-propanol (4 mL/60 mL). The product was dried at 40° C. over 4 h under vacuum to give the product 68 (2.7 g, 89%) as white powder.

### Example 14. Preparation of 30kmPEG-Lys(PEG₂-Mal)-2(Val-Cit-PAB-DEA-SN38) (Compound 69, FIG. 21)

Compound 69: To a stirred solution of compound 67 (0.9 g, 0.03 mmol) in a mixture of DMF/CH₂Cl₂ (5 mL/5 mL) at room temperature under argon, compound 58 (0.16 g, 0.08 mmol), EDCI (35 mg, 0.18 mmol) and HOBt (37 mg, 0.27 mmol) were added. The mixture was stirred at room temperature until full conversion was confirmed by HPLC. The solvent was removed and the residue was recrystallized from CH₂Cl₂/methyl tert-butyl ether (2.5 mL/20 mL). The isolated solids were recrystallized again from MeCN/2-propanol (2 mL/30 mL). The product was dried at 40° C. over 4 h under vacuum to give the product 69 (0.7 g, 80%) as white powder. MS (MALDI-TOF) m z 32065.3 Da.

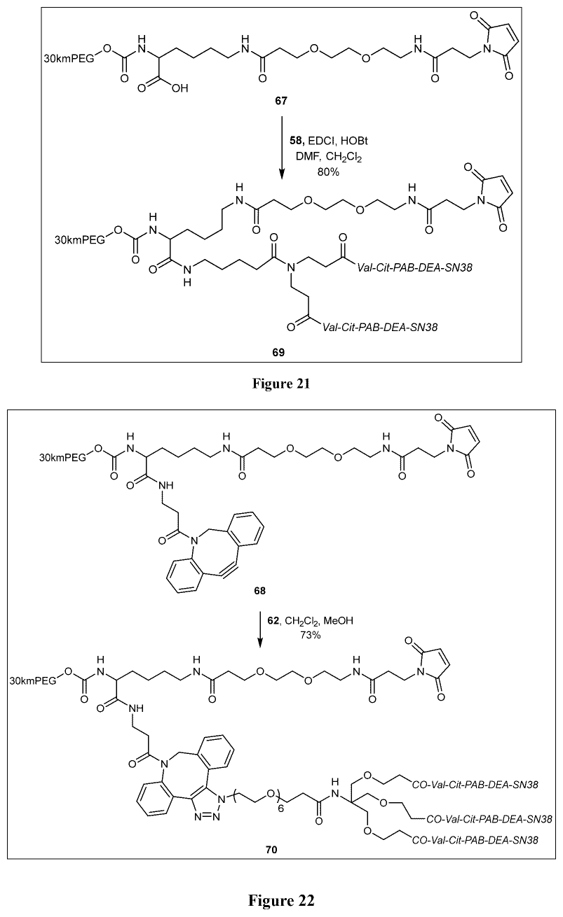

### Example 15. Preparation of 30kmPEG-Lys(PEG₂-Mal)-3(Val-Cit-PAB-DEA-SN38) (Compound 70, FIG. 22)

Compound 70: To a stirred solution of compound 68 (1.5 g, 0.05 mmol) in anhydrous CH₂Cl₂ (10 mL) and MeOH (10 mL), compound 62 (0.5 g, 0.15 mmol) was added. The mixture was stirred at room temperature overnight until full conversion was confirmed by HPLC. Solvent was removed and the residue was recrystallized from MeCN/2-propanol (3 mL/45 mL) twice.. The residue was dried under vacuum to yield the product 70 (1.1 g, 73%) as white powder.

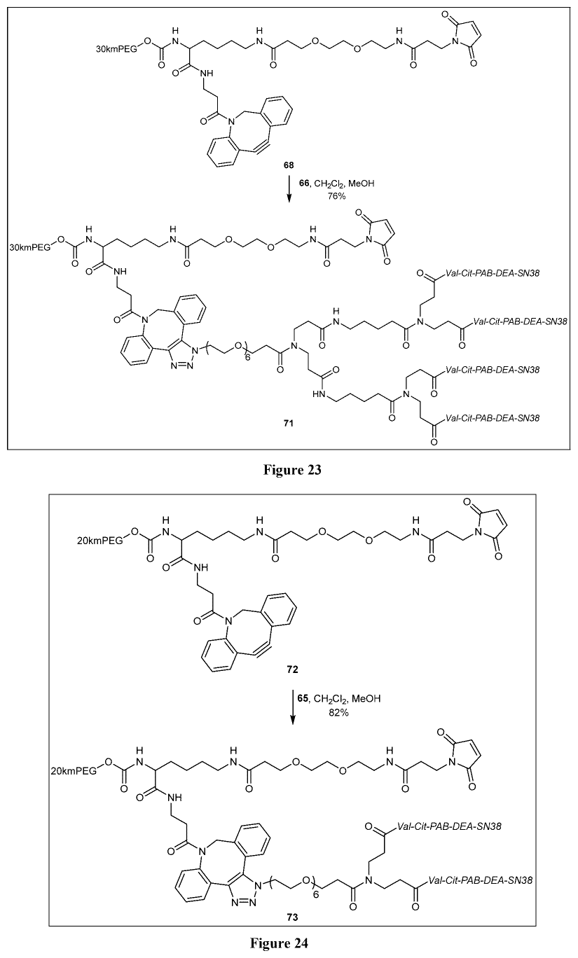

### Example 16. Preparation of 30kmPEG-Lys(PEG₂-Mal)-4(Val-Cit-PAB-DEA-SN38) (Compound 71, FIG. 23)

Compound 71: To a stirred solution of compound 68 (1.2 g, 0.04 mmol) in anhydrous CH₂Cl₂ (8 mL) and MeOH (8 mL), compound 66 (0.37 g, 0.08 mmol) was added. The mixture was stirred at room temperature overnight until full conversion was confirmed by HPLC. Solvent was removed and the residue was recrystallized from MeCN/2-propanol (3 mL/45 mL) twice. The residue was dried under vacuum to yield the product 71 (0.91 g, 73%) as white powder.

### Example 17. Preparation of 20kmPEG-Lys(PEG₂-Mal)-2(Val-Cit-PAB-DEA-SN38) (Compound 73, FIG. 24)

Compound 73: To a stirred solution of compound 72 (2.0 g, 0.1 mmol) (to make compound 72, refer to the procedure for preparation of compound 68) in anhydrous CH₂Cl₂ (20 mL) at room temperature under argon, compound 65 was added. The mixture was stirred at room temperature until full conversion was confirmed by HPLC. The solvent was removed and the residue was recrystallized from MeCN/2-propanol (4 mL/60 mL) twice. The product was dried at 40° C. over 4 h under vacuum to give the product 73 (1.6 g, 82%) as white powder.

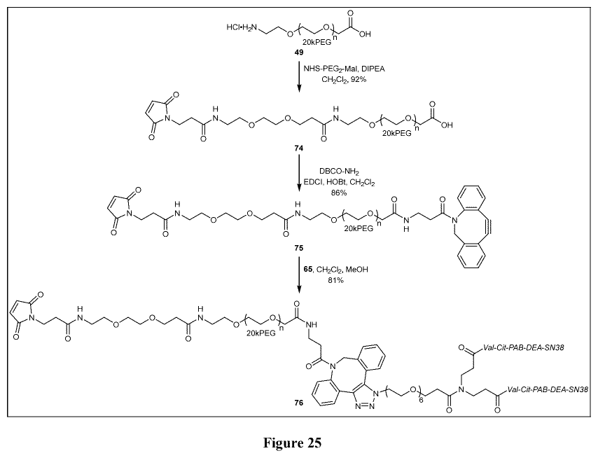

### Example 18. Preparation of Mal-PEG₂-20kPEG-2(Val-Cit-PAB-DEA-SN38) (Compound 76, FIG. 25)

Compound 74: To a stirred solution of amine-PEG20k-CO₂H (49) (1.0 g, 0.05 mmol) in anhydrous CH₂Cl₂ (10 mL) at 0° C., DIPEA (83 μL, 0.5 mmol) was added followed by addition of NHS-PEG₂-Mal (64 mg, 0.15 mmol). The mixture was stirred at 0° C. for 1.5 h, then the solution was allowed to warm up slowly from 0° C. to room temperature and then stirred under argon atmosphere overnight. Solvent was removed and the residue was recrystallized from CH₂Cl₂/methyl tert-butyl ether (2.5 mL/20 mL). The isolated solids were recrystallized again from MeCN/2-propanol (2 mL/30 mL). The residue was dried under vacuum to yield the product 74 (0.92 g, 92%) as white powder.

Compound 75: To a stirred solution of compound 74 (0.9 g, 0.045 mmol) in anhydrous CH₂Cl₂ (9 mL) at room temperature under argon, DBCO-NH₂ (37 mg, 0.14 mmol), EDCI (52 mg, 0.27 mmol) and HOBt (55 mg, 0.41 mmol) were added. The mixture was stirred at room temperature until full conversion was confirmed by HPLC. The solvent was removed and the residue was recrystallized from CH₂Cl₂/methyl tert-butyl ether (2.5 mL/20 mL). The isolated solids were recrystallized again from MeCN/2-propanol (2 mL/30 mL). The product was dried at 40° C. over 4 h under vacuum to give the product 75 (0.77 g, 86%) as white powder.

Compound 76: To a stirred solution of compound 75 (0.7 g, 0.035 mmol) in anhydrous CH₂Cl₂ (8 mL) and MeOH (8 mL), compound 65 (0.17 g, 0.08 mmol) was added. The mixture was stirred at room temperature overnight until full conversion was observed by HPLC. Solvent was removed and the residue was recrystallized from MeCN/2-propanol (2 mL/30 mL) twice. The residue was dried under vacuum to yield the product 76 (0.57 g, 81%) as white powder.

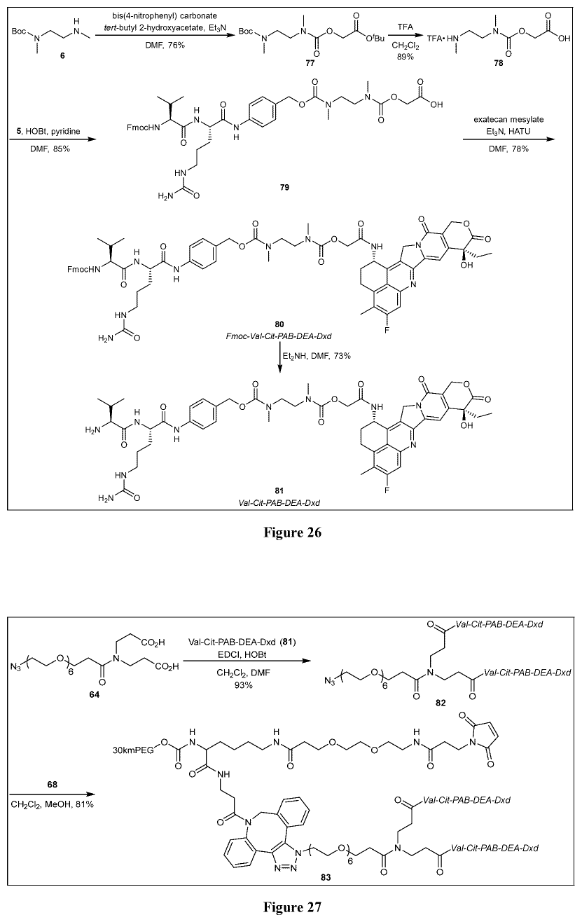

### Example 19. Preparation of Val-Cit-PAB-DEA-Dxd (Compound 81, FIG. 26)

Compound 77: Tert-butyl 2-hydroxyacetate (2.0 g, 15.0 mmol) was added into a mixture of bis(4-nitrophenyl) carbonate (4.6 g, 15.0 mmol) and triethylamine (5.2 mL, 37.5 mmol) in 75 mL DMF at 0° C. The mixture was stirred at room temperature for 2 h before compound 6 (1.7 g, 9.0 mmol) was added into the solution. The mixture was stirred at room temperature for another 2 h. The product was extracted with CH₂Cl₂ (100 mL×3) and the organic layer was washed with water, dried over Na₂SO₄, and concentrated. The crude product was purified by column chromatography to afford compound 77 (2.4 g, 76%) as colorless oil. MS (ESI) m/z [M+Na]⁺ 369.25.

Compound 78: To a solution of compound 77 (2.2 g, 6.3 mmol) in anhydrous CH₂Cl₂ (20 mL), TFA (4.5 mL) was added. The mixture was stirred at room temperature for 2 h. Solvent was removed under vacuum as much as possible at <35° C. The residue was recrystallized from hexane (45 mL). The isolated product was dried under vacuum to yield the product 78 (1.6 g, 89%) as white solid. MS (ESI) m/z [M+H]⁺ 191.30.

Compound 79: Compound 78 (3.3 g, 11.6 mmol) and compound 5 (3.6 g, 4.6 mmol) were dissolved in DMF (50 mL). HOBt (1.2 g, 9.2 mmol) and pyridine (2.5 mL) were then added, and the reaction mixture was stirred at room temperature for 4 h until the reaction was complete. The reaction mixture was cooled to 0° C. and was added to methyl tert-butyl ether (80 mL). The resultant slurry was stirred for 1 h, filtered, washed and dried under vacuum. The crude product was purified by column purification to give compound 79 (2.9 g, 85%) as pale yellow powder. MS (ESI) m/z [M+Na]⁺ 840.43.

Compound 80: To a stirred solution of exatecan mesylate (0.92 g, 1.7 mmol) and triethylamine (0.5 mL, 3.5 mmol) in anhydrous DMF (30 mL) at room temperature under argon, compound 79 (1.4 g, 1.7 mmol) and HATU (0.83 g, 2.2 mmol) were added. The mixture was stirred at room temperature until full conversion was confirmed by HPLC. The mixture was concentrated under vacuum and the residue was purified on silica gel column to give the compound 80 (1.0 g, 78%) as white solid. MS (ESI) m/z [M+H]⁺ 1135.53, [M+Na]⁺ 1157.43.

Compound 81: Diethylamine (6.0 mL) was added to a solution of compound 80 (2.5 g) in DMF (30 mL), then the reaction was allowed to proceed at room temperature for 50 min. The reaction mixture was concentrated under vacuum and the resulting residue was slurried in methyl tert-butyl ether (90 mL) for 2 h. Solids were filtered, washed and dried under vacuum. The crude product was purified on silica gel column to give the compound 81 (1.6 g, 73%) as white solid. MS (ESI) m/z [M+H]⁺ 1013.63, [M+Na]⁺ 1035.58.

### Example 20. Preparation of 20kmPEG-Lys(PEG₂-Mal)-2(Val-Cit-PAB-DEA-Dxd) (Compound 83, FIG. 27)

Compound 82: To a stirred solution of compound 64 (80 mg, 0.15 mmol) in CH₂Cl₂/DMF (5 mL/5 mL) at room temperature under argon, Val-Cit-PAB-DEA-Dxd (81) (334 mg, 0.33 mmol), EDCI (92 mg, 0.48 mmol) and HOBt (20 mg, 0.15 mmol) were added. The mixture was stirred at room temperature until full conversion was confirmed by HPLC. After the reaction was completed, the mixture was concentrated under vacuum. The residue was recrystallized from CH₂Cl₂/methyl tert-butyl ether to give the compound 82 (360 mg, 93%) as white solid. MS (ESI) m/z [M+2H]²⁺ 1257.83, [M+2Na]²⁺ 1279.88.

Compound 83: To a stirred solution of compound 68 (1.8 g, 0.06 mmol) in anhydrous CH₂Cl₂ (8 mL) and MeOH (8 mL), compound 82 (350 mg, 0.14 mmol) was added. The mixture was stirred at room temperature overnight until full conversion was confirmed by HPLC. Solvent was removed and the residue was recrystallized from MeOH/2-propanol (5 mL/40 mL) twice. The residue was dried under vacuum to yield the product 83 (1.5 g, 81%) as white powder.

### Example 21. Preparation of SCAHer2(1)xSCAHer2(2) (Compound 84)

Compound 84: The similar procedures for preparation of compound 52 were used for preparation of compound 84. Amino acid Sequence of SCAHer2(1) x SCAHer2(2) (SEQ ID No. 2) is:

DIQMTQSPSSLSASVGDRVTITCKASQDVSIGVAWYQQKPGKAPK
LLIYSASYRYTGVPSRFSGSGSGTDFTLTISSLQPEDFATYYCQQ
YYIYPYTFGQGTKVEIKRTGGSGGSGGSGGSGGEVQLVESGGGLV
QPGGSLRLSCAASGFTFTDYTMDWVRQAPGKGLEWVADVNPNSGG
SIYNQRFKGRFTLSVDRSKNTLYLQMNSLRAEDTAVYYCARNLGP
SFYFDYWGQGTLVTVSSGCGSGGSGGSGGSGGEVQLVESGGGLVQ
PGGSLRLSCAASGFNIKDTYIHWVRQAPGKGLEWVARIYPTNGYT
RYADSVKGRFTISADTSKNTAYLQMNSLRAEDTAVYYCSRWGGDG
FYAMDYWGQGTLVTVSSGGSGGSGGSGGSGGDIQMTQSPSSLSAS
VGDRVTITCRASQDVNTAVAWYQQKPGKAPKLLIYSASFLYSGVP
SRFSGSRSGTDFTLTISSLQPEDFATYYCQQHYTTPPTFGQGTKV
EIKRTHHHHHH

### Example 22. Preparation of SCAc-Met(1)xSCAc-Met(2) (Compound 85)

Compound 85: The similar procedures for preparation of compound 52 were used for preparation of compound 85. Amino acid Sequence of SCAc-Met(1)xSCAc-Met(2) (SEQ ID No. 6) is:

DIMMSQSPSSLTVSVGEKVTVSCKSSQSLLYTSSQKNYLAWYQQK
PGQSPKLLIYWASTRESGVPDRFTGSGSGTDFTLTITSVKADDLA
VYYCQQYYAYPWTFGGGTKLEIKGGSGGSGGSGGSGGQVQLQQSG
PELVRPGASVKMSCRASGYTFTSYWLHWVKQRPGQGLEWIGMIDP
SNSDTRFNPNFKDKATLNVDRSSNTAYMLLSSLTSADSAVYYCAT
YGSYVSPLDYWGQGTSVTVSSGCGSGGSGGSGGSGGQVQLVESGP
GLVKPSQTLSLTCAVSGGSITTNYYYWSWIRQSPGKGLEWMGVIA
YDGSTDYSPSLKSRTSISRDTSKNQFSLQLSSVTPEDTAVYYCAR
DVRVIATGWATANALDAWGQGTLVTVSSGGSGGSGGSGGSGGQSV
LTQPPSVSGSPGKTVTISCAGTSSDVGYGNYVSWYQQLPGTAPKL
LIFAVSYRASGIPDRFSGSKSGNTAFLTISGLQSEDEADYYCASY
RSSNNAAVFGGGTHLTVLHHHHHH

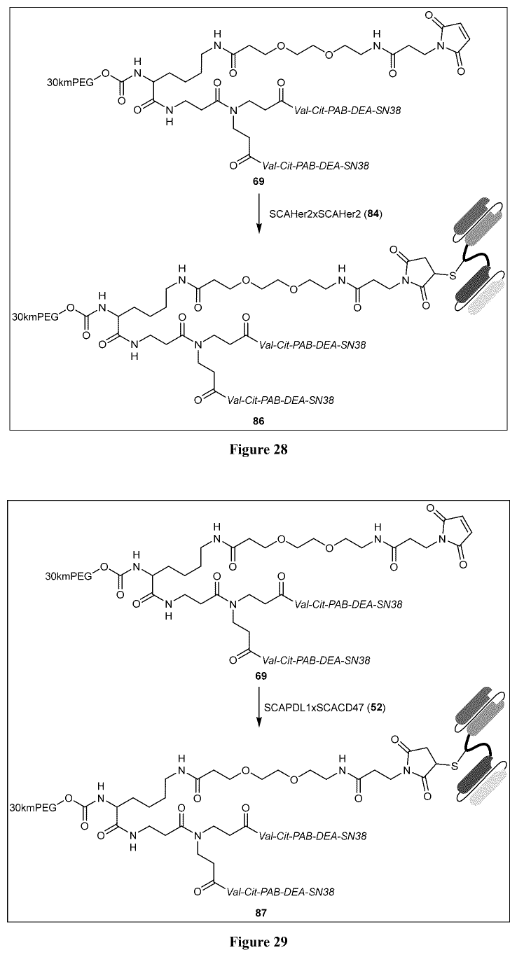

### Example 23. Preparation of 30kmPEG(SCAHer2(1)xSCAHer2(2))-2(Val-Cit-PAB-DEA-SN38) (Compound 86, FIG. 28)

Protein SCAHer2(1)xSCAHer2(2) (84) (20 mg) was treated by reducing agent 2 mM TCEP in PBS buffer (pH=7.4) at room temperature for 30 min before pH adjustment with a pH=4.12 stock solution of 500 mM sodium phosphate buffer. The treated protein was concentrated to 5 mg/mL before pegylation. Pegylation of SCAHer2(1)xSCAHer2(2) was conducted at room temperature for 3 h with 2 to 3 equivalent of compound 69. The reaction was quenched with 10 mM of L-cystine at room temperature for 10 min. Final product compound 86 was purified with hydroxyapatite HA (TOSOH) at the pH 6.8 in 20 mM sodium phosphate buffer. The target compound 86 was confirmed by SEC-HPLC and cell-based activity assay.

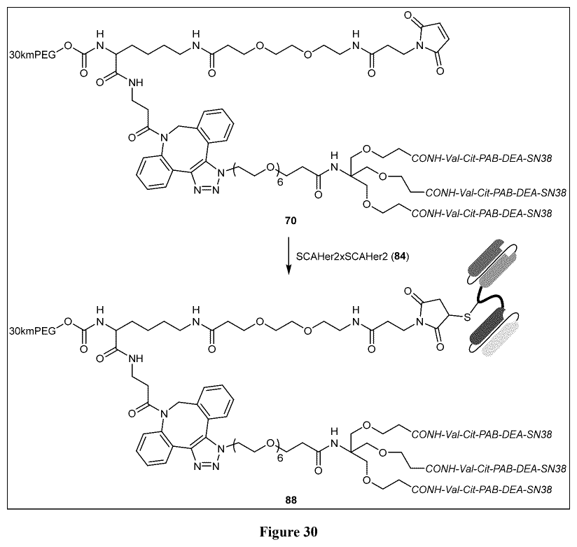

### Example 24. Preparation of 30kmPEG(SCAPDL1xSCACD47)-2(Val-Cit-PAB-DEA-SN38) (Compound 87, FIG. 29)

Compound 87 was made by conjugation of compound 69 [30kmPEG-Lys(PEG2-Mal)-2(Val-Cit-PAB-DEA-SN38)] with protein 52 with similar procedures as for preparation of compound 86.

### Example 25. Preparation of 30kmPEG(SCAHer2(1)xSCAHer2(2))-3(Val-Cit-PAB-DEA-SN38) (Compound 88, FIG. 30)

Compound 88 was made by conjugation of compound 70 [30kmPEG-Lys(PEG2-Mal)-3(Val-Cit-PAB-DEA-SN38)] with protein 84 with similar procedures as for preparation of compound 86.

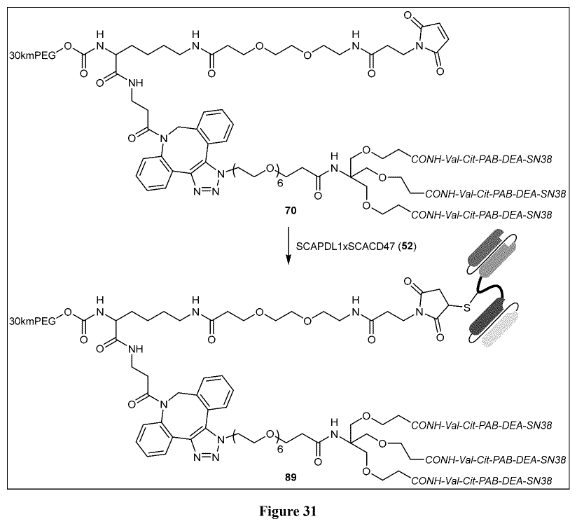

### Example 26. Preparation of 30kmPEG(SCAPDL1xSCACD47)-3(Val-Cit-PAB-DEA-SN38) (Compound 89, FIG. 31)

Compound 89 was made by conjugation of compound 70 [30kmPEG-Lys(PEG2-Mal)-3(Val-Cit-PAB-DEA-SN38)] with protein 52 with similar procedures as for preparation of compound 86.

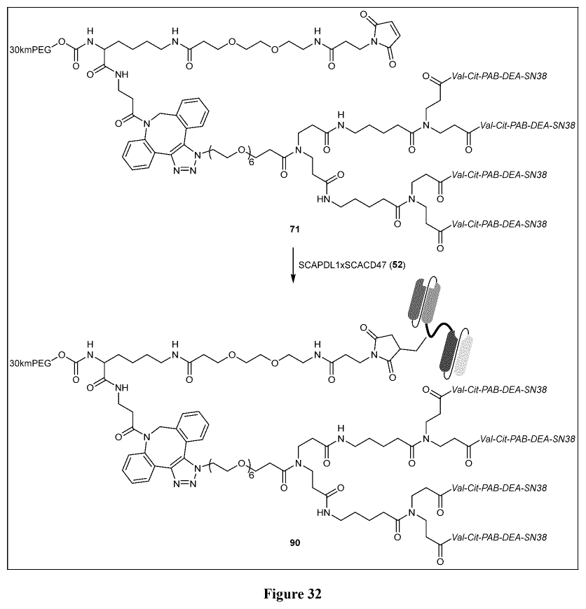

### Example 27. Preparation of 30kmPEG(SCAPDL1xSCACD47)-4(Val-Cit-PAB-DEA-SN38) (Compound 90, FIG. 32)

Compound 90 was made by conjugation of compound 71 [30kmPEG-Lys(PEG₂-Mal)-4(Val-Cit-PAB-DEA-SN38)] with protein 52 with similar procedures as for preparation of compound 86.

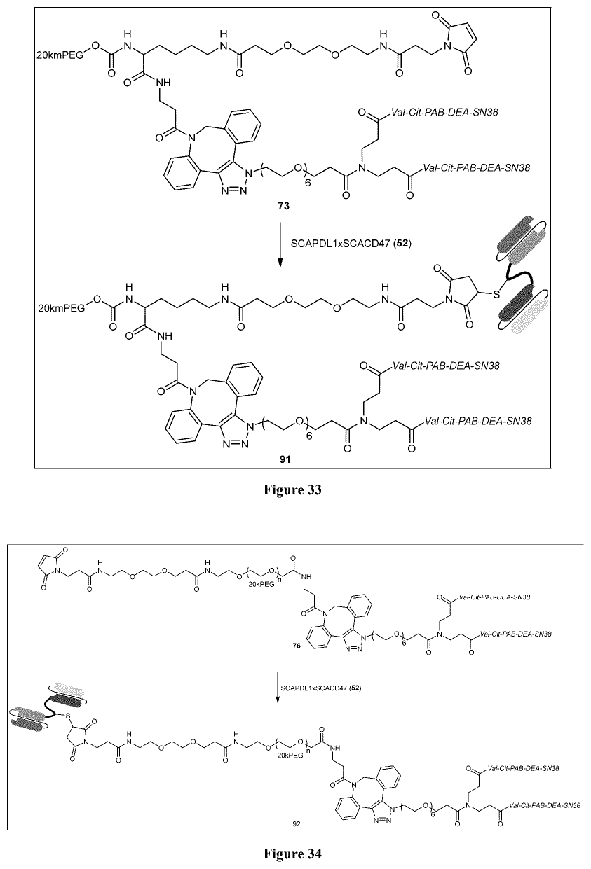

### Example 28. Preparation of 20kmPEG(SCAPDL1xSCACD47)-2(Val-Cit-PAB-DEA-SN38) (Compound 91, FIG. 33)

Compound 91 was made by conjugation of compound 73 [20kmPEG-Lys(PEG₂-Mal)-2(Val-Cit-PAB-DEA-SN38)] with protein 52 with similar procedures as for preparation of compound 86.

### Example 29. Preparation of SCAPDL1xSCACD47-20kPEG-2(Val-Cit-PAB-DEA-SN38) (Compound 92, FIG. 34)

Compound 92 was made by conjugation of compound 76 [(PEG₂-Mal)-20kPEG-2(Val-Cit-PAB-DEA-SN38)] with protein 52 with similar procedures as for preparation of compound 86.

### Example 30. Preparation of 30kmPEG(SCAPDL1xSCACD47)-2(Val-Cit-PAB-DEA-Dxd) (Compound 93, FIG. 35)

Compound 93 was made by conjugation of compound 83 [30kmPEG-Lys(PEG₂-Mal)-2(Val-Cit-PAB-DEA-Dxd)]with protein 52 with similar procedures as for preparation of compound 86.

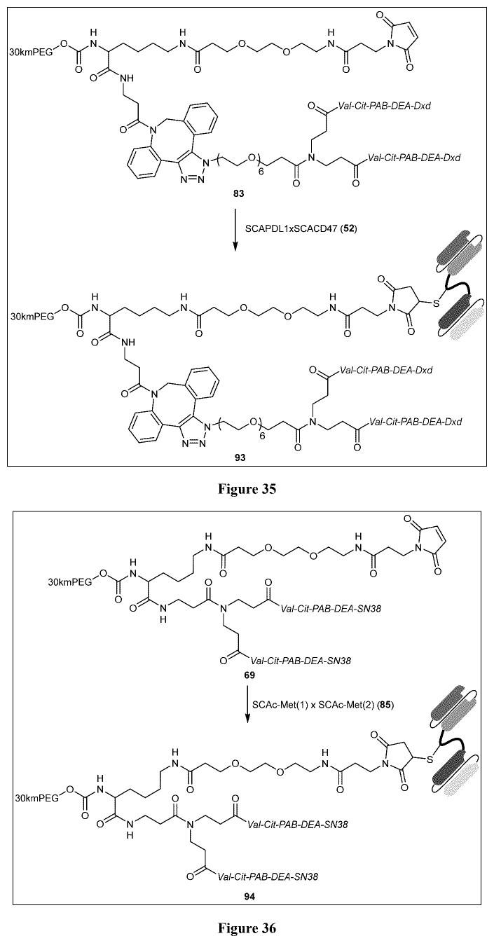

### Example 31. Preparation of 30kmPEG(SCAc-Met(1)xSCAc-Met(1))-2(Val-Cit-PAB-DEA-SN38) (Compound 94, FIG. 36)

Compound 94 was made by conjugation of compound 69 [30kmPEG-Lys(PEG₂-Mal)-2(Val-Cit-PAB-DEA-SN38)] with protein 85 with similar procedures as for preparation of compound 86.

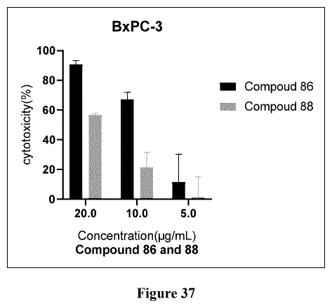

### Example 32. In Vitro Cytotoxicity of Compound 86 and Compound 88 to Tumor Cell Line (FIG. 37)

To verify cytotoxic activity, HER2 expression positive tumor cell line BxPC-3 was selected for viability analysis in vitro. Cells were seeded in a 96-well plate at 3×10⁵ cells/well and treated with indicated doses of compound 86 and compound 88. Cell viability was determined by Cell Counting Kit-8 (CCK-8) according to the manufacturer's instructions. The inhibition of cell proliferation calculated with the following equation: cytotoxicity %=(1-OD sample/OD control)×100%. Data were analyzed with GraphPad Prism software and are presented as percent growth inhibition relative to the untreated control. The results were shown in FIG. 37.

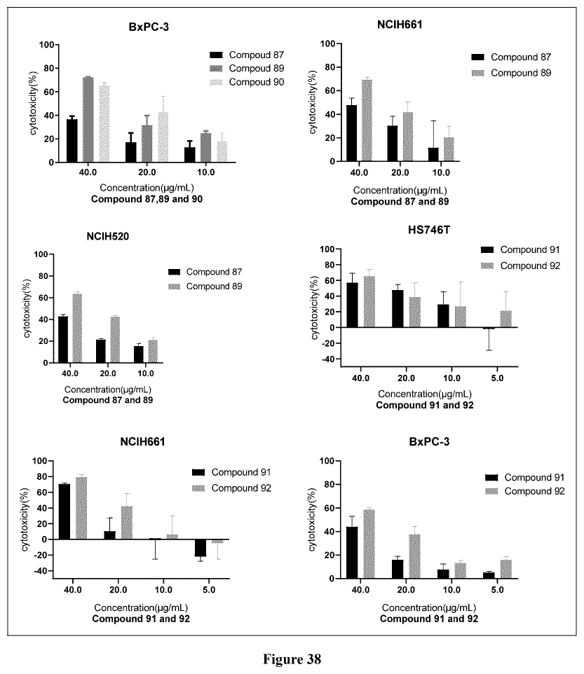

### Example 33. In Vitro Cytotoxicity of Compounds 87, 89, 90, 91 and 92 to Tumor Cell Lines (FIG. 38)

To verify cytotoxic activity, a set of CD47/PD-L1 expression positive tumor cell lines BxPC-3, NCIH661, NCIH520, and HS746T were selected for viability analysis in vitro. Cells were seeded in a 96-well plate at 3×10⁵ cells/well and treated with indicated doses of compounds 87, 89, 90, 91 and 92. Cell viability was determined by Cell Counting Kit-8 (CCK-8) according to the manufacturer's instructions. The inhibition of cell proliferation calculated with the following equation: cytotoxicity %=(1-OD sample/OD control)×100%. Data were analyzed with GraphPad Prism software and are presented as percent growth inhibition relative to the untreated control. The results were shown in FIG. 38.

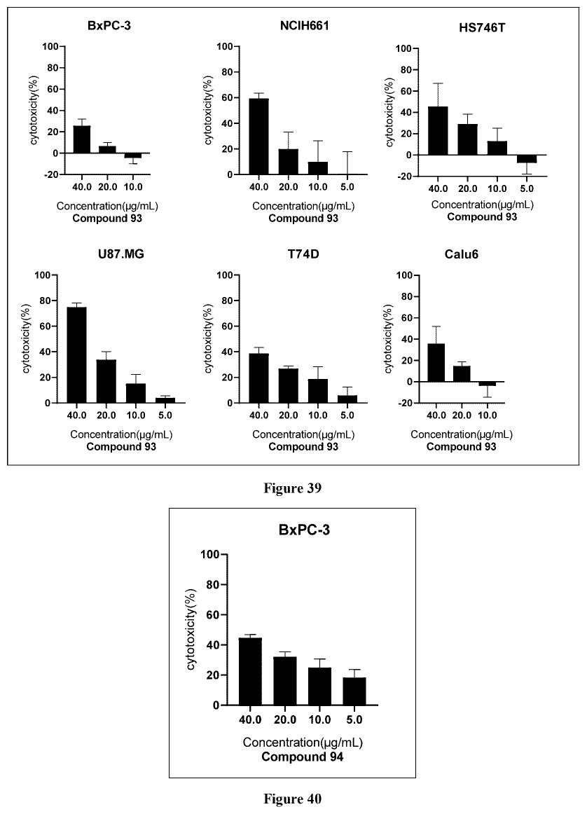

### Example 34. In Vitro Cytotoxicity of Compound 93 to Tumor Cell Line (FIG. 39)

To verify cytotoxic activity, a set of CD47/PD-L1 expression positive tumor cell line BxPC-3, NCIH661, HS746T, U87.MG, T74D and Calu6 were selected for viability analysis in vitro. Cells were seeded in a 96-well plate at 3×10⁵ cells/well and treated with indicated doses of compound 93. Cell viability was determined by Cell Counting Kit-8 (CCK-8) according to the manufacturer's instructions. The inhibition of cell proliferation calculated with the following equation: cytotoxicity %=(1−OD sample/OD control)×100%. Data were analyzed with GraphPad Prism software and are presented as percent growth inhibition relative to the untreated control. The results were shown in FIG. 39.

### Example 35. In Vitro Cytotoxicity of Compound 94 to Tumor Cell Lines (FIG. 40)

To verify cytotoxic activity, c-MET expression positive tumor cell line BxPC-3 was selected for viability analysis in vitro. Cells were seeded in a 96-well plate at 3×10⁵ cells/well and treated with indicated doses of compound 94. Cell viability was determined by Cell Counting Kit-8 (CCK-8) according to the manufacturer's instructions. The inhibition of cell proliferation calculated with the following equation: cytotoxicity %=(1−OD sample/OD control)×100%. Data were analyzed with GraphPad Prism software and are presented as percent growth inhibition relative to the untreated control. The results were shown in FIG. 40.

1. A compound of the Formula (Ib) 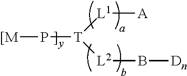
wherein
P is a non-immunogenic polymer;
M is H or a terminal capping group selected from C₁₋₅₀ alkyl and aryl, wherein one or more carbons of said alkyl are optionally replaced with a heteroatom;
y is 1;
A is an antibody fragment or an antigen binding fragment thereof;
T is a multifunctional small molecule linker moiety;
each of Lⁱ and L² is independently a hetero- or homobifunctional linker;
each of a and b is an integer selected from 0-10;
B is a branched linker, wherein each branch has an amino acid sequence or carbohydrate moiety or a disulfide bond linked to one or more self-immolating spacer, wherein cleavage of the amino acid sequence or carbohydrate moiety or a disulfide bond by an enzyme triggers self-immolating mechanism to release hydroxyl bearing drug D, or each branch has a cleavable bond, wherein the cleavage of the cleavable bond releases hydroxyl bearing drug D;
each of D is independently a cytotoxic hydroxyl-bearing small molecule or peptide, wherein the hydroxyl group of D is linked to B; and
n is an integer selected from 1-25.

2. The compound of claim 1, wherein T is a tri-functional linker derived from a molecule with three functional groups independently selected from hydroxyl, amino, hydrazinyl, azide, alkene, alkyne, aldehyde, ketone, ester, carboxylic acid, anhydride, acyl halide, thiol, disulfide, nitrile, epoxide, imine, nitro and halide, and wherein the linkage between T and (L¹)ₐ and the linkage between T and (L²)_{b} are the same or different, alternatively, wherein T is 1,3-diamino-2-propanol, triethanolamine, lysine, aspartic acid, glutamic acid, serine or tyrosine.

3. (canceled)

4. The compound of claim 1, wherein one of the functional group at the linker terminal of (L¹)ₐ is capable of site-specific conjugation with A, and is selected from the group consisting of thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and Iodine.

5. The compound of claim 1, wherein the antibody fragment is a mono-specific or multi-specific antibody fragment, a mono-specific or multi-specific single chain antibody, a mono-specific or multi-specific nanobody (a single domain antibody), or a mono-specific or multi-specific antigen binding domain thereof;
alternatively, wherein the antibody is a mono-specific single chain antibody;
alternatively, wherein the mono-specific single chain antibody binds to a tumor associated antigen (TAA) such as Her2, cMet, PDL1 or CD47;
alternatively, wherein the mono-specific single chain antibody has two binding domains binding to Her2;
alternatively, wherein the mono-specific single chain antibody has an amino acid sequence as shown in SEQ ID No. 3;
alternatively, wherein the antibody is a bispecific antibody, e.g. a bispecific single chain antibody;
alternatively, wherein the two binding domains of the bispecific antibody bind to the same tumor associated antigen (TAA), bind to two different TAAs, or bind to a TAA and an antigen expressed on T cells (e.g. a component of T cell receptor) or NK cells;
alternatively, wherein the antibody is an anti-PDL1 x anti-CD47 single chain bispecific antibody;
alternatively, wherein the antibody has an amino acid sequence as shown in SEQ ID No. 1;
alternatively, wherein the antibody is an anti-HER2(1) x anti-HER(2) single chain bispecific antibody;
alternatively, wherein the antibody has an amino acid sequence as shown in SEQ ID No. 2;
alternatively, wherein the antibody is an anti-cMet(1) x anti-cMet(2) single chain bispecific antibody;
alternatively, wherein the antibody has an amino acid sequence as shown in SEQ ID No. 6;
alternatively, wherein the two binding domains of the mono-specific single chain antibody are linked via a peptide linker, and wherein the linker comprises a cysteine or an unnatural amino acid residue for site-specific conjugation of the antibody to (L¹)^{a};
alternatively, wherein the two binding domains of the bispecific single chain antibody are linked via a peptide linker, and wherein the linker comprises a cysteine or an unnatural amino acid residue for site-specific conjugation of the antibody to (L¹)ₐ;
alternatively, wherein the unnatural amino acid residue is selected from the group consisting of genetically-encoded alkene lysines (such as N6-(hex-5-enoyl)-L-lysine), 2-amino-8-oxononanoic acid, m- or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino)hexanoic acid, azidohomoalanine, pyrrolysine analogue N6-((prop-2-yn-1-yloxy)carbonyl)-L-lysine, (S)-2-amino-6-pent-4-ynamidohexanoic acid, (S)-2-amino-6-((prop-2-ynyloxy)carbonylamino)hexanoic acid, (S)-2-amino-6-((2-azidoethoxy)carbonylamino)hexanoic acid, p-azidophenylalanine, para-azidophenylalanine, Nε-Acryloyl-l-lysine, Nε-5-norbornene-2-yloxycarbonyl-l-lysine, N-ε-(Cyclooct-2-yn-1-yloxy)carbonyl)-L-lysine, N-ε-(2-(Cyclooct-2-yn-1-yloxy)ethyl) carbonyl-L-lysine, and genetically encoded tetrazine amino ccid (such as 4-(6-methyl-s-tetrazin-3-yl)aminophenylalanine).

6-20. (canceled)

21. The compound of claim 1, wherein the hydroxyl-bearing drug D is selected from a DNA crosslinker agent, a microtubule inhibitor, a DNA alkylator, a topoisomerase inhibitor, protein degrader, STING agonist or a combination thereof;
alternatively, wherein the hydroxyl-bearing drug D is selected from vinca alkaloid, laulimalide, colchicine, tubulysins, cryptophycins, hemiasterlin, cemadotin, rhizoxin, discodermolide, taccalonolide A or B or AF or AJ, taccalonolide AI-epoxide, CA-4, epothilone A and B, taxane, paclitaxel, docetaxel, epothilone, iSGD-1882, centanamycin, PNU-159682, uncialamycin, indolinobenzodiazepine dimers, β-amanitin, amatoxins, thailanstatins, calicheamicin, anthracycline, daunomycin, larotaxel, tesetaxel, ortataxel, CC-1065, Dxd, SN38, topotecan, CPT-11, camptothecin, exatecan, rubitecan, bryostatin, callystatin, bizelesin, duocarmycin, eleutherobin, pancratistatin, sarcodictyin, spongistatin, estramustine, prednimustine, chlorozotocin, ranimustine, calicheamicin, dynemicin, esperamicin, neocarzinostatin chromophore, aclacinomysins, azithromycin, bleomycins, caminomycin, carzinophilin, chromomycins, daunorubicin, detorubicin, doxorubicin, epirubicin, esorubicin, idarubicin, marcellomycin, mycophenolic acid, nogalamycin, peplomycin, puromycin, quelamycin, rodorubicin, streptonigrin, streptozocin, tubercidin, ubenimex, zinostatin, fludarabine, ancitabine, azacytidine, 6-azauridine, carmofur, cytarabine (cytosine arabinoside, ara-C), gemcitabine, capecitabine, dideoxyuridine, doxifluridine, enocitabine, floxuridine, calusterone, epitiostanol, trilostane, elliptinium acetate, maytansinoids, ansamitocins, mitoxantrone, mopidamol, pentostatin, pirarubicin, etoposide, podophyllotoxin, rhizoxin, tenuazonic acid, T-2 mycotoxin, verracurin A, roridin A, anguidine, vindesine, mannomustine, mitobronitol, mitolactol, vinblastine, mitoxantrone, vincristine, vinorelbine, teniposide, xeloda, raloxifene, 4-hydroxytamoxifen, estradiol, trioxifene, keoxifene, LY117018, onapristone, bicalutamide, leuprolide, goserelin or its pharmaceutically acceptable salts, acids or derivatives thereof, or a combination thereof;
alternatively, wherein D is selected from duocarmycin, Dxd, SN38, topotecan, CPT-11, camptothecin, exatecan, rubitecan or a derivate thereof, or a combination thereof.

22-23. (canceled)

24. The compound of claim 1, wherein the non-immunogenic polymer is polyethylene glycol (PEG);
alternatively, wherein the PEG is a liner PEG or a branched PEG;
alternatively, wherein at least one terminal of the PEG is capped with methyl or a low molecule weight alkyl;
alternatively, wherein a total molecule weight of the PEG is from 3000 to 100000;
alternatively, wherein the PEG is linked to the trifunctional or tetrafunctional or any other cyclic or noncyclic multifunctional moiety T (e.g. a lysine) through a permanent bond or a cleavable bond.

25-28. (canceled)

29. A compound of the Formula (Ic) 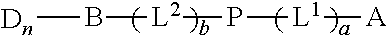
wherein
P is a liner PEG;
A is an antibody fragment or an antigen binding fragment thereof;
each of L¹ and L² is independently a bifunctional linker;
each of a and b is an integer selected from 0-10;
B is a branched linker, wherein each branch has an amino acid sequence or carbohydrate moiety or a disulfide bond linked to one or more self-immolating spacer, wherein cleavage of the amino acid sequence or carbohydrate moiety or a disulfide bond by an enzyme triggers self-immolating mechanism to release hydroxyl-bearing drug D, or each branch has a cleavable bond, wherein cleavage of the cleavable bond releases hydroxyl-bearing drug D or its derivative;
each of D is independently a cytotoxic hydroxyl-bearing small molecule or peptide, wherein the hydroxyl group of D is linked to B;
n is an integer selected from 1-25.

30. The compound of claim 29, wherein the functional group at the linker terminal of (L¹)ₐ is capable of site-specific conjugation with A, and is selected from the group consisting of thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and iodine.

31. The compound of claim 29, wherein the antibody fragment is a mono-specific or multi-specific antibody fragment, a mono-specific or multi-specific single chain antibody, a mono-specific or multi-specific nanobody (a single domain antibody), or a mono-specific or multi-specific antigen binding domain thereof;
alternatively, wherein the antibody is a mono-specific single chain antibody, optionally wherein the mono-specific single chain antibody binds to a tumor associated antigen (TAA) such as Her2, cMet, PDL1 or CD47;
alternatively, wherein the mono-specific single chain antibody has two binding domains binding to Her2;
alternatively, wherein the mono-specific single chain antibody has an amino acid sequence as shown in SEQ ID No. 3;
alternatively, wherein the antibody is a bispecific antibody, e.g. a bispecific single chain antibody;
alternatively, wherein the two binding domains of the bispecific antibody bind to the same tumor associated antigen (TAA), bind to two different TAAs, or bind to a TAA and an antigen expressed on T cells (e.g. a component of T cell receptor) or NK cells;
alternatively, wherein the antibody is an anti-PDL1 x anti-CD47 single chain bispecific antibody;
alternatively, wherein the antibody has an amino acid sequence as shown in SEQ ID No. 1;
alternatively, wherein the antibody is an anti-HER2(1) x anti-HER2(2) single chain bispecific antibody;
alternatively, wherein the antibody has an amino acid sequence as shown in SEQ ID No. 2;
alternatively, wherein the antibody is an anti-cMet(1) x anti-cMET(2) single chain bispecific antibody;
alternatively, wherein the antibody has an amino acid sequence as shown in SEQ ID No. 6;
alternatively, wherein the two binding domains of the mono-specific single chain antibody are linked via a peptide linker, and wherein the linker comprises a cysteine or an unnatural amino acid residue for site-specific conjugation of the antibody to (L′)ₐ;
alternatively, wherein the two binding domains of the bispecific single chain antibody are linked via a peptide linker, and wherein the linker comprises a cysteine or an unnatural amino acid residue for site-specific conjugation of the antibody to (L′)ₐ;
alternatively, wherein the unnatural amino acid residue is selected from the group consisting of genetically-encoded alkene lysines (such as N6-(hex-5-enoyl)-L-lysine), 2-Amino-8-oxononanoic acid, m- or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino)hexanoic acid, azidohomoalanine, pyrrolysine analogue N6-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, p-azidophenylalanine, para-azidophenylalanine, Nε-Acryloyl-l-lysine, Nε-5-norbornene-2-yloxycarbonyl-l-lysine, N-ε-(Cyclooct-2-yn-1-yloxy)carbonyl)-L-lysine, N-ε-(2-(Cyclooct-2-yn-1-yloxy)ethyl) carbonyl-L-lysine, and genetically nncoded tetrazine amino acid (such as 4-(6-methyl-s-tetrazin-3-yl)aminophenylalanine).

32-45. (canceled)

46. The compound of claim 29, wherein the hydroxyl-bearing drug D is selected from a DNA crosslinker agent, a Microtubule inhibitor, a DNA alkylator, a Topoisomerase inhibitor, protein degrader, STING agonist or a combination thereof;
alternatively, wherein D is selected from vinca alkaloid, laulimalide, colchicine, tubulysins, cryptophycins, hemiasterlin, cemadotin, rhizoxin, discodermolide, taccalonolide A or B or AF or AJ, taccalonolide AI-epoxide, CA-4, epothilone A and B, taxane, paclitaxel, docetaxel, epothilone, iSGD-1882, centanamycin, PNU-159682, uncialamycin, indolinobenzodiazepine dimers, (3-amanitin, amatoxins, thailanstatins, calicheamicin, anthracycline, daunomycin, larotaxel, tesetaxel, ortataxel, CC-1065, Dxd, SN38, topotecan, CPT-11, camptothecin, exatecan, rubitecan, bryostatin, callystatin, bizelesin, duocarmycin, eleutherobin, pancratistatin, sarcodictyin, spongistatin, estramustine, prednimustine, chlorozotocin, ranimustine, calicheamicin, dynemicin, esperamicin, neocarzinostatin chromophore, aclacinomysins, azithromycin, bleomycins, caminomycin, carzinophilin, chromomycins, daunorubicin, detorubicin, doxorubicin, epirubicin, esorubicin, idarubicin, marcellomycin, mycophenolic acid, nogalamycin, peplomycin, puromycin, quelamycin, rodorubicin, streptonigrin, streptozocin, tubercidin, ubenimex, zinostatin, fludarabine, ancitabine, azacytidine, 6-azauridine, carmofur, cytarabine (cytosine arabinoside, ara-C), gemcitabine, capecitabine, dideoxyuridine, doxifluridine, enocitabine, floxuridine, calusterone, epitiostanol, trilostane, elliptinium acetate, maytansinoids, ansamitocins, mitoxantrone, mopidamol, pentostatin, pirarubicin, etoposide, podophyllotoxin, rhizoxin, tenuazonic acid, T-2 mycotoxin, verracurin A, roridin A, anguidine, vindesine, mannomustine, mitobronitol, mitolactol, vinblastine, mitoxantrone, vincristine, vinorelbine, teniposide, xeloda, raloxifene, 4-hydroxytamoxifen, Estradiol, trioxifene, keoxifene, LY117018, onapristone, bicalutamide, leuprolide, goserelin or its pharmaceutically acceptable salts, acids or derivatives thereof, or a combination thereof;
alternatively, wherein D is selected from duocarmycin, Dxd, SN38, topotecan, CPT-11, camptothecin, exatecan, rubitecan or a derivate thereof, or a combination thereof.

47-48. (canceled)

49. The compound of claim 29, wherein a total molecule weight of the PEG is from 3000 to 100000 Dalton.

50. The compound of claim 29, wherein each of L¹ and L² is independently selected from the group consisting of:
—(CH₂)ₐXY(CH₂)_{b}—,
—X(CH₂)ₐO(CH₂CH₂O)_{c}(CH₂)_{b}Y—,
—(CH₂)ₐheterocyclyl-,
—(CH₂)ₐX—,
—X(CH₂)ₐY—,
—W₁—(CH₂)ₐC(O)NR₁(CH₂)_{b}O(CH₂CH₂O)_{c}(CH₂)_{d}C(O)—,
—C(O)(CH₂)ₐO(CH₂CH₂O)_{b}(CH₂)_{c}W₂C(O)(CH₂)_{d}NR₁—, and
—W₃—(CH₂)ₐC(O)NR₁(CH₂)_{b}O(CH₂CH₂O)_{c}(CH₂)_{d}W₂C(O)(CH₂)ₑC(O)—,
wherein each of a, b, c, d and e is independently an integer selected from 0 to 25; each of X and Y is independently selected from C(═O), NR2, S, O, N3, CR₃R₄, a DBCO-based moiety or Null; each of R₁, R₂, R₃ and R₄independently represents hydrogen, C₁₋₁₀ alkyl or (CH₂)₁₋₁₀C(═O); W₁ and/or W₃ is derived from a maleimido-based moiety and W₂ represents a triazolyl or a tetrazolyl containing group; and the heterocyclyl group is selected from a maleimido-derived moiety or a tetrazolyl-based or a triazolyl-based moiety;
alternatively, wherein each of (L¹)ₐ and (L²)_{b} is independently selected from: 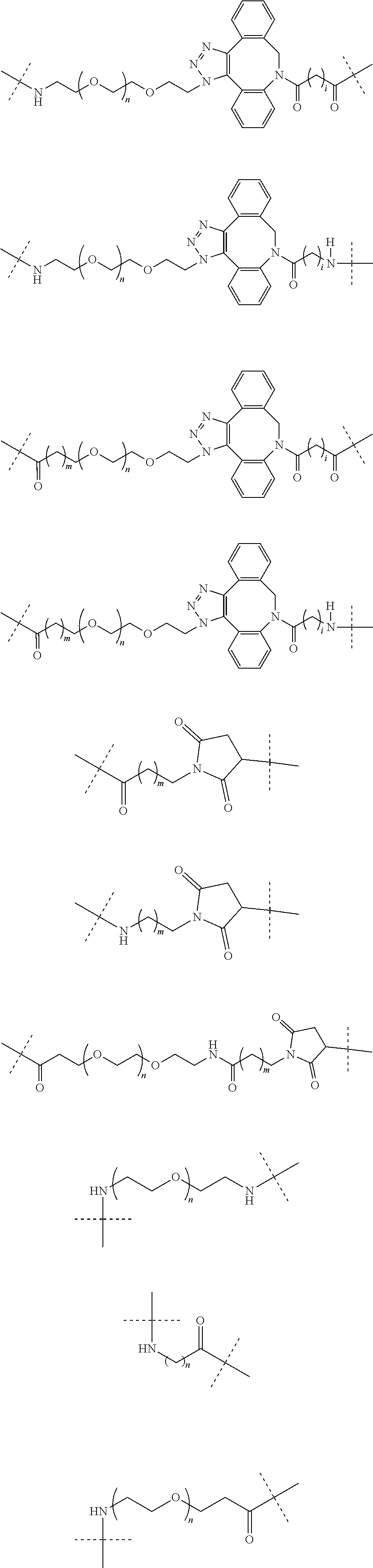
wherein each of i, m and n is independently an integer selected from 0 to 20;
alternatively, wherein the branched linker B comprise an extension spacer (optional), a trigger unit, one or more self-immolating spacer or any combination thereof, wherein the trigger unit is an amino acid sequence or a β-glucoronide or β-galactoside trigger moiety cleavable by an enzyme such as cathepsin B, plasmin, matrix metalloproteinases (MMPs), β-glucuronidases, β-galactosidases: a pH liable linker that can release the hydroxyl-bearing drug D or its derivatives at acidic pH conditions, or a disulfide bond linker that can trigger the release of the hydroxyl-bearing drug D or its derivatives by glutathione, thioredoxin family members (WCGH/PCK) or thio reductase;
alternatively, wherein the branched linker B is selected from 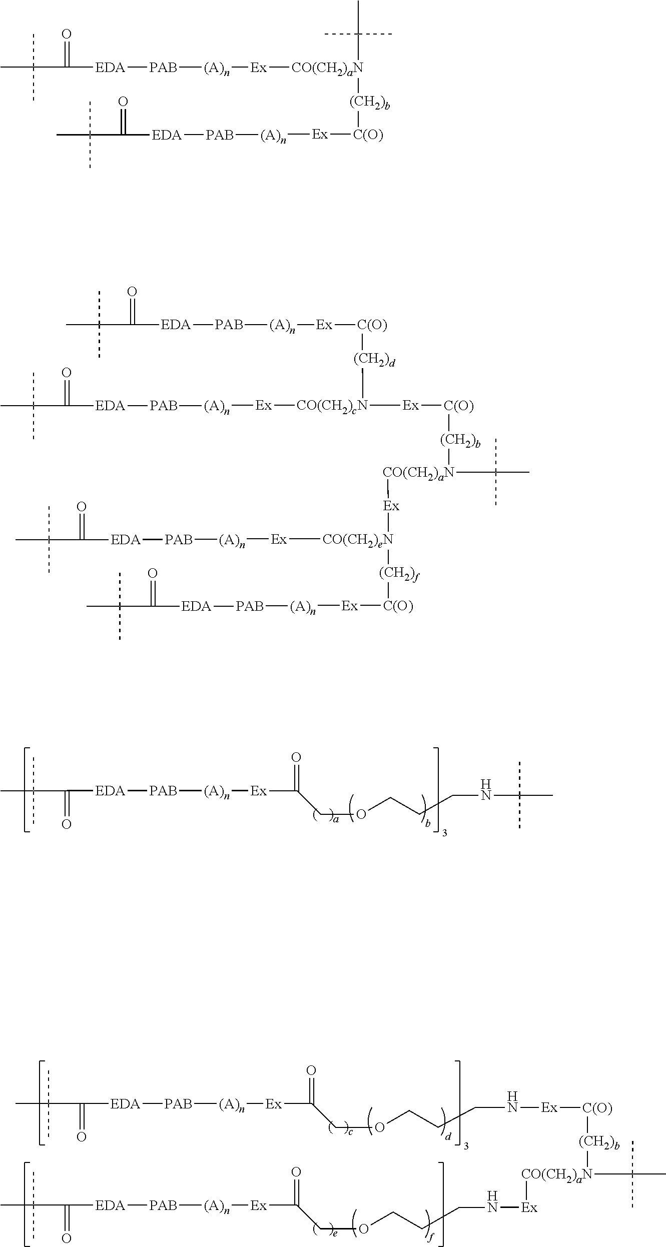
wherein:
each of a, b, c, d, e and f is independently an integer selected from 1-25;
(A)ₙ is a trigger unit of amino acid sequence such as Val-Cit, Val-Ala, Val-Lys, Phe-Lys, Phe-Cit, Phe-Arg, Phe-Ala, Ala-Lys, Leu-Cit, Ile-Cit, Trp-Cit, D-Phe-Phe-Lys, Phe-Phe-Lys, Gly-Phe-Lys, Gly-Phe-Leu-Gly, Gly-Gly-Phe-Gly or Ala-Leu-Ala-Leu;
PAB is para-aminobenzyl alcohol;
EDA is —NR¹(CH₂)ₘNR²—, wherein m is 2 or 3, each of R¹ and R² is independently selected from H, a low molecule weight alkyl or —(CH₂CH₂O)ₗ—CH₃, wherein 1 is an integer selected from 1-10;
each of Ex is an extension spacer comprising a linker chain that is independently selected from:
—NR¹(CH₂)ₓO(CH₂CH₂O)_{y}(CH₂)_{z}C(O)—,
—C(O)(CH₂)ₓNR¹—,
—NR¹(CH₂)ₓO(CH₂CH₂O)_{y}(CH₂)_{z}NR²—,
—NR¹(CH₂)ₓNR²—,
—NR¹(CH₂)ₓO(CH₂CH₂O)_{y}(CH₂)_{z}O—,
—O(CH₂)ₓNR¹—,
—C(O)(CH₂)ₓO—,
—O(CH₂)ₓO(CH₂CH₂O)_{y}(CH₂)_{z}C(O)—,
—C(O)(CH₂)ₓO(CH₂CH₂O)_{y}(CH₂)_{z}C(O)—,
—C(O)(CH₂)ₓC(O)—,
or Null,
wherein each of x, y, and z is independently an integer selected from 0 to 25; and each of R¹ and R²independently represents hydrogen or a C₁₋₁₀alkyl group:
alternatively, wherein the branched linker B is selected from

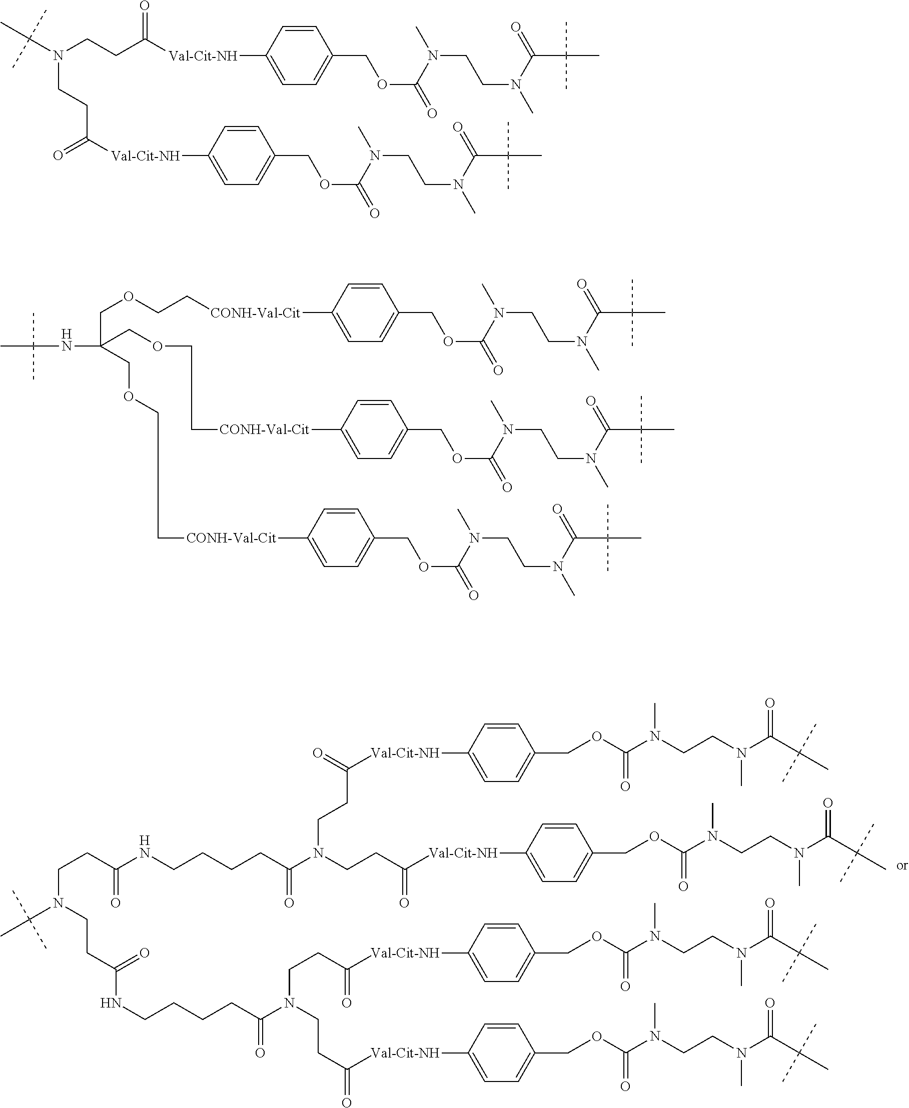

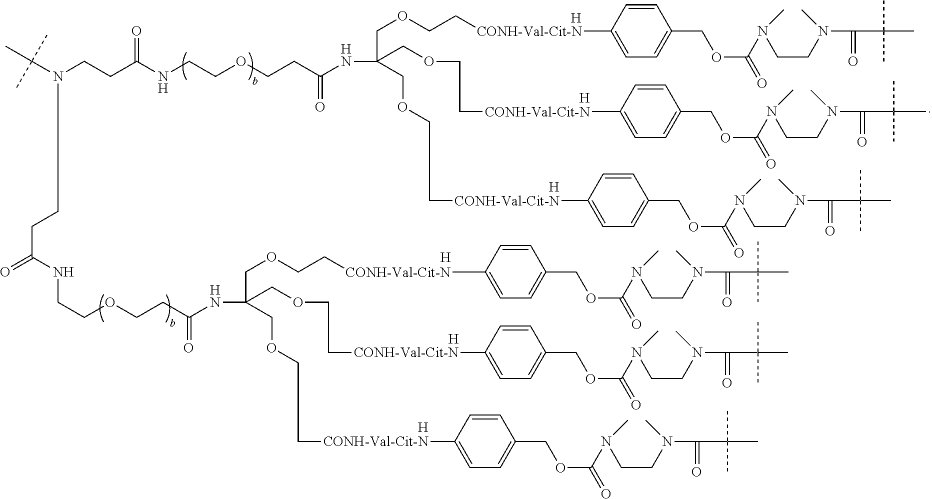

51-57. (canceled)

58. (canceled)

59. A method of preparing a compound of claim 1, comprising:
a step of preparation of the non-immunogenic modified (e.g. PEGylated) hydroxyl-bearing drug conjugate with a free functional group for site-specific conjugation;
b) a step of site-specific conjugation of the non-immunogenic modified (e.g. PEGylated) hydroxyl-bearing drug conjugate to an antibody to provide a compound of the Formula (Ib) or (Ic).

60. A pharmaceutical formulation comprising an effective amount of the compound of claim 1 and a pharmaceutically acceptable salt, carrier or excipient.

61. A method for treating a cancer selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer, head and neck cancer and endometrial cancer, comprising administering to a subject an effective amount of the compound of claim 1.

62. A method for treating a cancer selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer and endometrial cancer, comprising administering to a subject an effective amount of the compound of claim 1 in combination with an effective amount of another anticancer agent or immunosuppressant agent.

63. The compound of claim 1, wherein each of L¹ and L² is independently selected from the group consisting of:
—(CH₂)ₐXY(CH₂)_{b}—,
—X(CH₂)ₐO(CH₂CH₂O)_{c}(CH₂)_{b}Y—,
—(CH₂)ₐheterocyclyl-,
—(CH₂)ₐX—,
—X(CH₂)ₐY—,
—W₁—(CH₂)ₐC(O)NR₁(CH₂)_{b}O(CH₂CH₂O)_{c}(CH₂)_{d}C(O)—,
—C(O)(CH₂)ₐO(CH₂CH₂O)_{b}(CH₂)_{c}W₂C(O)(CH₂)_{d}NR¹—, and
—W₃—(CH₂)ₐC(O)NR₁(CH₂)_{b}O(CH₂CH₂O)_{c}(CH₂)_{d}W₂C(O)(CH₂)ₑC(O)—,
wherein each of a, b, c, d and e is independently an integer selected from 0 to 25; each of X and Y is independently selected from C(═O), NR₂, S, O, N3, CR₃R₄, a DBCO-based moiety or Null; each of R₁, R₂, R₃ and R₄independently represents hydrogen, C₁₋₁₀ alkyl or (CH₂)₁₋₁₀C(═O); W₁ and/or W₃ is derived from a maleimido-based moiety and W₂ represents a triazolyl or a tetrazolyl containing group; and the heterocyclyl group is selected from a maleimido-derived moiety or a tetrazolyl-based or a triazolyl-based moiety;
alternatively, wherein each of (L¹)ₐ and (L²)_{b} is independently selected from: 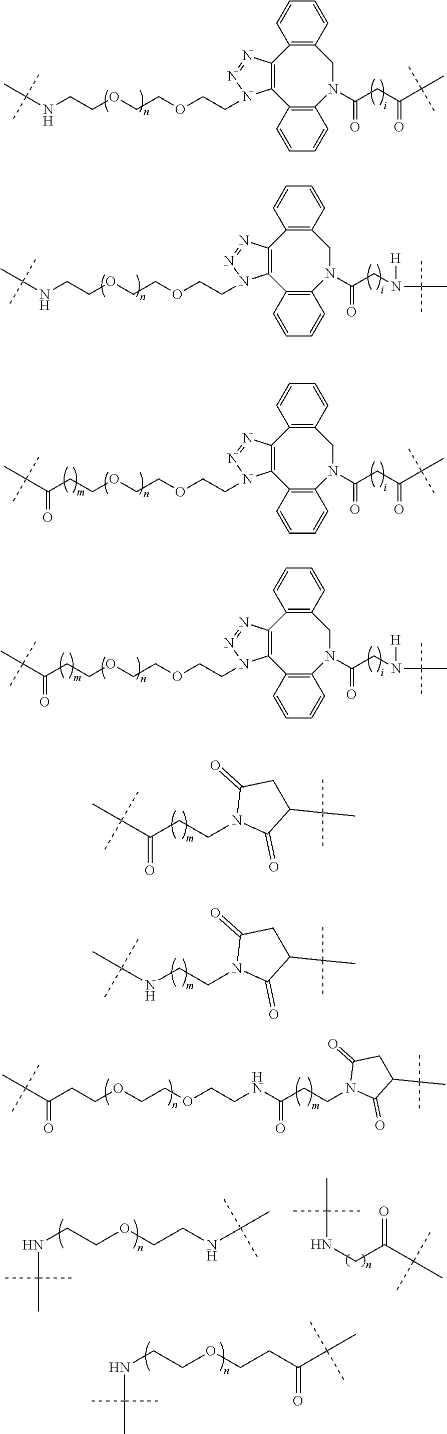
wherein each of i, m and n is independently an integer selected from 0 to 20;
alternatively, wherein the branched linker B comprise an extension spacer (optional), a trigger unit, one or more self-immolating spacer or any combination thereof, wherein the trigger unit is an amino acid sequence or a β-glucoronide or β-galactoside trigger moiety cleavable by an enzyme such as cathepsin B, plasmin, matrix metalloproteinases (MMPs), β-glucuronidases, β-galactosidases; a pH liable linker that can release the hydroxyl-bearing drug D or its derivatives at acidic pH conditions, or a disulfide bond linker that can trigger the release of the hydroxyl-bearing drug D or its derivatives by glutathione, thioredoxin family members (WCGH/PCK) or thio reductase;
alternatively, wherein the branched linker B is selected from 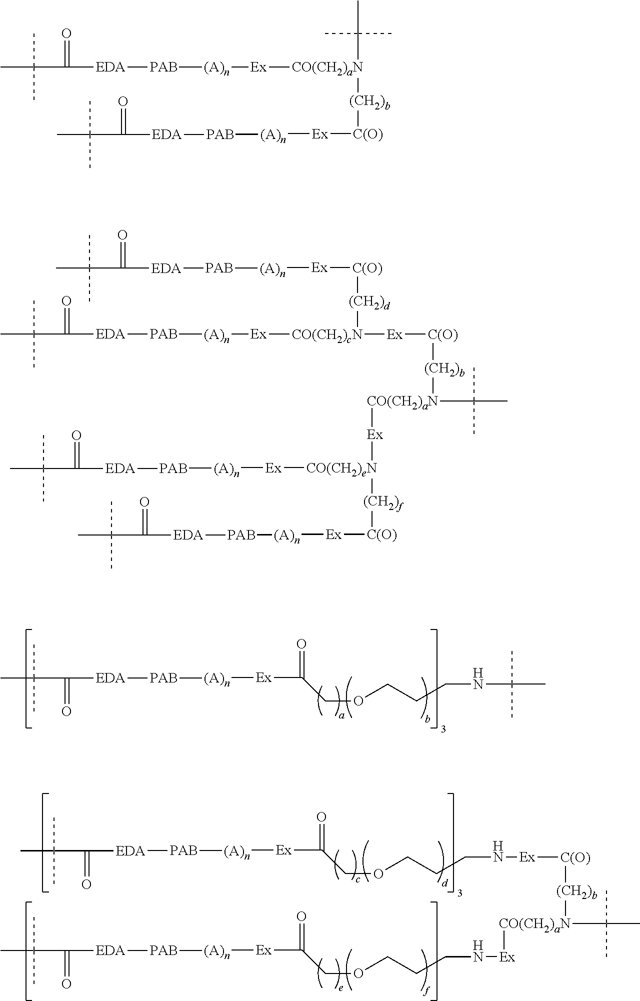
wherein:
each of a, b, c, d, e and f is independently an integer selected from 1-25;
(A)ₙ is a trigger unit of amino acid sequence such as Val-Cit, Val-Ala, Val-Lys, Phe-Lys, Phe-Cit, Phe-Arg, Phe-Ala, Ala-Lys, Leu-Cit, Ile-Cit, Trp-Cit, D-Phe-Phe-Lys, Phe-Phe-Lys, Gly-Phe-Lys, Gly-Phe-Leu-Gly, Gly-Gly-Phe-Gly or Ala-Leu-Ala-Leu;
PAB is para-aminobenzyl alcohol;
EDA is —NR¹(CH₂)ₘNR²—, wherein m is 2 or 3, each of R¹ and R² is independently selected from H, a low molecule weight alkyl or —(CH₂CH₂O)ₗ—CH₃, wherein 1 is an integer selected from 1-10;
each of Ex is an extension spacer comprising a linker chain that is independently selected from:
—NR¹(CH₂)ₓO(CH₂CH₂O)_{y}(CH₂)_{z}C(O)—,
—C(O)(CH₂)ₓNR¹—,
—NR¹(CH₂)ₓO(CH₂CH₂O)_{y}(CH₂)_{z}NR²—,
—NR¹(CH₂)ₓNR²,
—NR¹(CH₂)ₓO(CH₂CH₂O)_{y}(CH₂)_{z}O—,
—O(CH₂)ₓR¹—,
—C(O)(CH₂)ₓO—,
—O(CH₂)ₓO(CH₂CH₂O)_{y}(CH₂)_{z}C(O)—,
—C(O)(CH₂)ₓO(CH₂CH₂O)_{y}(CH₂)_{z}C(O)—,
—C(O)(CH₂)ₓC(O)—,
or Null,
wherein each of x, y, and z is independently an integer selected from 0 to 25; and each of R¹ and R²independently represents hydrogen or a C₁₋₁₀ alkyl group;
alternatively, wherein the branched linker B is selected from

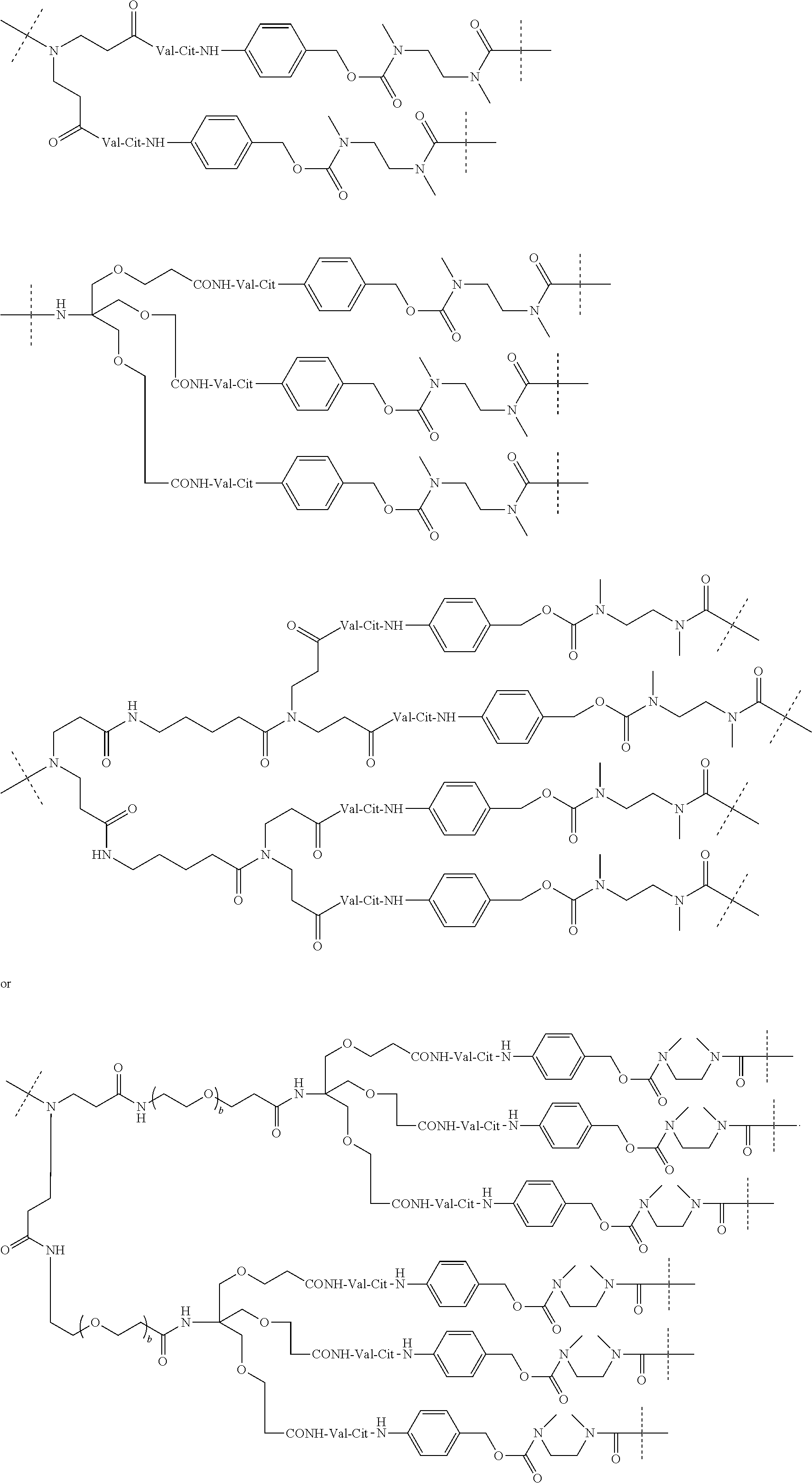

64. The compound of claim 1 selected from the formula: 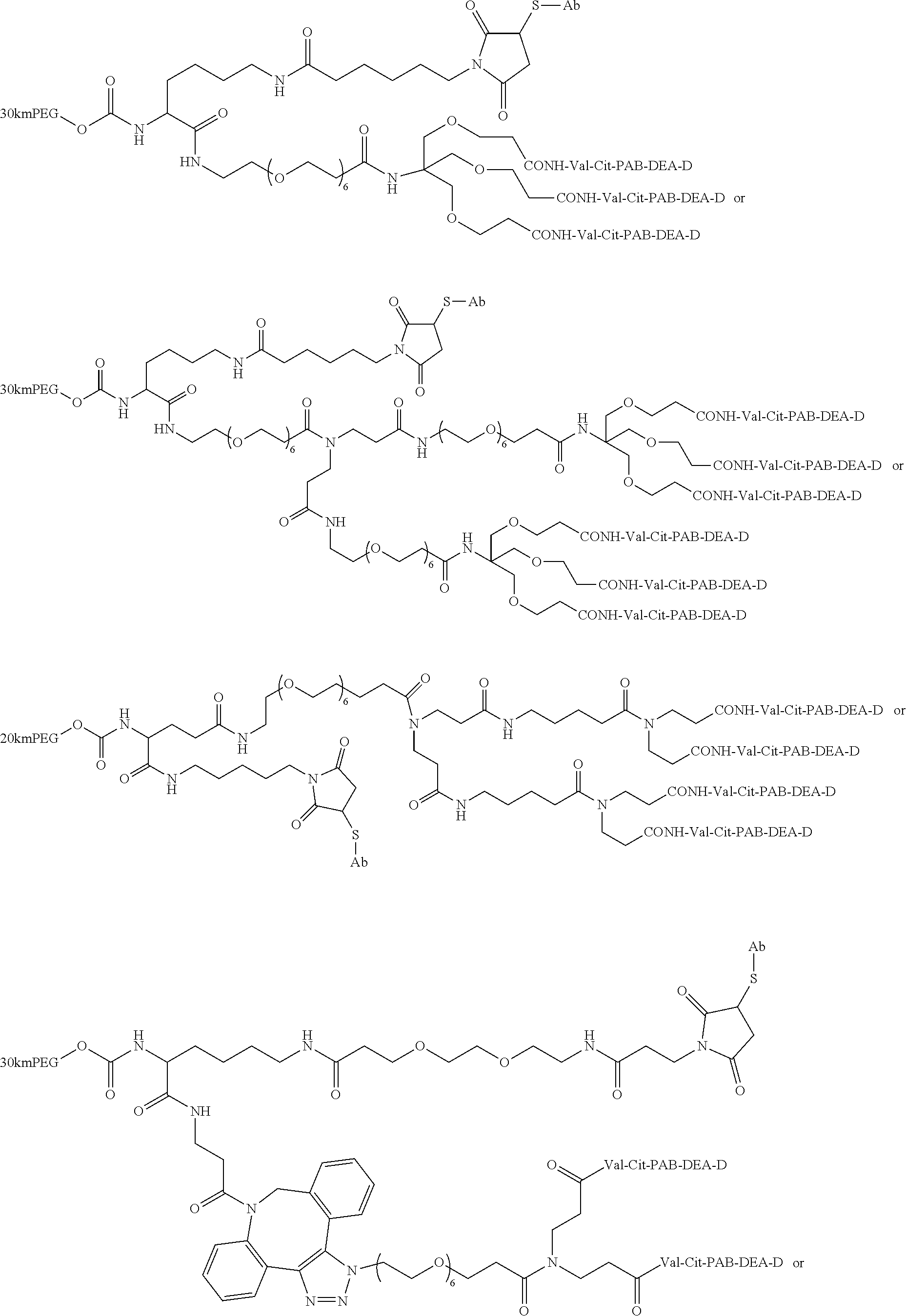 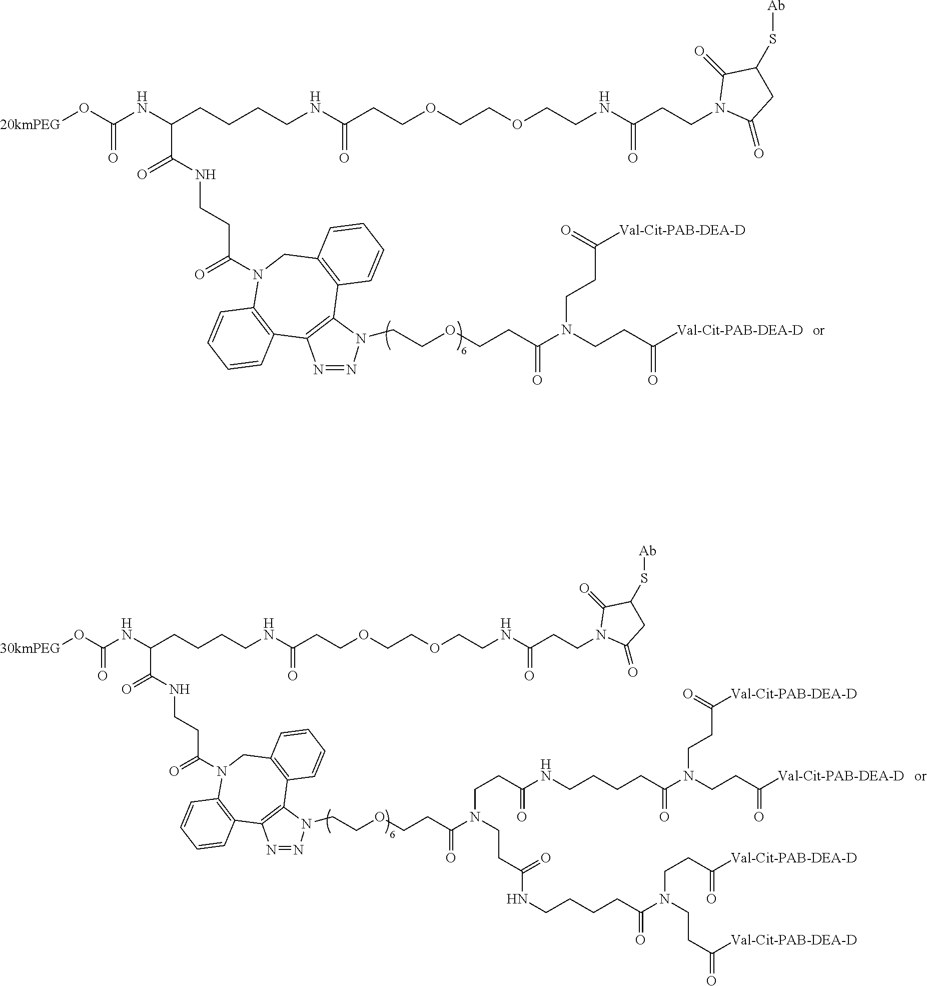 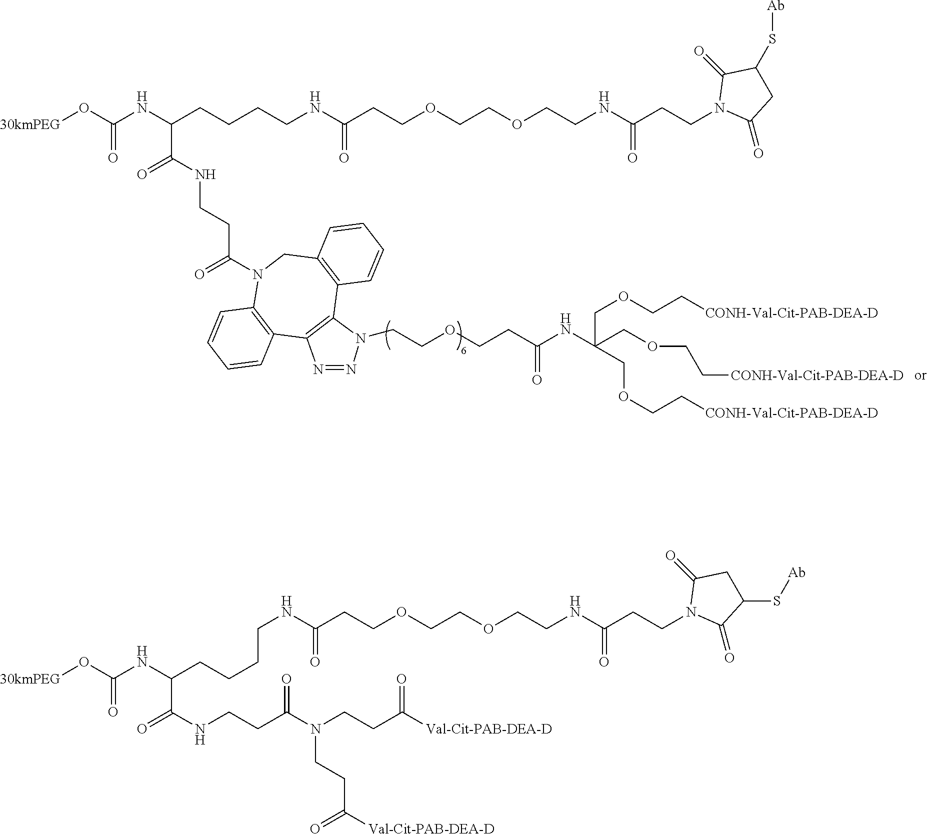
or a pharmaceutically acceptable salt thereof;
wherein Ab is a bispecific antibody targeting PDL1/CD47 or HER2(1)/HER2(2) or cMet(1)/cMet(2) or an antigen binding fragment thereof, 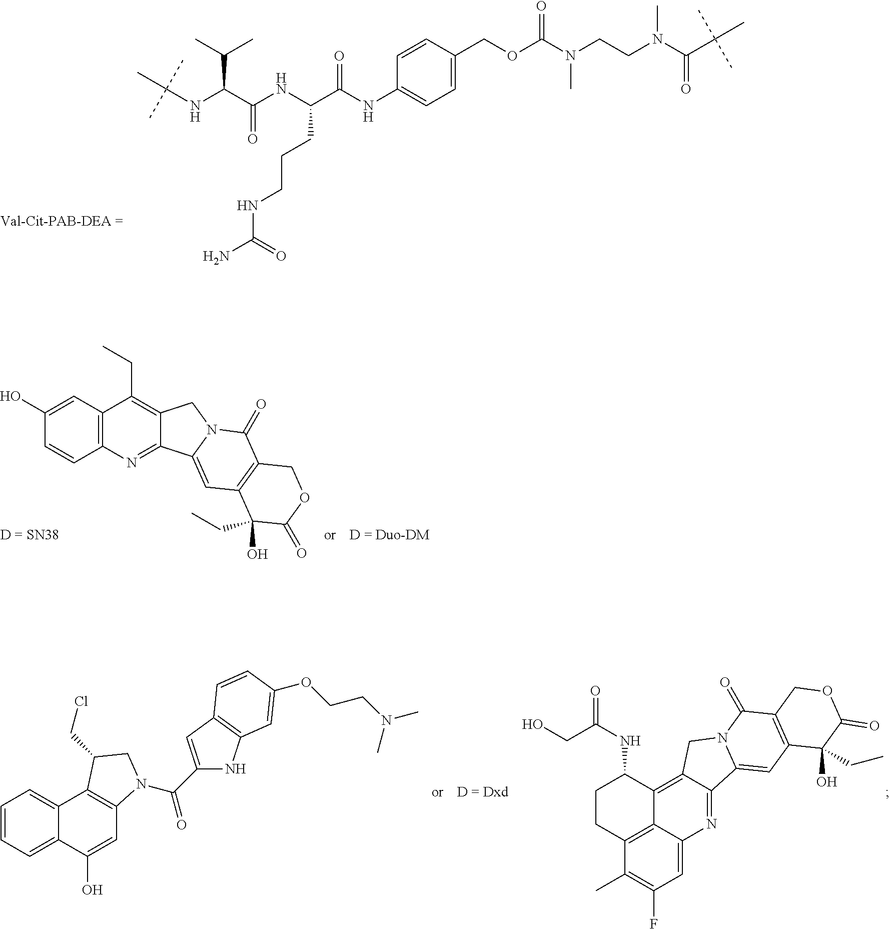
alternatively, wherein the antibody has an amino acid sequence as shown in SEQ ID No. 1 or SEQ ID No. 2 or SEQ ID No. 6.

65. The compound of claim 29 selected from the formula: 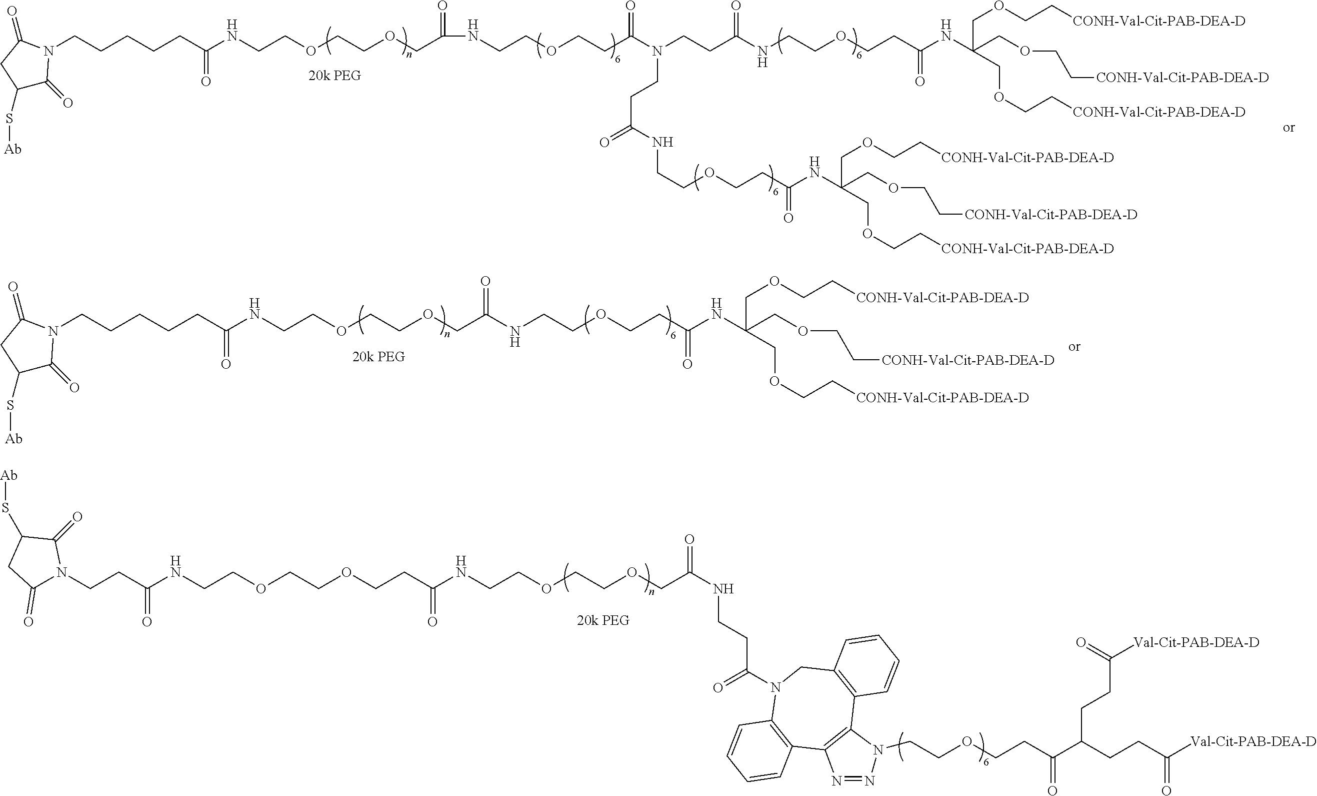
or a pharmaceutically acceptable salt thereof;
wherein Ab is a bispecific antibody targeting PDL1/CD47 or HER2(1)/HER2(2) or cMet(1)/cMet(2) or an antigen binding fragment thereof, 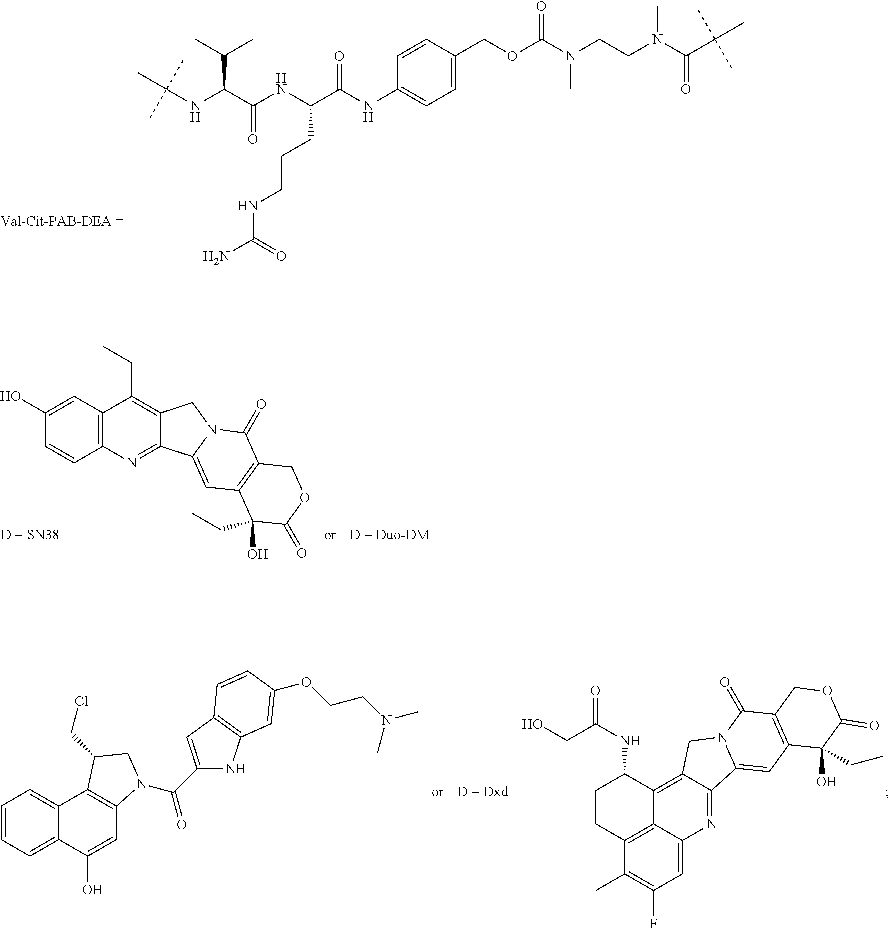
alternatively, wherein the antibody has an amino acid sequence as shown in SEQ ID No. 1 or SEQ ID No. 2 or SEQ ID No. 6.


SEQUENCE LISTING (6 sequences)

SEQ ID NO: 1 - protein, 495 aa, synthetic construct
  source 1..495: note = SCAPDL1xSCACD47
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKGGSGGSGGSGGSGGQVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGCGGSSGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTHHHHHH

SEQ ID NO: 2 - protein, 506 aa, synthetic construct
  source 1..506: note = SCAHer2(1)xSCAHer2(2)
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTGGSGGSGGSGGSGGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGCGSGGSGGSGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGSGGSGGSGGSGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTHHHHHH

SEQ ID NO: 3 - protein, 507 aa, synthetic construct
  source 1..507: note = SCAHer2(1)xSCAHer2(1)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGSGGSGGSGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGCGSGGSGGSGGSGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGSGGSGGSGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSHHHHHH

SEQ ID NO: 4 - protein, 508 aa, synthetic construct
  source 1..508: note = SCAHer2IVxSCAHer3
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGSGGSGGSGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGCGSGGSGGSGGSGGQVQLQQWGAGLLKPSETLSLTCAVYGGSFSGYYWSWIRQPPGKGLEWIGEINHSGSTNTNPSLKSRVTISVETSKNQFSLKLSSVTAADTAVYYCARDKWTWYFDLWGRGTLVTVSSGGSGGSGGSGGSGGDIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKHHHHHH

SEQ ID NO: 5 - protein, 501 aa, synthetic construct
  source 1..501: note = SCAc-Met1xSCAc-Met2
DIQMTQSPSSLSASVGDRVTITCSVSSSVSSIYLHWYQQKPGKAPKLLIYSTSNLASGVPSRFSGSGSGTDFTLTISSLQPEDFATYYCIQYSGYPLTFGGGTKVEIKGGSGGSGGSGGSGGQVQLVQSGAEVKKPGASVKVSCKASGYTFTDYYMHWVRQAPGQGLEWMGRVNPNRGGTTYNQKFEGRVTMTTDTSTSTAYMELRSLRSDDTAVYYCARTNWLDYWGQGTTVTVSGCGSGGSGGSGGSGGQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSGGSGGSGGSGGSGGDIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRHHHHHH

SEQ ID NO: 6 - protein, 519 aa, synthetic construct
  source 1..519: note = SCAc-Met(1)xSCAc-Met(2)
DIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKGGSGGSGGSGGSGGQVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSGCGSGGSGGSGGSGGQVQLVESGPGLVKPSQTLSLTCAVSGGSITTNYYYWSWIRQSPGKGLEWMGVIAYDGSTDYSPSLKSRTSISRDTSKNQFSLQLSSVTPEDTAVYYCARDVRVIATGWATANALDAWGQGTLVTVSSGGSGGSGGSGGSGGQSVLTQPPSVSGSPGKTVTISCAGTSSDVGYGNYVSWYQQLPGTAPKLLIFAVSYRASGIPDRFSGSKSGNTAFLTISGLQSEDEADYYCASYRSSNNAAVFGGGTHLTVLHHHHHH